(19) United States
(12) Patent Application Publication
(10) Pub. No.: US 2022/0195450 A1
(43) Pub. Date: Jun. 23, 2022

(54) METHODS AND COMPOSITIONS FOR GENERATING DOMINANT SHORT STATURE ALLELES USING GENOME EDITING

(71) Applicant: MONSANTO TECHNOLOGY LLC, St. Louis, MO (US)

(72) Inventors: Sivalinganna MANJUNATH, Chesterfield, MO (US); Linda A. RYMARQUIS, High Ridge, MO (US); Thomas L. SLEWINSKI, Chesterfield, MO (US)

(73) Assignee: MONSANTO TECHNOLOGY LLC, St. Louis, MO (US)

(21) Appl. No.: 17/613,114

(22) PCT Filed: May 28, 2020
(86) PCT No.: PCT/US2020/034996

§ 371 Date: Nov. 22, 2021

Related U.S. Application Data
(60) Provisional application No. 62/854,142, filed on May 29, 2019.
(60) Provisional application No. 62/886,732, filed on Aug. 14, 2019.

(51) Int. Cl. C12N 15/82; C12N 9/02

(52) U.S. Cl. CPC C12N 15/8262; C12N 15/8218; C12Y 114/11012; C12N 9/0071; C12N 15/8297

(57) ABSTRACT

The present disclosure provides compositions and methods for altering gibberellin (GA) content in corn or other cereal plants. Methods and compositions are also provided for altering the expression of genes related to gibberellin biosynthesis through editing of a specific GA20 oxidase gene or locus to produce a genomic deletion or disruption that brings an antisense sequence of the GA20 oxidase gene under the control of a neighboring SAMT gene promoter. Modified plant cells and plants having a dominant allele reducing the expression or activity of one or more GA oxidase genes are further provided comprising reduced gibberellin levels and improved characteristics, such as reduced plant height and increased lodging resistance, but without off-types.

### FIELD

This application claims the benefit of U.S. Provisional Application No. 62/854,142, filed May 29, 2019, U.S. Provisional Application No. 62/886,732, filed Aug. 14, 2019, which are incorporated by reference in their entireties herein.

### FIELD

The present disclosure relates to methods and compositions for generating dominant alleles via targeted editing of genomes.

### INCORPORATION OF SEQUENCE LISTING

A sequence listing contained in the file named P34746WO00_SL.txt, which is 120,530 bytes (measured in MS-Windows®) and created on May 28, 2020, and comprises 105 sequences, is filed electronically herewith and incorporated by reference in its entirety.

### BACKGROUND

Gibberellins (gibberellic acids or GAs) are plant hormones that regulate a number of major plant growth and developmental processes. Manipulation of GA levels in semi-dwarf wheat, rice and sorghum plant varieties led to increased yield and reduced lodging in these cereal crops during the 20^{th} century, which was largely responsible for the Green Revolution. However, successful yield gains in other cereal crops, such as corn, through manipulation of the GA pathway, have been challenging. There continues to be a need in the art for the development of monocot or cereal crop plants, such as corn, having increased yield and/or resistance to lodging.

### SUMMARY

In an aspect, the present disclosure provides a modified corn plant, or plant part thereof, comprising a mutant allele of the endogenous GA20 oxidase_5 locus, wherein the mutant allele comprises a genome modification comprising a deletion of at least a portion of the transcription termination sequence of the endogenous Zm.SAMT gene, and wherein the mutant allele produces a RNA molecule comprising an antisense sequence complementary to all or part of the sense strand of the endogenous GA20 oxidase_5 gene.

In an aspect, the present disclosure provides a modified corn plant, or plant part thereof, comprising a mutant allele of the endogenous GA20 oxidase_5 locus, wherein the mutant allele comprises a genome modification comprising a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene.

In an aspect, the present disclosure provides a modified corn plant, or plant part thereof, comprising a mutant allele of the endogenous GA20 oxidase_5 locus, wherein the mutant allele comprises a genome modification comprising a deletion of at least a portion of one or more of the following: 5′ UTR, 1^{st} exon, 1^{st} intron, 2^{nd} exon, 2^{nd} intron, 3^{rd} exon, 3′ UTR, and any portion thereof, and the 5′ UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any portion thereof, of the endogenous Zm.SAMT gene.

In an aspect, the present disclosure provides a modified corn plant, or plant part thereof, comprising a mutant allele of the endogenous GA20 oxidase_5 locus, wherein the mutant allele comprises a genome modification which results in the transcription of an antisense strand of at least an exon, an intron, or an untranslated region (UTR) of the endogenous GA20 oxidase_5 gene, or any portion thereof.

In an aspect, the present disclosure provides a modified corn plant, or plant part thereof, comprising a mutant allele of the endogenous GA20 oxidase_5 locus, wherein the mutant allele comprises the Zm.SAMT gene promoter, or a functional part thereof, operably linked to at least one transcribable antisense sequence of at least an exon, intron or untranslated region (UTR) of the endogenous GA20 oxidase_5 gene, or any portion thereof.

In an aspect, the present disclosure provides a modified corn plant, or plant part thereof, comprising a mutant allele of the endogenous GA20 oxidase_5 locus, wherein the mutant allele comprises a sequence selected from the group consisting of SEQ ID NOs: 87-105.

In an aspect, the present disclosure provides a modified corn plant, or plant part thereof, comprising a mutant allele of the endogenous GA20 oxidase_5 locus, wherein the mutant allele comprises a first sequence and a second sequence; wherein the first sequence comprises one or more of the 5′ UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′ UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any complementary sequence thereof, and any portion of the foregoing, of the endogenous Zm.SAMT gene; wherein the first sequence and the second sequence are contiguous or separated only by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.

In an aspect, the present disclosure provides a modified corn plant, or plant part thereof, comprising a mutant allele of the endogenous GA20 oxidase_5 locus, wherein the mutant allele comprises a genomic deletion relative to a wild type allele of the endogenous GA20 oxidase_5 locus, wherein the genomic deletion is flanked by a first sequence and a second sequence; wherein the first sequence comprises one or more of the 5′ UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′ UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any complementary sequence thereof, and any portion of the foregoing, of the endogenous Zm.SAMT gene.

In an aspect, the present disclosure provides a modified corn plant, or plant part thereof, comprising a mutant allele of the endogenous GA20 oxidase_5 locus, wherein the mutant allele comprises a genomic sequence comprising a first sequence and a second sequence; wherein the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 11-18 and 59-66; wherein the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 18-38 and 39-59; and wherein the genomic sequence is at least 50, at least 75, at least 100, at least 150, at least 200, at least 300, at least 400, at least 500, at least 750, at least 1000, at least 1500, at least 2000, at least 2500, at least 3000, at least 3500, at least 4000, at least 4500, or at least 5000, at least 5500, at least 6000, at least 6500, at least 7000, at least 7500, or at least 8000 consecutive nucleotides in length, and/or fewer than 9000, fewer than 8500, fewer than 8000, fewer than 7500, fewer than 7000, fewer than 6500, fewer than 6000, fewer than 5500, fewer than 5000, fewer than 4500, fewer than 4000, fewer than 3500, fewer than 3000, fewer than 2500, fewer than 2000, fewer than 1500, fewer than 1000, fewer than 750, fewer than 500, fewer than 250, fewer than 200, fewer than 150, fewer than 100, or fewer than 50 consecutive nucleotides in length.

In an aspect, the present disclosure provides a method for producing a modified corn plant comprising a mutant allele of the endogenous GA20 oxidase_5 locus, the method comprising: (a) generating two double-stranded breaks (DSB) in or near the endogenous GA20 oxidase_5 locus in a corn cell using a targeted editing technique; (b) developing or regenerating from the corn cell a corn plant, or plant part thereof, comprising a mutant allele of the endogenous GA20 oxidase_5 locus.

In an aspect, the present disclosure provides a method for producing a modified corn plant comprising a mutant allele of the endogenous GA20 oxidase_5 locus, the method comprising: (a) generating a first and a second double-stranded breaks (DSB) in a corn cell using a targeted editing technique, wherein the first DSB is in a region selected from the group consisting of 5′ UTR, 1^{st} exon, 1^{st} intron, 2^{nd} exon, 2^{nd} intron, 3^{rd} exon, 3′ UTR, and any portion of the foregoing, of the endogenous GA20 oxidase_5 locus, and the intergenic region between the endogenous Zm.GA20 oxidase_5 gene and the endogenous Zm.SAMT gene; wherein the second DSB is in a region selected from the group consisting of 5′ UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any portion of the foregoing, of the endogenous Zm.SAMT locus, and the intergenic region between the endogenous Zm.GA20 oxidase_5 gene and the endogenous Zm.SAMT gene; (a) developing or regenerating from the corn cell a corn plant, or plant part thereof, comprising a genomic deletion, wherein the genomic deletion is flanked by the first DSB and the second DSB.

A method for generating a corn plant comprising: (a) fertilizing at least one female corn plant with pollen from a male corn plant, where the at least one female corn plant and/or the male corn plant comprise(s) a mutant allele of the endogenous GA20 oxidase_5 locus, wherein the mutant allele comprises a genome modification comprising: (i) a deletion of at least a portion of the transcription termination sequence of the endogenous Zm.SAMT gene, and where the mutant allele produces a RNA molecule comprising an antisense sequence complementary to all or part of the sense strand of the endogenous GA20 oxidase_5 gene; (ii) a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene; or (iii) a deletion of at least a portion of one or more of the following: 5′ UTR, 1^{st} exon, 1^{st} intron, 2^{nd} exon, 2^{nd} intron, 3^{rd} exon, 3′ UTR, and any portion thereof, and the 5′ UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any portion thereof, of the endogenous Zm.SAMT gene; and (b) obtaining at least one seed produced by said fertilizing of step (a).

In an aspect, the present disclosure provides a modified corn plant part, corn cell, or corn tissue comprising a mutant allele of the endogenous GA20 oxidase_5 locus, wherein the mutant allele comprises a genome modification comprising a deletion of at least a portion of the transcription termination sequence of the endogenous Zm.SAMT gene, and wherein the mutant allele produces a RNA molecule comprising an antisense sequence complementary to all or part of the sense strand of the endogenous GA20 oxidase_5 gene.

In an aspect, the present disclosure provides a modified corn plant part, corn cell, or corn tissue comprising a mutant allele of the endogenous GA20 oxidase_5 locus, wherein the mutant allele comprises a genome modification comprising a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene.

In an aspect, the present disclosure provides a modified corn plant part, corn cell, or corn tissue comprising a mutant allele of the endogenous GA20 oxidase_5 locus, wherein the mutant allele comprises a genome modification comprising a deletion of at least a portion of one or more of the following: 5′UTR, 1^{st} exon, 1^{st} intron, 2^{nd} exon, 2^{nd} intron, 3^{rd} exon, 3′ UTR, and any portion thereof, and the 5′UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any portion thereof, of the endogenous Zm.SAMT gene.

In an aspect, the present disclosure provides a modified corn plant part, corn cell, or corn tissue comprising a mutant allele of the endogenous GA20 oxidase_5 locus, wherein the mutant allele comprises a genome modification which results in the transcription of an antisense strand of at least an exon, an intron, or an untranslated region (UTR) of the endogenous GA20 oxidase_5 gene, or any portion thereof.

In an aspect, the present disclosure provides a modified corn plant part, corn cell, or corn tissue comprising a mutant allele of the endogenous GA20 oxidase_5 locus, wherein the mutant allele comprises the Zm.SAMT gene promoter, or a functional part thereof, operably linked to at least one transcribable antisense sequence of at least an exon, intron or untranslated region (UTR) of the endogenous GA20 oxidase_5 gene, or any portion thereof.

In an aspect, the present disclosure provides a modified corn plant part, corn cell, or corn tissue comprising a mutant allele of the endogenous GA20 oxidase_5 locus, wherein the mutant allele comprises a sequence selected from the group consisting of SEQ ID NOs: 87-105.

In an aspect, the present disclosure provides a modified corn plant part, corn cell, or corn tissue comprising a mutant allele of the endogenous GA20 oxidase_5 locus, wherein the mutant allele comprises a first sequence and a second sequence; wherein the first sequence comprises one or more of the 5′UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any complementary sequence thereof, and any portion of the foregoing, of the endogenous Zm.SAMT gene; wherein the first sequence and the second sequence are contiguous or separated only by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.

In an aspect, the present disclosure provides a modified corn plant part, corn cell, or corn tissue comprising a mutant allele of the endogenous GA20 oxidase_5 locus, wherein the mutant allele comprises a genomic deletion relative to a wild type allele of the endogenous GA20 oxidase_5 locus, wherein the genomic deletion is flanked by a first sequence and a second sequence; wherein the first sequence comprises one or more of the 5′UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′UTR, 1^{st} exon, 1^{st} intron, 2^{nd} exon, 2^{nd} intron, 3^{rd} exon, 3^{rd} intron, 4th exon, 4th intron, 5th exon, 5th intron, 6th exon, 6th intron, 7th exon, 7th intron, 8th exon, 3′ UTR, and any complementary sequence thereof, and any portion of the foregoing, of the endogenous Zm.SAMT gene.

In an aspect, the present disclosure provides a modified corn plant part, corn cell, or corn tissue comprising a mutant allele of the endogenous GA20 oxidase_5 locus, wherein the mutant allele comprises a genomic sequence comprising a first sequence and a second sequence; wherein the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 11-18 and 59-66; wherein the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 18-38 and 39-59; and wherein the genomic sequence is at least 50, at least 75, at least 100, at least 150, at least 200, at least 300, at least 400, at least 500, at least 750, at least 1000, at least 1500, at least 2000, at least 2500, at least 3000, at least 3500, at least 4000, at least 4500, or at least 5000, at least 5500, at least 6000, at least 6500, at least 7000, at least 7500, or at least 8000 consecutive nucleotides in length, and/or fewer than 9000, fewer than 8500, fewer than 8000, fewer than 7500, fewer than 7000, fewer than 6500, fewer than 6000, fewer than 5500, fewer than 5000, fewer than 4500, fewer than 4000, fewer than 3500, fewer than 3000, fewer than 2500, fewer than 2000, fewer than 1500, fewer than 1000, fewer than 750, fewer than 500, fewer than 250, fewer than 200, fewer than 150, fewer than 100, or fewer than 50 consecutive nucleotides in length.

### BRIEF DESCRIPTION OF THE DRAWINGS

FIG. 1 provides illustrative examples for creating an antisense RNA molecule that targets the Zm.GA20ox5 gene and the Zm.GA20ox3 gene by deleting a genomic region between the Zm.GA20ox5 and its neighboring gene Zm.SAMT oriented in the opposite direction, through genome editing.

FIG. 2 illustrates the genomic position of various guide RNA target sites in three exemplified vectors for creating a genomic deletion between the Zm.GA20ox5 gene and its neighboring Zm.SAMT gene.

FIG. 3 depicts the average height of wild type plants and homozygous edited plants in inches (Y-axis).

FIG. 4 depicts the average height of wild type plants and homozygous or heterozygous edited plants in inches (Y-axis).

FIG. 5 depicts the concentration of GA12 and GA9 in pmol/g (Y-axis) in edited and control plants.

FIG. 6 depicts the concentration of GA20 and GA53 in pmol/g (Y-axis) in edited and control plants.

FIG. 7 depicts the concentration of the active gibberellic acids GA1, GA3, and GA4 in pmol/g (Y-axis) in edited and control plants.

### DETAILED DESCRIPTION

Unless defined otherwise herein, terms are to be understood according to their conventional usage by those of ordinary skill in the relevant art. To facilitate understanding of the disclosure, several terms and abbreviations as used herein are defined below as follows:

The term “and/or” when used in a list of two or more items, means that any one of the listed items can be employed by itself or in combination with any one or more of the listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

The term “about” as used herein, is intended to qualify the numerical values that it modifies, denoting such a value as variable within a margin of error. When no particular margin of error, such as a standard deviation to a mean value, is recited, the term “about” should be understood to mean that range which would encompass the recited value and the range which would be included by rounding up or down to that figure, taking into account significant figures.

As used herein, a “plant” includes an explant, plant part, seedling, plantlet or whole plant at any stage of regeneration or development. The term “cereal plant” as used herein refers a monocotyledonous (monocot) crop plant that is in the Poaceae or Gramineae family of grasses and is typically harvested for its seed, including, for example, wheat, corn, rice, millet, barley, sorghum, oat and rye. As commonly understood, a “corn plant” or “maize plant” refers to any plant of species Zea mays and includes all plant varieties that can be bred with corn, including wild maize species.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

As used herein, a “locus” is a chromosomal locus or region where a polymorphic nucleic acid, trait determinant, gene, or marker is located. A “locus” can be shared by two homologous chromosomes to refer to their corresponding locus or region.

As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have a reduced or eliminated activity or expression level for the gene relative to the wild-type allele. According to present embodiments, a mutant or edited allele for a GA20 oxidase 5 gene may have a deletion between the endogenous GA20 oxidase 5 and SAMT genes. For diploid organisms such as corn, a first allele can occur on one chromosome, and a second allele can occur at the same locus on a second homologous chromosome. If one allele at a locus on one chromosome of a plant is a mutant or edited allele and the other corresponding allele on the homologous chromosome of the plant is wild-type, then the plant is described as being heterozygous for the mutant or edited allele. However, if both alleles at a locus are mutant or edited alleles, then the plant is described as being homozygous for the mutant or edited alleles. A plant homozygous for mutant or edited alleles at a locus may comprise the same mutant or edited allele or different mutant or edited alleles if heteroallelic or biallelic.

As used herein, an “endogenous locus” refers to a locus at its natural and original chromosomal location. As used herein, the “endogenous GA20 oxidase_3 locus” refers to the GA20 oxidase_3 genic locus at its original chromosomal location. As used herein, the “endogenous GA20 oxidase_5 locus” refers to the GA20 oxidase_5 genic locus at its original chromosomal location.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, in the context of a protein-coding gene, an “exon” refers to a segment of a DNA or RNA molecule containing information coding for a protein or polypeptide sequence.

As used herein, an “intron” of a gene refers to a segment of a DNA or RNA molecule, which does not contain information coding for a protein or polypeptide, and which is first transcribed into a RNA sequence but then spliced out from a mature RNA molecule.

As used herein, an “untranslated region (UTR)” of a gene refers to a segment of a RNA molecule or sequence (e.g., a mRNA molecule) expressed from a gene (or transgene), but excluding the exon and intron sequences of the RNA molecule. An “untranslated region (UTR)” also refers a DNA segment or sequence encoding such a UTR segment of a RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, the term “expression” refers to the biosynthesis of a gene product, and typically the transcription and/or translation of a nucleotide sequence, such as an endogenous gene, a heterologous gene, a transgene or a RNA and/or protein coding sequence, in a cell, tissue, organ, or organism, such as a plant, plant part or plant cell, tissue or organ.

As used herein, a “transcription termination sequence” refers to a nucleic acid sequence containing a signal that triggers the release of a newly synthesized transcript RNA molecule from a RNA polymerase complex and marks the end of transcription of a gene or locus.

As used herein, a “wild-type gene” or “wild-type allele” refers to a gene or allele having a sequence or genotype that is most common in a particular plant species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. For purposes of calculating “percent identity” between DNA and RNA sequences, a uracil (U) of a RNA sequence is considered identical to a thymine (T) of a DNA sequence. If the window of comparison is defined as a region of alignment between two or more sequences (i.e., excluding nucleotides at the 5′ and 3′ ends of aligned polynucleotide sequences, or amino acids at the N-terminus and C-terminus of aligned protein sequences, that are not identical between the compared sequences), then the “percent identity” may also be referred to as a “percent alignment identity”. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

For optimal alignment of sequences to calculate their percent identity, various pair-wise or multiple sequence alignment algorithms and programs are known in the art, such as ClustalW, or Basic Local Alignment Search Tool® (BLAST®), etc., that may be used to compare the sequence identity or similarity between two or more nucleotide or protein sequences. Although other alignment and comparison methods are known in the art, the alignment between two sequences (including the percent identity ranges described above) may be as determined by the ClustalW or BLAST® algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); and Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and a RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, with respective to a given sequence, a “complement”, a “complementary sequence” and a “reverse complement” are used interchangeably. All three terms refer to the inversely complementary sequence of a nucleotide sequence, i.e. to a sequence complementary to a given sequence in reverse order of the nucleotides. As an example, the reverse complement of a nucleotide sequence having the sequence 5′-atggttc-3′ is 5′-gaaccat-3′.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as a RNA molecule transcribed from the gene or locus (with the exception of Uracil in RNA and Thymine in DNA).

As used herein, unless specified otherwise, the relative location of two sequence elements of a genic locus, when expressed as “upstream,” “downstream,” “at the 5′ end,” or “at the 3′ end,” is determined based on the direction of the transcription activity associated with that genic locus. For example, for two transcribed genomic DNA elements, their relative location is based on their sense strand where the first genomic DNA element is upstream or at the 5′ end of the second genomic DNA element when the first genomic DNA element is transcribed first.

The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s). Two transcribable DNA sequences can also be “operably linked” to each other if their transcription is subject to the control of a common promoter or other regulatory element.

As used herein, an “encoding region” or “coding region” refers to a portion of a polynucleotide that encodes a functional unit or molecule (e.g., without being limiting, a mRNA, protein, or non-coding RNA sequence or molecule). An “encoding region” or “coding region” can contain, for example, one or more exons, one or more introns, a 5′-UTR, a 3′-UTR, or any combination thereof.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE (transcription activator-like effector)-endonuclease (TALEN), a recombinase, or a transposase. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” also encompasses the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution or insertion, whereas “edits” or “genomic edits” refers to two or more targeted mutation(s), deletion(s), inversion(s), substitution(s) and/or insertion(s), with each “edit” being introduced via a targeted genome editing technique.

As used herein, “modified” in the context of a plant, plant seed, plant part, plant cell, and/or plant genome, refers to a plant, plant seed, plant part, plant cell, and/or plant genome comprising an engineered change in the expression level and/or coding sequence of one or more genes of interest relative to a wild-type or control plant, plant seed, plant part, plant cell, and/or plant genome. Indeed, the term “modified” may further refer to a plant, plant seed, plant part, plant cell, and/or plant genome having one or more deletions affecting expression of one or more endogenous GA oxidase genes, such as one or more endogenous GA20 oxidase genes, introduced through chemical mutagenesis, transposon insertion or excision, or any other known mutagenesis technique, or introduced through genome editing. In an aspect, a modified plant, plant seed, plant part, plant cell, and/or plant genome can comprise one or more transgenes. For clarity, therefore, a modified plant, plant seed, plant part, plant cell, and/or plant genome includes a mutated, edited and/or transgenic plant, plant seed, plant part, plant cell, and/or plant genome having a modified expression level, expression pattern, and/or coding sequence of one or more GA oxidase gene(s) relative to a wild-type or control plant, plant seed, plant part, plant cell, and/or plant genome. Modified plants can be homozygous or heterozygous for any given mutation or edit, and/or may be bi-allelic or heteroallelic at a GA oxidase gene locus. A modified plant is bi-allelic or heteroallelic for a GA oxidase gene if each copy of the GA oxidase gene is a different allele (i.e., comprises different mutation(s) and/or edit(s)), wherein each allele lowers the expression level and/or activity of the GA oxidase gene. Modified plants, plant parts, seeds, etc., may have been subjected to mutagenesis, genome editing or site-directed integration (e.g., without being limiting, via methods using site-specific nucleases), genetic transformation (e.g., without being limiting, via methods of Agrobacterium transformation or microprojectile bombardment), or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the one or more GA oxidase genes. A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

As used herein, the term “control plant” (or likewise a “control” plant seed, plant part, plant cell and/or plant genome) refers to a plant (or plant seed, plant part, plant cell and/or plant genome) that is used for comparison to a modified plant (or modified plant seed, plant part, plant cell and/or plant genome) and has the same or similar genetic background (e.g., same parental lines, hybrid cross, inbred line, testers, etc.) as the modified plant (or plant seed, plant part, plant cell and/or plant genome), except for genome edit(s) (e.g., a deletion) affecting one or more GA oxidase genes. For example, a control plant may be an inbred line that is the same as the inbred line used to make the modified plant, or a control plant may be the product of the same hybrid cross of inbred parental lines as the modified plant, except for the absence in the control plant of any transgenic events or genome edit(s) affecting one or more GA oxidase genes. Similarly, an unmodified control plant refers to a plant that shares a substantially similar or essentially identical genetic background as a modified plant, but without the one or more engineered changes to the genome (e.g., transgene, mutation or edit) of the modified plant. For purposes of comparison to a modified plant, plant seed, plant part, plant cell and/or plant genome, a “wild-type plant” (or likewise a “wild-type” plant seed, plant part, plant cell and/or plant genome) refers to a non-transgenic and non-genome edited control plant, plant seed, plant part, plant cell and/or plant genome. As used herein, a “control” plant, plant seed, plant part, plant cell and/or plant genome may also be a plant, plant seed, plant part, plant cell and/or plant genome having a similar (but not the same or identical) genetic background to a modified plant, plant seed, plant part, plant cell and/or plant genome, if deemed sufficiently similar for comparison of the characteristics or traits to be analyzed.

As used herein, a “target site” for genome editing refers to the location of a polynucleotide sequence within a plant genome that is bound and cleaved by a site-specific nuclease introducing a double stranded break (or single-stranded nick) into the nucleic acid backbone of the polynucleotide sequence and/or its complementary DNA strand. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for a RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further below). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by another site-specific nuclease that may not be guided by a non-coding RNA molecule, such as a meganuclease, zinc finger nuclease (ZFN), or a transcription activator-like effector nuclease (TALEN), to introduce a double stranded break (or single-stranded nick) into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region.

As used herein, the terms “suppress,” “suppression,” “inhibit,” “inhibition,” “inhibiting”, and “downregulation” refer to a lowering, reduction or elimination of the expression level of a mRNA and/or protein encoded by a target gene in a plant, plant cell or plant tissue at one or more stage(s) of plant development, as compared to the expression level of such target mRNA and/or protein in a wild-type or control plant, cell or tissue at the same stage(s) of plant development. A target gene may be suppressed in a plant or plant tissue through one or more different mechanisms as provided herein. According to some embodiments, a modified plant is provided having a GA20 oxidase gene expression level, such as a GA20 oxidase 5 and/or GA20 oxidase 3 gene expression level(s), that is/are reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a GA20 oxidase gene expression level, such as a GA20 oxidase 5 and/or GA20 oxidase 3 gene expression level(s), that is/are reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

According to some embodiments, a modified plant is provided having a GA20 oxidase mRNA level, such as a GA20 oxidase 5 and/or GA20 oxidase 3 mRNA level(s), that is/are reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a GA20 oxidase mRNA expression level, such as a GA20 oxidase 5 and/or GA20 oxidase 3 mRNA level(s), that is/are reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a modified plant is provided having a GA20 oxidase protein expression level, such as a GA20 oxidase 5 and/or GA20 oxidase 3 protein level(s), that is/are reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a GA20 oxidase protein expression level, such as a GA20 oxidase 5 and/or GA20 oxidase 3 protein level(s), that is/are reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

As used herein, an “intergenic region” or “intergenic sequence” refers to a genomic region or a polynucleotide sequence located in between transcribed regions of two neighboring genes. For example, the endogenous Zm.GA20ox5 gene and its neighboring gene in the corn or maize genome, the s-adenosyl methyl transferase (SAMT) or Zm.SAMT gene, contains an intergenic region between the 3′ UTR of the Zm.GA20ox5 gene and the 3′ UTR of the Zm.SAMT gene.

Recently, the suppression of the GA20 oxidase_3 and GA20 oxidase_5 genes via an artificial microRNA or gene editing was reported in corn. See co-pending PCT Application No. PCT/US2017/047405 and U.S. application Ser. No. 15/679,699, both filed on Aug. 17, 2017, and co-pending PCT Application Nos. PCT/US2019/018128, PCT/US2019/018131, and PCT/US2019/018133, all filed on Feb. 15, 2019, all incorporated herein by reference in their entirety.

GA oxidases in cereal plants consist of a family of related GA oxidase genes. For example, corn has a family of at least nine GA20 oxidase genes that includes GA20 oxidase_1, GA20 oxidase_2, GA20 oxidase_3, GA20 oxidase_4, GA20 oxidase_5, GA20 oxidase_6, GA20 oxidase_7, GA20 oxidase_8, and GA20 oxidase_9. The DNA and protein sequences by SEQ ID NOs for each of GA20 oxidase_3 and GA20 oxidase_5 are provided in Table 1.

TABLE 1
DNA and protein sequences by sequence identifier for GA20
oxidase_3 and GA20 oxidase_5 genes in corn.

| GA20 oxidase | Genomic | | Coding | |
|---|---|---|---|---|
| Gene | DNA | cDNA | Sequence (CDS) | Protein |
| GA20 oxidase_3 | SEQ ID NO: 1 | SEQ ID NO: 2 | SEQ ID NO: 3 | SEQ ID NO: 4 |
| GA20 oxidase_5 | SEQ ID NO: 5 | SEQ ID NO: 6 | SEQ ID NO: 7 | SEQ ID NO: 8 |

A wild-type genomic DNA sequence of the GA20 oxidase_3 locus from a reference genome is provided in SEQ ID NO: 1, and A wild-type genomic DNA sequence of the GA20 oxidase_5 locus from a reference genome is provided in SEQ ID NO: 5.

For the corn GA20 oxidase_3 gene (also referred to as Zm.GA20ox3), SEQ ID NO: 1 provides 3000 nucleotides upstream (5′) of the GA20 oxidase_3 5′-UTR; nucleotides 3001-3096 correspond to the 5′-UTR; nucleotides 3097-3665 correspond to the first exon; nucleotides 3666-3775 correspond to the first intron; nucleotides 3776-4097 correspond to the second exon; nucleotides 4098-5314 correspond to the second intron; nucleotides 5315-5584 correspond to the third exon; and nucleotides 5585-5800 correspond to the 3′-UTR. SEQ ID NO: 1 also provides 3000 nucleotides downstream (3′) of the end of the 3′-UTR (nucleotides 5801-8800).

For the corn GA20 oxidase_5 gene (also referred to as Zm.GA20ox5), SEQ ID NO: 5 provides 3000 nucleotides upstream of the GA20 oxidase_5 start codon (nucleotides 1-3000); nucleotides 3001-3791 correspond to the first exon; nucleotides 3792-3906 correspond to the first intron; nucleotides 3907-4475 correspond to the second exon; nucleotides 4476-5197 correspond to the second intron; nucleotides 5198-5473 correspond to the third exon; and nucleotides 5474-5859 correspond to the 3′-UTR. SEQ ID NO: 5 also provides 3000 nucleotides downstream (3′) of the end of the 3′-UTR (nucleotides 5860-8859).

In the corn genome, the Zm.GA20ox5 gene located next to the Zm.SAMT gene. These two genes are separated by an intergenic region of about 550 bp, with the Zm.SAMT gene positioned downstream and oriented in the opposite orientation relative to the Zm.GA20ox5 gene. A reference genomic sequence of the region encompassing the Zm.GA20ox5 and Zm.SAMT genes is provided in SEQ ID NOs. 9 and 10. SEQ ID NO. 9 represents the sequence of the sense strand of the Zm.GA20ox5 gene encompassing both Zm.GA20ox5 and Zm.SAMT genes (the “GA20ox5_SAMT genomic sequence” in Table 2). SEQ ID NO: 9 partially overlaps with SEQ ID NO: 5 and has a shorter Zm.GA20ox5 upstream sequence and a longer Zm.GA20ox5 downstream sequence compared to the SEQ ID NO: 5. SEQ ID NO. 10 represents the sequence of the sense strand of the Zm.SAMT gene (i.e., the antisense strand of the Zm.GA20ox5 gene) encompassing both Zm.GA20ox5 and Zm.SAMT genes (the “SAMT_GA20ox5 genomic sequence” in Table 2). The elements or regions of the reference genomic Zm.GA20ox5/Zm.SAMT sequence are annotated in Table 2 below by reference to the nucleotide coordinates of those elements or regions in SEQ ID NO. 9 or 10.

It was previously shown that suppression of GA20 oxidase gene(s) and/or targeting of a subset of one or more GA oxidase genes via transgenic suppression (e.g., an artificial microRNA-mediated suppression of both GA20 oxidase_3 and GA20 oxidase_5 genes) can be effective in achieving a short stature, semi-dwarf phenotype with increased resistance to lodging, but without reproductive off-types in the ear. See PCT Application No. PCT/US2017/047405 and U.S. application Ser. No. 15/679,699, both filed on Aug. 17, 2017, and published as WO/2018/035354 and US20180051295, respectively. Furthermore, knocking out GA20 oxidase_3, GA20 oxidase_5, or both genes via genome editing also can cause reduced plant height and increased lodging resistance, and impacts GA hormonal levels. See PCT Application Nos. PCT/US2019/018128, PCT/US2019/018131, and PCT/US2019/018133, all filed on Feb. 15, 2019.

Dominant negative alleles are alleles that mask the contribution of a second allele (e.g., a wild-type allele) at the same locus (e.g., a second allele of the same gene) or gene. A dominant allele may be referred to as semi-dominant if the masking effect is partial or incomplete. Sometimes, a dominant allele of one locus or gene can also have dominant effects over another locus or gene. Dominant negative alleles, or antimorphs, of a gene are alleles that produce altered gene products (relative to the wild-type allele of the gene) acting in opposition to wild-type allelic function. For example, a dominant negative allele can abrogate or suppress the normal function of a wild-type allele or gene product in a heterozygous state.

Creation of dominant alleles that work in a heterozygous state, can speed up effective trait development, deployment, and launch of gene editing-derived products in hybrid crops such as corn. Dominant negative alleles have the potential advantage of providing a positive or beneficial plant trait in a heterozygous state—e.g., when present in a single copy. As a result, a dominant negative mutant allele can be introduced through crossing into a progeny plant from a single parent without having to introduce the allele from both parent plants as with a recessive allele. The present disclosure provides methods and compositions to selectively edit a genome of a corn plant to create a dominant negative allele of a GA20ox5 locus or gene that produces a beneficial trait in a plant.

Without being bound by any scientific theory, if a genomic region between the neighboring Zm.GA20ox5 and Zm.SAMT genes (including possibly all or part of those genes) is deleted, then the endogenous Zm.SAMT gene promoter can drive expression of an antisense RNA transcript through all or part of the Zm.GA20ox5 gene that can hybridize to a separate RNA transcript expressed from one or both of the copies or alleles of the Zm.GA20ox5 and/or Zm.GA20ox3 gene(s). Thus, a mutant allele having a deletion between the Zm.GA20ox5 and Zm.SAMT genes can behave as a dominant negative mutation or allele by causing suppression or silencing of one or both (wild-type and/or mutant) copies or alleles of the endogenous Zm.GA20ox5 gene, in addition to possible further suppression or silencing of one or both copies or alleles of the endogenous Zm.GA20ox3 gene.

In an aspect, this disclosure provides a modified corn plant or a method for producing such modified corn plant, where the modified corn plant has a dominant allele (for example, a semi-dominant allele) at the endogenous GA20 oxidase_5 locus or gene, where such dominant allele produces an antisense RNA molecule which suppresses or opposes the expression or function of one or more wide-type alleles of the endogenous GA20 oxidase_3 locus or gene, the endogenous GA20 oxidase_5 locus or gene, or both. In another aspect, an GA20 oxidase_5 dominant allele or dominant negative allele comprises a genome deletion.

Further provided herein are methods of generating dominant alleles or dominant negative alleles of genes or gene regions using targeted genome editing techniques. Also provided herein are cells, tissues, or explants generated by such methods and compositions used in such methods. The instant description further provides modified plants regenerated from cells, tissues, or explants subjected to the methods provided herein, any of their progeny, and any plant parts thereof. In one aspect, a dominant allele or dominant negative allele of a gene provided herein is able to suppress the expression of a wild-type and/or mutant allele(s) of the same and/or different locus/loci or gene(s) in a heterozygous state.

According to aspects of the present disclosure, a mutant or edited allele of the endogenous GA20 oxidase_5 (GA20ox5) gene or locus is provided comprising a deletion between the neighboring Zm.GA20ox5 and Zm.SAMT genes, such that an antisense RNA molecule that is complementary to all or part of the coding sequence of the GA20ox5 gene may be transcribed under the control of the endogenous Zm.SAMT gene promoter. It is contemplated that the antisense RNA molecule transcribed from the mutant or edited allele of the endogenous GA20 oxidase_5 gene or locus may affect the expression level(s) of the GA20 oxidase_5 and/or endogenous GA20 oxidase_3 gene(s) through different mechanisms, such as nonsense mediated decay, non-stop decay, no-go decay, DNA or histone methylation or other epigenetic changes, inhibition or decreased efficiency of transcription and/or translation, ribosomal interference, interference with mRNA processing or splicing, and/or ubiquitin-mediated protein degradation via the proteasome. See, e.g., Nickless, A. et al., “Control of gene expression through the nonsense-mediated RNA decay pathway”, Cell Biosci 7:26 (2017); Karamyshev, A. et al., “Lost in Translation: Ribosome-Associated mRNA and Protein Quality Controls”, Frontiers in Genetics 9:431 (2018); Inada, T., “Quality controls induced by aberrant translation”, Nucleic Acids Res 48:3 (2020); and Szadeczky-Kardoss, I. et al., “The nonstop decay and the RNA silencing systems operate cooperatively in plants”, Nucleic Acids Res 46:9 (2018), the entire contents and disclosures of which are incorporated herein by reference. Each of these different mechanisms may act alternatively or in addition to RNA interference (RNAi), transcriptional gene silencing (PGS) and/or post transcriptional gene silencing (PTGS) mechanisms. See, e.g., Wilson, R. C. et al., “Molecular Mechanisms of RNA Interference”, Annu Rev Biophysics 42:217-39 (2013); and Guo, Q. et al., “RNA Silencing in Plants: Mechanism, Technologies and Applications in Horticulture Crops”, Current Genomics 17:476-489 (2016), the entire contents and disclosures of which is incorporated herein by reference. Some of the above mechanisms may reduce expression of the edited allele itself, while others may also reduce the expression of other copy/-ies or allele(s) of the endogenous GA20 oxidase_5 and/or GA20 oxidase_3 locus/loci or gene(s). Indeed, it is envisioned that the edited endogenous GA20 oxidase_5 locus, gene or allele may not only reduce or eliminate its own expression and/or activity level, but may also have a dominant or semi-dominant effect(s) on the other copy/-ies or allele(s) of the endogenous GA20 oxidase_5 and/or GA20 oxidase_3 locus/loci or gene(s). Such dominant or semi-dominant effect(s) on the GA20 oxidase_5 and/or GA20 oxidase_3 gene(s) may operate through non-canonical suppression mechanisms that do not involve RNAi and/or formation of targeted small RNAs at a significant or detectable level.

In an aspect, the present disclosure provides a modified corn plant, or plant part thereof, comprising a mutant allele of the endogenous GA20 oxidase_5 locus or gene, where the mutant allele comprises a genome modification deleting or disrupting at least a portion of the transcription termination sequence of the endogenous Zm.SAMT locus or gene. In another aspect, a genome modification further deletes or disrupts at least a portion of the transcription termination sequence of the endogenous GA20 oxidase_5 locus or gene. In a further aspect, a genome modification comprises a deletion or disruption of one or both of the transcription termination sequences of the endogenous GA20 oxidase_5 and SAMT genes. In another aspect, a GA20 oxidase_5 mutant allele produces a RNA molecule comprising an antisense sequence that is complementary to at least a portion of a RNA transcript, such as a wild-type RNA transcript, of the endogenous GA20 oxidase_5 locus or gene, and is able to suppress the expression of a wild-type allele of the endogenous GA20 oxidase_5 locus or gene, a wild-type allele of the endogenous GA20 oxidase_3 locus or gene, or both.

In an aspect, the present disclosure provides a modified corn plant, or plant part thereof, comprising a mutant allele of the endogenous GA20 oxidase_5 locus or gene, where the mutant allele comprises a genome modification deleting at least a portion of the transcription termination sequence of the endogenous Zm.SAMT locus or gene, and where the mutant allele produces a RNA molecule comprising an antisense sequence complementary to all or part of the endogenous GA20 oxidase_5 gene. In another aspect, a GA20 oxidase_5 mutant allele comprises the endogenous Zm.SAMT gene promoter, or a functional portion thereof, operably linked to a transcribable DNA sequence encoding a RNA molecule that causes suppression of one or both of the endogenous GA20 oxidase_3 gene and the endogenous GA20 oxidase_5 gene. In a further aspect, a GA20 oxidase_5 mutant allele comprises the endogenous Zm.SAMT gene promoter, or a portion thereof, operably linked to a transcribable DNA sequence encoding a RNA molecule comprising an antisense sequence that is at least 80%, at least 85%, at least 90%, at least 95%, at least 96%, at least 97%, at least 98%, at least 99%, or 100% complementary to all or part of the endogenous GA20 oxidase_3 and/or GA20 oxidase_5 gene(s).

In an aspect, a GA20 oxidase_5 mutant allele comprises a transcribable DNA sequence that is at least 80%, at least 85%, at least 90%, at least 95%, at least 96%, at least 97%, at least 98%, at least 99%, or 100% complementary to a RNA transcript sequence, or a portion thereof, encoded by the endogenous GA20 oxidase_3 or GA20 oxidase_5 gene, where the transcribable DNA sequence is operably linked to the endogenous Zm.SAMT gene promoter or a portion thereof. In another aspect, a GA20 oxidase_5 mutant allele comprises a transcribable DNA sequence operably linked to the endogenous Zm.SAMT gene promoter or a portion thereof, and at least 80%, at least 85%, at least 90%, at least 95%, at least 96%, at least 97%, at least 98%, at least 99%, or 100% complementary to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 1-3, 5-7, 9, and 11-38. In another aspect, a GA20 oxidase_5 mutant allele comprises a transcribable DNA sequence operably linked to the endogenous Zm.SAMT gene promoter or a portion thereof, and at least 80%, at least 85%, at least 90%, at least 95%, at least 96%, at least 97%, at least 98%, at least 99%, or 100% identical to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 1-3, 5-7, 10, and 39-66.

In an aspect, a GA20 oxidase_5 mutant allele comprises a genome modification comprising a deletion of at least 25, at least 30, at least 40, at least 50, at least 75, at least 100, at least 150, at least 200, at least 300, at least 400, at least 500, at least 750, at least 1000 consecutive nucleotides of the intergenic region between the endogenous GA20 oxidase_5 and SAMT genes.

In another aspect, a GA20 oxidase_5 mutant allele comprises a genome modification comprising a deletion of the entire intergenic region between the endogenous GA20 oxidase_5 and SAMT genes.

In an aspect, a GA20 oxidase_5 mutant allele comprises a genome modification comprising a deletion of one or more sequence elements selected from the group consisting of the 5′UTR, 1^{st} exon, 1^{st} intron, 2^{nd} exon, 2^{nd} intron, 3^{rd} exon, 3′ UTR, and any portion of the foregoing, of the endogenous GA20 oxidase_5 gene. In another aspect, a GA20 oxidase_5 mutant allele comprises a genome modification comprising a deletion of one or more sequence elements selected from the group consisting of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion of the foregoing, of the endogenous Zm.SAMT locus or gene.

In an aspect, a GA20 oxidase_5 mutant allele produces a RNA molecule comprising an antisense sequence that is at least 80%, at least 85%, at least 90%, at least 95%, at least 96%, at least 97%, at least 98%, at least 99%, or 100% complementary to a RNA transcript sequence, or a portion thereof, encoded by the endogenous GA20 oxidase_5 gene. In another aspect, a GA20 oxidase_5 mutant allele produces a RNA molecule comprising an antisense sequence that is at least 80%, at least 85%, at least 90%, at least 95%, at least 96%, at least 97%, at least 98%, at least 99%, or 100% complementary to a RNA transcript sequence that is at least 90%, at least 95%, at least 96%, at least 97%, at least 98%, at least 99%, or 100% identical to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 1-3, 5-7, 9, and 11-38.

In an aspect, a GA20 oxidase_5 mutant allele produces a RNA molecule comprising an antisense sequence that is at least 80%, at least 85%, at least 90%, at least 95%, at least 96%, at least 97%, at least 98%, at least 99%, or 100% complementary to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 1-3, 5-7, 9, and 11-38.

In an aspect, the present disclosure provides a modified corn plant, or plant part thereof, comprising a mutant allele of the endogenous GA20 oxidase_5 locus or gene, where the mutant allele comprises a genome modification which results in the production of an RNA molecule comprising an antisense sequence from a genomic segment of selected from the group consisting of an exon, a portion of an exon, an intron, a portion of an intron, a 5′ or 3′ untranslated region (UTR), a portion of an UTR, and any combination of the foregoing, of the endogenous GA20 oxidase_5 locus or gene. In another aspect, an antisense sequence can hybridize with a RNA transcript encoded by a wild-type or mutant allele of one or both of the endogenous GA20 oxidase_3 gene and the endogenous GA20 oxidase_5 gene. In a further aspect, the hybridization of an antisense sequence with a corresponding sense wild-type or mutant RNA transcript can suppress the expression of the wild-type allele of the endogenous GA20 oxidase_3 locus or gene, the wild-type allele of the endogenous GA20 oxidase_5 locus or gene, or both.

In an aspect, the present disclosure provides a modified corn plant, or plant part thereof, comprising a mutant allele of the endogenous GA20 oxidase_5 locus or gene, where the mutant allele comprises a genome modification which results in the transcription of at least a portion of the antisense strand of at least an exon, an intron, or an untranslated region (UTR) of the endogenous GA20 oxidase_5 gene, or any portion thereof.

In another aspect, the present disclosure provides a modified corn plant, or plant part thereof, comprising a mutant allele of the endogenous GA20 oxidase_5 locus or gene, where the mutant allele comprises the Zm.SAMT gene promoter, or a functional part thereof, operably linked to at least one transcribable antisense sequence of at least an exon, intron or untranslated region (UTR) of the endogenous GA20 oxidase_5 gene, or any portion thereof.

In a further aspect, the present disclosure provides a modified corn plant, or plant part thereof, comprising a mutant allele of the endogenous GA20 oxidase_5 locus or gene, where the mutant allele comprises one or more sequences selected from the group consisting of SEQ ID NOs: 87-105.

In a further aspect, the present disclosure provides a modified corn plant, or plant part thereof, comprising a mutant allele of the endogenous GA20 oxidase_5 locus or gene, wherein the mutant allele comprises a combination of deletion junction sequences as shown in individual plants listed in Table 5. Also provided are the GA20 oxidase_5 mutant alleles present in the individual R0/R1 plants listed in Table 5.

In an aspect, the present disclosure provides a modified corn plant, or plant part thereof, comprising a mutant allele of the endogenous GA20 oxidase_3 locus or gene, where the mutant allele comprises a first sequence and a second sequence; where the first sequence comprises one or more of the 5′ UTR, 1^{st} exon, 1^{st} intron, 2^{nd} exon, 2^{nd} intron, 3^{rd} exon, 3′ UTR, and/or any portion of the foregoing, of the endogenous Zm.GA20 oxidase_5 locus or gene; and where the second sequence comprises one or more of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and/or any portion of the foregoing, of the endogenous Zm.SAMT locus or gene; where the first sequence and the second sequence are contiguous or only separated by an intervening sequence of fewer than 550, fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.

In another aspect, the present disclosure provides a modified corn plant, or plant part thereof, comprising a mutant allele of the endogenous GA20 oxidase_5 locus or gene, where the mutant allele comprises a genomic deletion relative to a wild type allele of the endogenous GA20 oxidase_5 locus or gene, where the genomic deletion is flanked by a first sequence and a second sequence; where the first sequence comprises one or more of the 5′ UTR, 1^{st} exon, 1^{st} intron, 2^{nd} exon, 2^{nd} intron, 3^{rd} exon, 3′ UTR, and any portion of the foregoing, of the endogenous Zm.GA20 oxidase_5 locus or gene; and where the second sequence comprises one or more of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion of the foregoing, of the endogenous Zm.SAMT locus or gene.

In an aspect, a GA20 oxidase_5 mutant allele comprises a first sequence and a second sequence; where the first sequence comprises one or more of SEQ ID NOs: 11-18 and 59-66, or any portion thereof, and where the second sequence comprises one or more of SEQ ID NOs: 18-38 and 39-59, or any portion thereof.

In an aspect, a GA20 oxidase_5 mutant allele comprises a first sequence and a second sequence; where the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 11-18 and 59-66; where the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 18-38 and 39-59; where the first sequence and the second sequence are contiguous or only separated by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.

In an aspect, a GA20 oxidase_5 mutant allele comprises a genomic sequence comprising a first sequence and a second sequence; where the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 11-18 and 59-66; where the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 18-38 and 39-59; and where the genomic sequence is at least 50, at least 75, at least 100, at least 150, at least 200, at least 300, at least 400, at least 500, at least 750, at least 1000, at least 1500, at least 2000, at least 2500, at least 3000, at least 3500, at least 4000, at least 4500, or at least 5000, at least 5500, at least 6000, at least 6500, at least 7000, at least 7500, or at least 8000 consecutive nucleotides in length, and/or fewer than 9000, fewer than 8500, fewer than 8000, fewer than 7500, fewer than 7000, fewer than 6500, fewer than 6000, fewer than 5500, fewer than 5000, fewer than 4500, fewer than 4000, fewer than 3500, fewer than 3000, fewer than 2500, fewer than 2000, fewer than 1500, fewer than 1000, fewer than 750, fewer than 500, fewer than 250, fewer than 200, fewer than 150, fewer than 100, or fewer than 50 consecutive nucleotides in length. According to an aspect of the foregoing, the first sequence and the second sequence are contiguous or separated by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.

In an aspect, a GA20 oxidase_5 mutant allele comprises a first sequence and a second sequence; where the first sequence comprises one or more of SEQ ID NOs: 9-66, or any portion thereof, and where the second sequence comprises one or more of SEQ ID NOs: 9-66, or any portion thereof. In an aspect, a GA20 oxidase_5 mutant allele comprises a first sequence and a second sequence; where the first sequence comprises one or more of SEQ ID NOs: 9 and 11-38, or any portion thereof, and where the second sequence comprises one or more of SEQ ID NOs: 9 and 11-38, or any portion thereof. In an aspect, a GA20 oxidase_5 mutant allele comprises a first sequence and a second sequence; where the first sequence comprises one or more of SEQ ID NOs: 10 and 39-66, or any portion thereof, and where the second sequence comprises one or more of SEQ ID NOs: 10 and 39-66, or any portion thereof.

In an aspect, a GA20 oxidase_5 mutant allele comprises a first sequence and a second sequence; where the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 9-66, or of one or more of SEQ ID NOs: 9 and 11-38; where the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 9-66, or of one or more of SEQ ID NOs: 9 and 11-38; where the first sequence and the second sequence are contiguous or only separated by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.

In an aspect, a GA20 oxidase_5 mutant allele comprises a genomic sequence comprising a first sequence and a second sequence; where the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 9-66; where the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 9-66; and where the genomic sequence is at least 50, at least 75, at least 100, at least 150, at least 200, at least 300, at least 400, at least 500, at least 750, at least 1000, at least 1500, at least 2000, at least 2500, at least 3000, at least 3500, at least 4000, at least 4500, or at least 5000, at least 5500, at least 6000, at least 6500, at least 7000, at least 7500, or at least 8000 consecutive nucleotides in length, and/or fewer than 9000, fewer than 8500, fewer than 8000, fewer than 7500, fewer than 7000, fewer than 6500, fewer than 6000, fewer than 5500, fewer than 5000, fewer than 4500, fewer than 4000, fewer than 3500, fewer than 3000, fewer than 2500, fewer than 2000, fewer than 1500, fewer than 1000, fewer than 750, fewer than 500, fewer than 250, fewer than 200, fewer than 150, fewer than 100, or fewer than 50 consecutive nucleotides in length. According to an aspect of the foregoing, the first sequence and the second sequence are contiguous or separated by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.

In an aspect, a GA20 oxidase_5 mutant allele comprises a genomic deletion comprising a deletion of the intergenic region between the endogenous Zm.GA20 oxidase_5 locus or gene and the endogenous Zm.SAMT locus or gene. In another aspect, a GA20 oxidase_5 mutant allele comprises a genomic deletion having a length of at least 50, at least 100, at least 150, at least 200, at least 250, at least 500, at least 750, at least 1000, at least 1250, at least 1500, at least 2000, at least 3000, at least 4000, at least 5000, at least 6000, at least 7000, or at least 7500 nucleotides. In an aspect, a GA20 oxidase_5 mutant allele comprises a genomic deletion having a length of at most 1000, at most 1250, at most 1500, at most 2000, at most 3000, at most 4000, at most 5000, at most 6000, at most 7000, at most 7500, or at most 8000 nucleotides. In another aspect, a GA20 oxidase_5 mutant allele comprises a genomic deletion corresponding to a deletion of one or more genomic regions comprising a sequence selected from the group consisting of SEQ ID NOs: 11-66. As used herein, the phrase “at most” is intended to be synonymous with “less than or equal to.”

In an aspect, a GA20 oxidase_5 mutant allele comprises a genomic deletion which results in the production of an RNA transcript comprising an antisense sequence from a genomic segment of the endogenous GA20 oxidase_5 locus or gene selected from the group consisting of an exon, portion of an exon, an intron, portion of an intron, an untranslated region (UTR), portion of an UTR, and any combination of the foregoing. In another aspect, a GA20 oxidase_5 mutant allele can suppress the expression of a wild-type allele of the endogenous GA20 oxidase_3 locus or gene, a wild-type allele of the endogenous GA20 oxidase_5 locus or gene, or both.

In an aspect, a modified corn plant is homozygous for a mutant allele at the endogenous GA20 oxidase_5 locus or gene. In another aspect, a modified corn plant is heterozygous for the mutant allele at the endogenous GA20 oxidase_5 locus or gene. In a further aspect, a modified corn plant has a shorter plant height and/or improved lodging resistance relative to an unmodified control plant.

In an aspect, the present disclosure provides a method for producing a modified corn plant comprising a mutant allele of the endogenous GA20 oxidase_5 locus or gene, the method comprising: (a) generating two double-stranded breaks (DSB) in or near the endogenous GA20 oxidase_5 locus or gene in a corn cell using a targeted editing technique; and (b) regenerating or developing from the corn cell a corn plant, or plant part thereof, comprising a mutant allele of the endogenous GA20 oxidase_5 locus or gene, where the mutant allele comprises a genome modification deleting or disrupting the transcription termination sequence of the endogenous Zm.SAMT locus or gene. In another aspect, a method further comprises regenerating or developing a corn plant from the corn cell.

In another aspect, the present disclosure provides a method for producing a modified corn plant comprising a mutant allele of the endogenous GA20 oxidase_5 locus or gene, the method comprising: (a) generating a first and a second double-stranded breaks (DSB) in a corn cell using a targeted editing technique, where the first DSB is in a region selected from the group consisting of 5′ UTR, 1^{st} exon, 1^{st} intron, 2^{nd} exon, 2^{nd} intron, 3^{rd} exon, 3′ UTR, and any portion of the foregoing, of the endogenous GA20 oxidase_3 locus or gene, and the intergenic region between the endogenous Zm.GA20 oxidase_5 gene and the endogenous Zm.SAMT gene; where the second DSB is in a region selected from the group consisting of 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion of the foregoing, of the endogenous Zm.SAMT locus or gene, and the intergenic region between the endogenous Zm.GA20 oxidase_5 locus or gene and the endogenous Zm.SAMT locus or gene; (b) regenerating or developing from the corn cell a corn plant, or plant part thereof, comprising a genomic deletion, where the genomic deletion is flanked by the first DSB and the second DSB. In another aspect, a method further comprises regenerating or developing a corn plant from the corn cell.

In an aspect, a targeted editing technique used here comprises the use of at least one site-specific nuclease. In an aspect, a site-specific nuclease is selected from the group consisting of a zinc-finger nuclease, a meganuclease, an RNA-guided nuclease, a TALE-nuclease, a recombinase, a transposase, and any combination thereof. In another aspect, a site-specific nuclease is a RNA-guided nuclease selected from the group consisting of a Cas9 nuclease or a variant thereof, and a Cpf1 nuclease or a variant thereof.

In an aspect, a modified corn plant described here has a shorter plant height and/or improved lodging resistance relative to an unmodified control plant. In an aspect, a modified corn plant is at least 10%, at least 20%, at least 25%, at least 30%, at least 35%, or at least 40% shorter than an unmodified control plant. In another aspect, a modified corn plant has a stalk or stem diameter at one or more stem internodes is at least 5%, at least 10%, at least 15%, at least 20%, at least 25%, at least 30%, at least 35%, or at least 40% greater than the stalk or stem diameter at the same one or more internodes of an unmodified control plant. In an aspect, a modified corn plant has a stalk or stem diameter at one or more of the first, second, third, and/or fourth internode below the ear is at least 5%, at least 10%, at least 15%, at least 20%, at least 25%, at least 30%, at least 35%, or at least 40% greater than the same internode of an unmodified control plant. In another aspect, the level of one or more active GAs in at least one internode tissue of the stem or stalk of a modified corn plant is at least 5%, at least 10%, at least 15%, at least 20%, at least 25%, at least 30%, at least 35%, or at least 40% lower than the same internode tissue of an unmodified control plant. In an aspect, the level of one or more active GAs in at least one internode tissue of the stem or stalk of a modified corn plant is lower than the same internode tissue of an unmodified control plant.

In an aspect, a modified corn plant does not have any significant off-types in at least one female organ or ear. A modified corn plant may comprise at least one ear that is substantially free of male reproductive tissues or structures or other off-types. In an aspect, a modified corn plant exhibits essentially no reproductive abnormality or off-type—i.e., no significant or observable reproductive abnormality or off-type. In a further aspect, an off-type or reproductive abnormality is selected from the group consisting of male (tassel or anther) sterility, reduced kernel or seed number, and the presence of one or more masculinized or male (or male-like) reproductive structures in the female organ or ear (e.g., anther ear).

In another aspect, a modified corn plant comprises one or more traits, relative to an unmodified control plant, selected from the group consisting of shorter plant height, increased stalk/stem diameter, improved lodging resistance, reduced green snap, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and increased prolificacy.

In an aspect, a modified corn plant is an inbred. In another aspect, a modified corn plant is a hybrid.

According to further embodiments, methods are provided for transforming a plant cell, tissue or explant with a recombinant DNA molecule or construct encoding one or more molecules required for targeted genome editing (e.g., guide RNA(s) and/or site-directed nuclease(s)). Numerous methods for transforming chromosomes or plastids in a plant cell with a recombinant DNA molecule or construct are known in the art, which may be used according to method embodiments of the present invention to produce a transgenic plant cell and plant. Any suitable method or technique for transformation of a plant cell known in the art may be used according to present methods. Effective methods for transformation of plants include bacterially mediated transformation, such as Agrobacterium-mediated or Rhizobium-mediated transformation, and microprojectile or particle bombardment-mediated transformation. A variety of methods are known in the art for transforming explants with a transformation vector via bacterially mediated transformation or microprojectile or particle bombardment and then subsequently culturing, etc., those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art.

Methods of transforming plant cells and explants are well known by persons of ordinary skill in the art. Methods for transforming plant cells by microprojectile bombardment with particles coated with recombinant DNA are provided, for example, in U.S. Pat. Nos. 5,550,318; 5,538,880 6,160,208; 6,399,861; and 6,153,812, and Agrobacterium-mediated transformation is described, for example, in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any suitable method of plant transformation known or later developed in the art can be used to transform a plant cell or explant with any of the nucleic acid molecules, constructs or vectors provided herein.

Recipient cell(s) or explant or cellular targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, a callus cell, a chloroplast, a stomatal cell, a trichome cell, a root hair cell, a storage root cell, or a vascular tissue cell, a seed, embryo, meristem, cotyledon, hypocotyl, endosperm, root, shoot, stem, node, callus, cell suspension, protoplast, flower, leaf, pollen, anther, ovary, ovule, pericarp, bud, and/or vascular tissue, or any transformable portion of any of the foregoing. For plant transformation, any target cell(s), tissue(s), explant(s), etc., that may be used to receive a recombinant DNA transformation vector or molecule of the present disclosure may be collectively be referred to as an “explant” for transformation. Preferably, a transformable or transformed explant cell or tissue may be further developed or regenerated into a plant. Any cell or explant from which a fertile plant can be grown or regenerated is contemplated as a useful recipient cell or explant for practice of this disclosure (i.e., as a target explant for transformation). Callus can be initiated or created from various tissue sources, including, but not limited to, embryos or parts of embryos, non-embryonic seed tissues, seedling apical meristems, microspores, and the like. Any cells that are capable of proliferating as callus may serve as recipient cells for transformation. Transformation methods and materials for making transgenic plants (e.g., various media and recipient target cells or explants and methods of transformation and subsequent regeneration of into transgenic plants) are known in the art.

Transformation or editing of a target plant material or explant may be practiced in tissue culture on nutrient media, for example a mixture of nutrients that allow cells to grow in vitro or cell culture. Modified explants, cells or tissues may be subjected to additional culturing steps, such as callus induction, selection, regeneration, etc., as known in the art. Transformation or editing may also be carried out without creation or use of a callus tissue. Transformed or edited cells, tissues or explants containing a DNA sequence insertion or edit may be grown, developed or regenerated into transgenic plants in culture, plugs, or soil according to methods known in the art. Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the insertion. For example, a DNA sequence or inversion may be introduced into a first plant line that is amenable to transformation or editing, which may then be crossed with a second plant line to introgress the DNA sequence or edit (e.g., deletion) into the second plant line. Progeny of these crosses can be further back crossed into the desirable line multiple times, such as through 6 to 8 generations or back crosses, to produce a progeny plant with substantially the same genotype as the original parental line, but for the introduction of the DNA sequence or edit.

A modified plant, plant part, cell, or explant provided herein may be of an elite variety or an elite line. An elite variety or an elite line refers to a variety that has resulted from breeding and selection for superior agronomic performance. A modified (e.g., edited) plant, cell, or explant provided herein may be a hybrid plant, cell, or explant. As used herein, a “hybrid” is created by crossing two plants from different varieties, lines, inbreds, or species, such that the progeny comprises genetic material from each parent. Skilled artisans recognize that higher order hybrids can be generated as well. For example, a first hybrid can be made by crossing Variety A with Variety B to create a A×B hybrid, and a second hybrid can be made by crossing Variety C with Variety D to create an C×D hybrid. The first and second hybrids can be further crossed to create the higher order hybrid (A×B)×(C×D) comprising genetic information from all four parent varieties.

In an aspect, this disclosure provides a method for generating a corn plant comprising: (a) fertilizing at least one female corn plant with pollen from a male corn plant, wherein the female corn plant and/or the male corn plant comprises a mutant (e.g., edited) allele of the endogenous GA20 oxidase_5 locus or gene as provided herein, wherein the mutant allele comprises a genome modification comprising (i) a deletion of at least a portion of the transcription termination sequence of the endogenous Zm.SAMT gene, and where the mutant allele produces a RNA molecule comprising an antisense sequence complementary to all or part of the sense strand of the endogenous GA20 oxidase_5 gene; (ii) a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene; or (iii) a deletion of at least a portion of one or more of the following: 5′ UTR, 1^{st} exon, 1^{st} intron, 2^{nd} exon, 2^{nd} intron, 3^{rd} exon, 3′ UTR, and any portion thereof, and the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion thereof, of the endogenous Zm.SAMT gene; and (b) obtaining at least one seed produced by said fertilizing of step (a). According to an aspect, the at least one seed in step (b) comprises the mutant allele of the endogenous GA20 oxidase locus or gene from the female corn plant. In another aspect, the method further comprises (c) growing the at least one seed obtained in step (b) to generate at least one progeny corn plant comprising said mutant allele. In an aspect, the at least one progeny corn plant obtained in step (c) is heterozygous for the mutant allele. In an aspect, the at least one progeny corn plant obtained in step (c) is homozygous for the mutant allele. According to some aspects, such methods may further comprise (d) selecting at least one progeny corn plant that comprises the mutant allele. The corn plant selected in (d) can be either homozygous or heterozygous for the mutant allele.

In an aspect, the female corn plant is homozygous for a mutant allele. In another aspect, the female corn plant is heterozygous for the mutant allele. In an aspect, the male corn plant lacks the mutant allele. In an aspect, the male corn plant is heterozygous for the mutant allele. In an aspect, the male corn plant is homozygous for the mutant allele. In an aspect, the at least one progeny corn plant has a shorter plant height and/or improved lodging resistance relative to a control plant lacking the mutant allele. In an aspect, the at least one progeny corn plant has a shorter plant height and/or improved lodging resistance relative to the male or female corn plant. In an aspect, the female corn plant is an inbred corn plant. In an aspect, the female corn plant is a hybrid corn plant. In an aspect, the male corn plant is an inbred corn plant. In an aspect, the male corn plant is a hybrid corn plant. In an aspect, the female corn plant is an elite corn plant line. In an aspect, the male corn plant is an elite corn plant line. In an aspect, the female corn plant is a first inbred corn line or variety, and the male corn plant is of a different, second inbred corn line or variety. In an aspect, the female corn plant and the male corn plant are grown in a greenhouse or growth chamber. In an aspect, the female corn plant and the male corn plant are grown outdoors. In an aspect, the female corn plant and the male corn plant are grown in a field. In an aspect, the female corn plant has been detasseled. In an aspect, the female corn plant is a cytoplasmically male sterile corn plant.

As used herein, “detasseled” corn refers to corn where the pollen-producing flowers, or tassels, have been removed. Detasseling is typically performed before the tassel can shed pollen. Detasseling can be accomplished via machine detasseling, manual detasseling, or a combination of both machine and manual detasseling. Detasseling removes the uppermost leaves of the corn plant along with the developing tassel. Detasseled corn plants retain their female flowers, which may be pollinated by pollen from another corn plant and eventually produce kernels on the ear. In an aspect, a corn plant provided herein is a detasseled corn plant.

As an alternative to chemical treatment, corn plants (or female corn plants) can be made male sterile through genetic crosses and inheritance causing cytoplasmic male sterility. As used herein, the term “cytoplasmic male sterility” or “CMS” refers to a condition where a corn plant is partially or fully incapable of producing functional pollen. As known in the art, cytoplasmic male sterility is a maternally inherited trait that is commonly associated with unusual open reading frames within the mitochondrial genome which cause cytoplasmic dysfunction. In an aspect, a corn plant or female corn plant provided herein is a cytoplasmic male sterile corn plant.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. In some embodiments, a vector or polynucleotide provided herein comprises at least one selectable marker gene selected from the group consisting of nptII, aph IV, aadA, aac3, aacC4, bar, pat, DMO, EPSPS, aroA, GFP, and GUS. Plant transformation may also be carried out in the absence of selection during one or more steps or stages of culturing, developing or regenerating transformed explants, tissues, plants and/or plant parts.

According to present embodiments, methods for transforming a plant cell, tissue or explant with a recombinant DNA molecule or construct may further include site-directed or targeted integration. According to these methods, a portion of a recombinant DNA donor template molecule (i.e., an insertion sequence) may be inserted or integrated at a desired site or locus within the plant genome. The insertion sequence of the donor template may comprise a transgene or construct, such as a transgene or transcribable DNA sequence of interest that encodes an anti-sense RNA sequence targeting an endogenous GA oxidase gene for suppression. The donor template may also have one or two homology arms flanking the insertion sequence to promote the targeted insertion through homologous recombination and/or homology-directed repair. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a monocot or cereal plant (e.g., a corn plant). Thus, a recombinant DNA molecule of the present disclosure may comprise a donor template for site-directed or targeted integration of a transgene or construct, such as a transgene or transcribable DNA sequence of interest that encodes an anti-sense RNA sequence targeting an endogenous GA oxidase gene for suppression, into the genome of a plant. In an aspect, this disclosure provides a recombinant DNA construct comprising one or more donor templates. In an aspect, a recombinant DNA construct comprising one or more donor templates can be introduced to a plant cell, plant tissue or plant part provided herein using any plant transformation technique known in the art.

Any site or locus within the genome of a plant may potentially be chosen for site-directed integration of a transgene, construct or transcribable DNA sequence provided herein. For site-directed integration, a double-strand break (DSB) or nick may first be made at a selected genomic locus with a site-specific nuclease, such as, for example, a zinc-finger nuclease, an engineered or native meganuclease, a TALE-endonuclease, or an RNA-guided endonuclease (e.g., Cas9 or Cpf1). Any method known in the art for site-directed integration may be used. In the presence of a donor template molecule with an insertion sequence, the DSB or nick may then be repaired by homologous recombination between homology arm(s) of the donor template and the plant genome, or by non-homologous end joining (NHEJ), resulting in site-directed integration of the insertion sequence into the plant genome to create the targeted insertion at the site of the DSB or nick. Thus, site-specific insertion or integration of a transgene, construct or sequence may be achieved.

A site-specific nuclease provided herein may be selected from the group consisting of a zinc-finger nuclease (ZFN), a meganuclease, an RNA-guided endonuclease, a TALE-endonuclease (TALEN), a recombinase, a transposase, or any combination thereof. See, e.g., Khandagale, K. et al., “Genome editing for targeted improvement in plants,” Plant Biotechnol Rep 10: 327-343 (2016); and Gaj, T. et al., “ZFN, TALEN and CRISPR/Cas-based methods for genome engineering,” Trends Biotechnol. 31(7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp1 recombinase. According to some embodiments, a Cre recombinase or a Gin recombinase provided herein is tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator.

According to embodiments of the present disclosure, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (or Cas12a), CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cpf1 (or Cas12a) enzyme.

In an aspect, a site-specific nuclease provided herein is selected from the group consisting of a zinc-finger nuclease, a meganuclease, an RNA-guided nuclease, a TALE-nuclease, a recombinase, a transposase, or any combination thereof. In another aspect, a site-specific nuclease provided herein is selected from the group consisting of a Cas9 or a Cpf1 (or Cas12a). In another aspect, a site-specific nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, an RNA-guided nuclease provided herein is selected from the group consisting of a Cas9 or a Cpf1 (or Cas12a). In another aspect, an RNA guided nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1 (or Cas12a), CasX, CasY, a homolog thereof, or a modified version thereof. In another aspect, a method and/or a composition provided herein comprises at least one, at least two, at least three, at least four, at least five, at least six, at least seven, at least eight, at least nine, or at least ten site-specific nucleases. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific nucleases.

For RNA-guided endonucleases, a guide RNA (gRNA) molecule is further provided to direct the endonuclease to a target site in the genome of the plant via base-pairing or hybridization to cause a DSB or nick at or near the target site. The gRNA may be transformed or introduced into a plant cell or tissue (perhaps along with a nuclease, or nuclease-encoding DNA molecule, construct or vector) as a gRNA molecule, or as a recombinant DNA molecule, construct or vector comprising a transcribable DNA sequence encoding the guide RNA operably linked to a plant-expressible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a GA oxidase gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al., “Target specificity of the CRISPR-Cas9 system,” Quant Biol. 2(2): 59-70 (2014), the content and disclosure of which is incorporated herein by reference. The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

According to some embodiments, a recombinant DNA construct or vector may comprise a first polynucleotide sequence encoding a site-specific nuclease and a second polynucleotide sequence encoding a guide RNA that may be introduced into a plant cell together via plant transformation techniques. Alternatively, two recombinant DNA constructs or vectors may be provided including a first recombinant DNA construct or vector and a second DNA construct or vector that may be introduced into a plant cell together or sequentially via plant transformation techniques, wherein the first recombinant DNA construct or vector comprises a polynucleotide sequence encoding a site-specific nuclease and the second recombinant DNA construct or vector comprises a polynucleotide sequence encoding a guide RNA. According to some embodiments, a recombinant DNA construct or vector comprising a polynucleotide sequence encoding a site-specific nuclease may be introduced via plant transformation techniques into a plant cell that already comprises (or is transformed with) a recombinant DNA construct or vector comprising a polynucleotide sequence encoding a guide RNA. Alternatively, a recombinant DNA construct or vector comprising a polynucleotide sequence encoding a guide RNA may be introduced via plant transformation techniques into a plant cell that already comprises (or is transformed with) a recombinant DNA construct or vector comprising a polynucleotide sequence encoding a site-specific nuclease. According to yet further embodiments, a first plant comprising (or transformed with) a recombinant DNA construct or vector comprising a polynucleotide sequence encoding a site-specific nuclease may be crossed with a second plant comprising (or transformed with) a recombinant DNA construct or vector comprising a polynucleotide sequence encoding a guide RNA. Such recombinant DNA constructs or vectors may be transiently transformed into a plant cell or stably transformed or integrated into the genome of a plant cell.

In an aspect, vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, vectors comprising polynucleotides encoding a Cas9 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous GA oxidase gene of a corn plant, such as the GA20 oxidase_3 gene or the GA20 oxidase_5 gene in corn, to create a DSB or nick at such genomic locus to knockout or knockdown expression of the GA oxidase gene via repair of the DSB or nick. For example, an engineered site-specific nuclease, such as a recombinase, zinc finger nuclease (ZFN), meganuclease, or TALEN, may be designed to target and bind to (i) a target site within the genome of a plant corresponding to a sequence within SEQ ID NO: 1, or its complementary sequence, to create a DSB or nick at the genomic locus for the GA20 oxidase_3 gene, or (ii) a target site within the genome of a plant corresponding to a sequence within SEQ ID NO: 5, or its complementary sequence, to create a DSB or nick at the genomic locus for the GA20 oxidase_5 gene, which may then lead to the creation of a mutation or insertion of a sequence at the site of the DSB or nick, through cellular repair mechanisms, which may be guided by a donor molecule or template.

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp1 recombinase. In an aspect, a Cre recombinase or a Gin recombinase provided herein may be tethered to a zinc-finger DNA binding domain. The Flp-FRT site-directed recombination system may come from the 2μ plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., U.S. patent application Nos. 2005/0064474, 2009/0117617, and 2012/0142062, the contents and disclosures of which are incorporated herein by reference. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory, because the DNA-binding specificities of zinc finger domains can be re-engineered using one of various methods, customized ZFNs can theoretically be constructed to target nearly any target sequence (e.g., at or near a GA oxidase gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick. In an aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection, or Agrobacterium-mediated transformation). The ZFNs may be introduced as ZFN proteins, as polynucleotides encoding ZFN proteins, and/or as combinations of proteins and protein-encoding polynucleotides.

Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SceI, I-AniI, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a GA oxidase gene. In an aspect, a method and/or composition provided herein comprises one or more, two or more, three or more, four or more, or five or more meganucleases. In another aspect, a meganuclease provided herein is capable of generating a targeted DSB. In an aspect, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

TALENs are artificial restriction enzymes generated by fusing the transcription activator-like effector (TALE) DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In some aspects, the nuclease is selected from a group consisting of PvuII, MutH, TevI, FokI, AlwI, MlyI, SW, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also refers to one or both members of a pair of TALENs that work together to cleave DNA at the same site.

Transcription activator-like effectors (TALEs) can be engineered to bind practically any DNA sequence, such as at or near the genomic locus of a GA oxidase gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122; Cermak et al., Nucleic Acids Research (2011). 39:e82; and tale-nt.cac.cornell.edu/about. In an aspect, a method and/or composition provided herein comprises one or more, two or more, three or more, four or more, or five or more TALENs. In another aspect, a TALEN provided herein is capable of generating a targeted DSB. In an aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation). See, e.g., U.S. patent application Nos. 2011/0145940, 2011/0301073, and 2013/0117869, the contents and disclosures of which are incorporated herein by reference.

Embodiments of the present disclosure further include methods for making or producing modified plants described herein, such as by transformation, genome editing, mutating, crossing, etc., wherein the method comprises introducing a recombinant DNA molecule, construct or sequence of interest into a plant cell, or editing or mutating the genomic locus of an endogenous GA oxidase gene, and then regenerating or developing the modified plant from the transformed or edited plant cell, which may be performed under selection pressure. Such methods may comprise transforming a plant cell with a recombinant DNA molecule, construct or sequence of interest, and selecting for a plant having one or more altered phenotypes or traits, such as one or more of the following traits at one or more stages of development: shorter or semi-dwarf stature or plant height, shorter internode length in one or more internode(s), increased stalk/stem diameter, improved lodging resistance, reduced green snap, deeper roots, increased leaf area, earlier canopy closure, increased foliar water content and/or higher stomatal conductance under water limiting conditions, reduced anthocyanin content and/or area in leaves under normal or nitrogen or water limiting stress conditions, improved yield-related traits including a larger female reproductive organ or ear, an increase in ear weight, harvest index, yield, seed or kernel number, and/or seed or kernel weight, increased stress tolerance, such as increased drought tolerance, increased nitrogen utilization, and/or increased tolerance to high density planting, as compared to a wild type or control plant.

According to another aspect of the present disclosure, methods are provided for planting a modified plant(s) provided herein at a normal/standard or high density in field. According to some embodiments, the yield of a crop plant per acre (or per land area) may be increased by planting a modified plant(s) of the present disclosure at a higher density in the field. As described herein, modified plants having a genome-edited GA oxidase gene, may have reduced plant height, shorter internode(s), increased stalk/stem diameter, and/or increased lodging resistance. It is proposed that modified plants may tolerate high density planting conditions since an increase in stem diameter may resist lodging and the shorter plant height may allow for increased light penetrance to the lower leaves under high density planting conditions. Thus, modified plants provided herein may be planted at a higher density to increase the yield per acre (or land area) in the field. For row crops, higher density may be achieved by planting a greater number of seeds/plants per row length and/or by decreasing the spacing between rows.

According to some embodiments, a modified crop plant may be planted at a density in the field (plants per land/field area) that is at least 5%, 10%, 15%, 20%, 25%, 50%, 75%, 100%, 125%, 150%, 175%, 200%, 225%, or 250% higher than the normal planting density for that crop plant according to standard agronomic practices. A modified crop plant may be planted at a density in the field of at least 38,000 plants per acre, at least 40,000 plants per acre, at least 42,000 plants per acre, at least 44,000 plants per acre, at least 45,000 plants per acre, at least 46,000 plants per acre, at least 48,000 plants per acre, 50,000 plants per acre, at least 52,000 plants per acre, at least 54,000 per acre, or at least 56,000 plants per acre. As an example, corn plants may be planted at a higher density, such as in a range from about 38,000 plants per acre to about 60,000 plants per acre, or about 40,000 plants per acre to about 58,000 plants per acre, or about 42,000 plants per acre to about 58,000 plants per acre, or about 40,000 plants per acre to about 45,000 plants per acre, or about 45,000 plants per acre to about 50,000 plants per acre, or about 50,000 plants per acre to about 58,000 plants per acre, or about 52,000 plants per acre to about 56,000 plants per acre, or about 38,000 plants per acre, about 42,000 plant per acre, about 46,000 plant per acre, or about 48,000 plants per acre, about 50,000 plants per acre, or about 52,000 plants per acre, or about 54,000 plant per acre, as opposed to a standard density range, such as about 18,000 plants per acre to about 38,000 plants per acre.

The height of a corn plant can be measured using a variety of methods known in the art. which may be based on a variety of anatomical locations on a corn plant. In an aspect, the height of a corn plant is measured as the distance between the soil or ground and the ligule (or collar) of the uppermost fully-expanded leaf of the corn plant. As used herein, a “fully-expanded leaf” is a leaf where the leaf blade is exposed and both the ligule and auricle are visible at the blade/sheath boundary. In another aspect, the height of a corn plant is measured as the distance between the soil or ground and the upper leaf surface of the leaf farthest from the soil or ground. In another aspect, the height of a corn plant is measured as the distance between the soil or ground and the arch of the highest corn leaf that is at least 50% developed. As used herein, an “arch of the highest corn leaf” is the highest point of the arch of the uppermost leaf of the corn plant that is curving downward. In another aspect, the height of a corn plant is measured at the first reproductive (R1) stage. Exemplary, non-limiting methods of measuring plant height include comparing photographs of corn plants to a height reference, or physically measuring individual corn plants with a suitable ruler, stick, or measuring device. Unless otherwise specified, corn plant heights are mature or full-growth plant heights measured at a reproductive or late vegetation stage. Those in the art recognize that, when comparing a modified corn plant to a control corn plant, the measurements must be made at the same stage of growth. It would be improper, as a non-limiting example, to compare the height of a modified corn plant at R3 stage to the height of a control corn plant at V6 stage, even if both plants had been growing for the same amount of time. Unless otherwise specified, plant height is measured at R2 growth stage from the soil level to the base of the uppermost fully expanded leaf.

As used herein, the term “ground” or “ground level” used in relation to a corn plant, such as to measure plant height, refers to the top or uppermost surface of the growth medium or soil (e.g., earth) from which the corn plant grows.

Corn plant height varies depending on the line or variety grown, whether the plant is a hybrid or inbred, and environmental conditions. Although hybrid corn plants can reach a height of over 3.6 meters tall by maturity, a height of around 2.0-2.5 meters by maturity for hybrid plants is more common. Modified corn plants provided herein have a reduced plant height comparted to a control plant, such as less than 2.0 meters, less than 1.9 meters, less than 1.8 meters, less than 1.7 meters, less than 1.6 meters, or less than 1.5 meters.

According to embodiments of the present disclosure, a modified corn plant(s) is/are provided that comprise (i) a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and/or (ii) an average stem or stalk diameter of at least 18 mm, at least 18.5 mm, at least 19 mm, at least 19.5 mm, at least 20 mm, at least 20.5 mm, at least 21 mm, at least 21.5 mm, or at least 22 mm. Stated a different way, a modified corn plant(s) is/are provided that comprise a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and/or an average stem or stalk diameter that is greater than 18 mm, greater than 18.5 mm, greater than 19 mm, greater than 19.5 mm, greater than 20 mm, greater than 20.5 mm, greater than 21 mm, greater than 21.5 mm, or greater than 22 mm. Any such plant height trait or range that is expressed in millimeters (mm) may be converted into a different unit of measurement based on known conversions (e.g., one inch is equal to 2.54 cm or 25.4 millimeters, and millimeters (mm), centimeters (cm) and meters (m) only differ by one or more powers of ten). Thus, any measurement provided herein is further described in terms of any other comparable units of measurement according to known and established conversions. However, the exact plant height and/or stem diameter of a modified corn plant may depend on the environment and genetic background. Thus, the change in plant height and/or stem diameter of a modified corn plant may instead be described in terms of a minimum difference or percent change relative to a control plant. A modified corn plant may further comprise at least one ear that is substantially free of male reproductive tissues or structures or other off-types.

According to embodiments of the present disclosure, modified corn plants are provided that comprise a plant height during late vegetative and/or reproductive stages of development (e.g., at R3 stage) of between 1000 mm and 1800 mm, between 1000 mm and 1700 mm, between 1050 mm and 1700 mm, between 1100 mm and 1700 mm, between 1150 mm and 1700 mm, between 1200 mm and 1700 mm, between 1250 mm and 1700 mm, between 1300 mm and 1700 mm, between 1350 mm and 1700 mm, between 1400 mm and 1700 mm, between 1450 mm and 1700 mm, between 1000 mm and 1500 mm, between 1050 mm and 1500 mm, between 1100 mm and 1500 mm, between 1150 mm and 1500 mm, between 1200 mm and 1500 mm, between 1250 mm and 1500 mm, between 1300 mm and 1500 mm, between 1350 mm and 1500 mm, between 1400 mm and 1500 mm, between 1450 mm and 1500 mm, between 1000 mm and 1600 mm, between 1100 mm and 1600 mm, between 1200 mm and 1600 mm, between 1300 mm and 1600 mm, between 1350 mm and 1600 mm, between 1400 mm and 1600 mm, between 1450 mm and 1600 mm, of between 1000 mm and 2000 mm, between 1200 mm and 2000 mm, between 1200 mm and 1800 mm, between 1300 mm and 1700 mm, between 1400 mm and 1700 mm, between 1400 mm and 1600 mm, between 1400 mm and 1700 mm, between 1400 mm and 1800 mm, between 1400 mm and 1900 mm, between 1400 mm and 2000 mm, or between 1200 mm and 2500 mm, and/or an average stem diameter of between 17.5 mm and 22 mm, between 18 mm and 22 mm, between 18.5 and 22 mm, between 19 mm and 22 mm, between 19.5 mm and 22 mm, between 20 mm and 22 mm, between 20.5 mm and 22 mm, between 21 mm and 22 mm, between 21.5 mm and 22 mm, between 17.5 mm and 21 mm, between 17.5 mm and 20 mm, between 17.5 mm and 19 mm, between 17.5 mm and 18 mm, between 18 mm and 21 mm, between 18 mm and 20 mm, or between 18 mm and 19 mm. A modified corn plant may be substantially free of off-types, such as male reproductive tissues or structures in one or more ears of the modified corn plant.

According to embodiments of the present disclosure, modified corn plants are provided that have (i) a plant height that is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the height of a wild-type or control plant, and/or (ii) a stem or stalk diameter that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the stem diameter of the wild-type or control plant. According to embodiments of the present disclosure, a modified corn plant may have a reduced plant height that is no more than 20%, 25%, 30%, 35%, 40%, 45%, 50%, 55%, or 60% shorter than the height of a wild-type or control plant, and/or a stem or stalk diameter that is less than (or not more than) 10%, 15%, 20%, 25%, 30%, 35%, 40%, 45%, 50%, 55%, 60%, 65%, 70%, 75%, 80%, 85%, 90%, 95%, or 100% greater than the stem or stalk diameter of a wild-type or control plant. For example, a modified plant may have (i) a plant height that is at least 10%, at least 15%, or at least 20% less or shorter (i.e., greater than or equal to 10%, 15%, or 20% shorter), but not greater or more than 50% shorter, than a wild type or control plant, and/or (ii) a stem or stalk diameter that is that is at least 5%, at least 10%, or at least 15% greater, but not more than 30%, 35%, or 40% greater, than a wild type or control plant. For clarity, the phrases “at least 20% shorter” and “greater than or equal to 20% shorter” would exclude, for example, 10% shorter. Likewise for clarity, the phrases “not greater than 50% shorter”, “no more than 50% shorter” and “not more than 50% shorter” would exclude 60% shorter; the phrase “at least 5% greater” would exclude 2% greater; and the phrases “not more than 30% greater” and “no more than 30% greater” would exclude 40% greater.

According to embodiments of the present disclosure, modified corn plants are provided that comprise a height between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the height of a wild-type or control plant, and/or a stem or stalk diameter that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, between 50% and 75%, between 8% and 20%, or between 8% and 15% greater than the stem or stalk diameter of the wild-type or control plant.

As used herein, “internode length” refers to the distance between two consecutive internodes on the stem of a plant. According to embodiments of the present disclosure, modified corn plants are provided that comprise an average internode length (or a minus-2 internode length and/or minus-4 internode length relative to the position of the ear) that is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the same or average internode length of a wild-type or control plant. The “minus-2 internode” of a corn plant refers to the second internode below the ear of the plant, and the “minus-4 internode” of a corn plant refers to the fourth internode below the ear of the plant According to many embodiments, modified corn plants are provided that have an average internode length (or a minus-2 internode length and/or minus-4 internode length relative to the position of the ear) that is between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the same or average internode length of a wild-type or control plant.

According to embodiments of the present disclosure, modified corn plants are provided that comprise an ear weight (individually or on average) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the ear weight of a wild-type or control plant. A modified corn plant provided herein may comprise an ear weight that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 25% and 75%, between 25% and 50%, or between 50% and 75% greater than the ear weight of a wild-type or control plant.

According to embodiments of the present disclosure, modified corn plants are provided that have a harvest index of at least 0.57, at least 0.58, at least 0.59, at least 0.60, at least 0.61, at least 0.62, at least 0.63, at least 0.64, or at least 0.65 (or greater). A modified corn plant may comprise a harvest index of between 0.57 and 0.65, between 0.57 and 0.64, between 0.57 and 0.63, between 0.57 and 0.62, between 0.57 and 0.61, between 0.57 and 0.60, between 0.57 and 0.59, between 0.57 and 0.58, between 0.58 and 0.65, between 0.59 and 0.65, or between 0.60 and 0.65. A modified corn plant may have a harvest index that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, or at least 50% greater than the harvest index of a wild-type or control plant. A modified corn plant may have a harvest index that is between 1% and 45%, between 1% and 40%, between 1% and 35%, between 1% and 30%, between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 30%, or between 5% and 40% greater than the harvest index of a wild-type or control plant.

According to embodiments of the present disclosure, modified corn plants are provided that have an increase in harvestable yield of at least 1 bushel per acre, at least 2 bushels per acre, at least 3 bushels per acre, at least 4 bushels per acre, at least 5 bushels per acre, at least 6 bushels per acre, at least 7 bushels per acre, at least 8 bushels per acre, at least 9 bushels per acre, or at least 10 bushels per acre, relative to a wild-type or control plant. A modified corn plant may have an increase in harvestable yield between 1 and 10, between 1 and 8, between 2 and 8, between 2 and 6, between 2 and 5, between 2.5 and 4.5, or between 3 and 4 bushels per acre. A modified corn plant may have an increase in harvestable yield that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, or at least 25% greater than the harvestable yield of a wild-type or control plant. A modified corn plant may have a harvestable yield that is between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 25%, between 2% and 10%, between 2% and 9%, between 2% and 8%, between 2% and 7%, between 2% and 6%, between 2% and 5%, or between 2% and 4% greater than the harvestable yield of a wild-type or control plant.

According to embodiments of the present disclosure, a modified corn plant is provided that has a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a wild-type or control plant. A modified corn plant may have a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a wild-type or control plant. Further provided are populations of corn plants having increased lodging resistance and a reduced lodging frequency. Populations of modified corn plants are provided having a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a population of wild-type or control plants. A population of modified corn plants may comprise a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a population of wild-type or control plants, which may be expressed as an average over a specified number of plants or crop area of equal density.

According to embodiments of the present disclosure, modified corn plants are provided having a significantly reduced or decreased plant height (e.g., 2000 mm or less) and a significantly increased stem diameter (e.g., 18 mm or more), relative to a wild-type or control plant. According to these embodiments, the decrease or reduction in plant height and increase in stem diameter may be within any of the height, diameter or percentage ranges recited herein. Modified corn plants having a significantly reduced plant height and/or a significantly increased stem diameter relative to a wild-type or control plant may further have at least one ear that is substantially free of male reproductive tissues or structures and/or other off-types. The non-coding RNA molecule may be a miRNA, siRNA, or miRNA or siRNA precursor molecule. According to some embodiments, modified corn plants having a significantly reduced plant height and/or an increased stem diameter relative to a wild-type or control plant may further have an increased harvest index and/or increased lodging resistance relative to the wild-type or control plant.

According to embodiments of the present invention, modified corn plants are provided having a reduced gibberellin content (in active form) in at least the stem and internode tissue(s), such as the stem, internode, leaf and/or vascular tissue(s), as compared to the same tissue(s) of wild-type or control plants. According to many embodiments, modified corn plants are provided having a significantly reduced plant height and/or a significantly increased stem diameter relative to wild-type or control plants, wherein the modified corn plants further have significantly reduced or decreased level(s) of active gibberellins or active GAs (e.g., one or more of GA1, GA3, GA4, and/or GA7) in one or more stem, internode, leaf and/or vascular tissue(s), relative to the same tissue(s) of the wild-type or control plants. For example, the level of one or more active GAs in the stem, internode, leaf and/or vascular tissue(s) of a modified corn plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less or lower than in the same tissue(s) of a wild-type or control corn plant.

According to some embodiments, a modified corn plant may comprise an active gibberellin (GA) level(s) (e.g., one or more of GA1, GA3, GA4, and/or GA7) in one or more stem, internode, leaf and/or vascular tissue(s) that is between 5% and 50%, between 10% and 100%, between 20% and 100%, between 30% and 100%, between 40% and 100%, between 50% and 100%, between 60% and 100%, between 70% and 100%, between 80% and 100%, between 80% and 90%, between 10% and 90%, between 10% and 80%, between 10% and 70%, between 10% and 60%, between 10% and 50%, between 10% and 40%, between 10% and 30%, between 10% and 20%, between 50% and 100%, between 20% and 90%, between 20% and 80%, between 20% and 70%, between 20% and 60%, between 20% and 50%, between 20% and 40%, between 20% and 40%, between 20% and 30%, between 30% and 90%, between 30% and 80%, between 30% and 70%, between 30% and 60%, between 30% and 50%, between 30% and 40%, between 40% and 90% between 40% and 80%, between 40% and 70%, between 40% and 60%, between 40% and 50%, between 50% and 90%, between 50% and 80%, between 50% and 70%, between 50% and 60%, between 60% and 90%, between 60% and 80%, between 60% and 70%, between 70% and 90%, or between 70% and 80% less or (or lower) than in the same tissue(s) of a wild-type or control corn plant. A modified corn plant having a reduced active gibberellin (GA) level(s) in one or more stem, internode, leaf and/or vascular tissue(s) may further be substantially free of off-types, such as male reproductive tissues or structures and/or other off-types in at least one ear of a modified corn plant.

According to embodiments of the present disclosure, modified corn plants are provided having a significantly reduced or eliminated expression level of one or more GA20 oxidase gene transcript(s) and/or protein(s) in one or more tissue(s), such as one or more stem, internode, leaf and/or vascular tissue(s), of the modified plants, as compared to the same tissue(s) of wild-type or control plants. According to many embodiments, a modified corn plant is provided comprising a significantly reduced plant height and/or a significantly increased stem diameter relative to wild-type or control plants, wherein the modified corn plant has a significantly reduced or eliminated expression level of one or more GA20 oxidase gene transcript(s) and/or protein(s) in one or more tissues, such as one or more stem, internode, leaf and/or vascular tissue(s), of the modified plant, as compared to the same tissue(s) of a wild-type or control corn plant. For example, a modified corn plant has a significantly reduced or eliminated expression level of a GA20 oxidase_3 and/or GA20 oxidase_5 gene transcript(s) and/or protein(s), in the whole modified plant, or in one or more stem, internode, leaf and/or vascular tissue(s) of the modified plant, as compared to the same tissue(s) of a wild-type or control plant. For example, the level of one or more GA20 oxidase gene transcript(s) and/or protein(s), or one or more GA oxidase (or GA oxidase-like) gene transcript(s) and/or protein(s), in one or more stem, internode, leaf and/or vascular tissue(s) of a modified corn plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less or lower than in the same tissue(s) of a wild-type or control corn plant.

According to some embodiments, a modified corn plant may comprise level(s) of one or more GA20 oxidase gene transcript(s) and/or protein(s), or one or more GA oxidase (or GA oxidase-like) gene transcript(s) and/or protein(s), in the whole plant, or in one or more stem, internode, leaf and/or vascular tissue(s), that is between 5% and 50%, between 10% and 100%, between 20% and 100%, between 30% and 100%, between 40% and 100%, between 50% and 100%, between 60% and 100%, between 70% and 100%, between 80% and 100%, between 80% and 90%, between 10% and 90%, between 10% and 80%, between 10% and 70%, between 10% and 60%, between 10% and 50%, between 10% and 40%, between 10% and 30%, between 10% and 20%, between 50% and 100%, between 20% and 90%, between 20% and 80%, between 20% and 70%, between 20% and 60%, between 20% and 50%, between 20% and 40%, between 20% and 40%, between 20% and 30%, between 30% and 90%, between 30% and 80%, between 30% and 70%, between 30% and 60%, between 30% and 50%, between 30% and 40%, between 40% and 90% between 40% and 80%, between 40% and 70%, between 40% and 60%, between 40% and 50%, between 50% and 90%, between 50% and 80%, between 50% and 70%, between 50% and 60%, between 60% and 90%, between 60% and 80%, between 60% and 70%, between 70% and 90%, or between 70% and 80% less or lower than in the same tissue(s) of a wild-type or control corn plant. A modified corn plant having a reduced or eliminated expression level of at least one GA20 oxidase gene(s) in one or more tissue(s), may also be substantially free of off-types, such as male reproductive tissues or structures and/or other off-types in at least one ear of the modified corn plant.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. Nucleic acids can be isolated and detected using techniques known in the art. For example, nucleic acids can be isolated and detected using, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis. Any method known in the art may be used to screen for, and/or identify, cells, plants, etc., having a transgene or genome edit in its genome, which may be based on any suitable form of visual observation, selection, molecular technique, etc.

In some embodiments, methods are provided for detecting recombinant nucleic acids and/or polypeptides in plant cells. For example, nucleic acids may be detected using hybridization probes or through production of amplicons using PCR with primers as known in the art. Hybridization between nucleic acids is discussed in Sambrook et al. (1989, Molecular Cloning: A Laboratory Manual, 2^{nd} Ed., Cold Spring Harbor Laboratory Press, Cold Spring Harbor, N.Y.). Polypeptides can be detected using antibodies. Techniques for detecting polypeptides using antibodies include enzyme linked immunosorbent assays (ELISAs), Western blots, immunoprecipitations, immunofluorescence, and the like. An antibody provided herein may be a polyclonal antibody or a monoclonal antibody. An antibody having specific binding affinity for a polypeptide provided herein can be generated using methods known in the art. An antibody or hybridization probe may be attached to a solid support, such as a tube, plate or well, using methods known in the art.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials.

The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

The following non-limiting embodiments are envisioned:
1. A modified corn plant, or plant part thereof, comprising a mutant allele of the endogenous GA20 oxidase_5 locus, wherein the mutant allele comprises a genome modification comprising a deletion of at least a portion of the transcription termination sequence of the endogenous Zm.SAMT gene, and wherein the mutant allele produces a RNA molecule comprising an antisense sequence complementary to all or part of the sense strand of the endogenous GA20 oxidase_5 gene.
2. A modified corn plant, or plant part thereof, comprising a mutant allele of the endogenous GA20 oxidase_5 locus, wherein the mutant allele comprises a genome modification comprising a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene.
3. A modified corn plant, or plant part thereof, comprising a mutant allele of the endogenous GA20 oxidase_5 locus, wherein the mutant allele comprises a genome modification comprising a deletion of at least a portion of one or more of the following: 5′ UTR, 1^{st} exon, 1^{st} intron, 2^{nd} exon, 2^{nd} intron, 3^{rd} exon, 3′ UTR, and any portion thereof, and the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion thereof, of the endogenous Zm.SAMT gene.
4. The modified corn plant, or plant part thereof, of any one of embodiments 1-3, wherein the mutant allele comprises the endogenous Zm.SAMT gene promoter, or a portion thereof, operably linked to a transcribable DNA sequence encoding a RNA molecule that causes suppression of one or both of the endogenous GA20 oxidase_3 gene and the endogenous GA20 oxidase_5 gene.
5. The modified corn plant, or plant part thereof, of any one of embodiments 1-3, wherein the mutant allele comprises the endogenous Zm.SAMT gene promoter, or a portion thereof, operably linked to a transcribable DNA sequence encoding a RNA molecule comprising an antisense sequence that is at least 80%, at least 85%, at least 90%, at least 95%, at least 96%, at least 97%, at least 98%, at least 99%, or 100% complementary to all or part of the endogenous GA20 oxidase_3 or GA20 oxidase_5 gene.
6. The modified corn plant, or plant part thereof, of embodiment 5, wherein the transcribable DNA sequence is at least 80%, at least 85%, at least 90%, at least 95%, at least 96%, at least 97%, at least 98%, at least 99%, or 100% complementary to a RNA transcript sequence, or a portion thereof, encoded by the endogenous GA20 oxidase_3 or GA20 oxidase_5 gene.
7. The modified corn plant, or plant part thereof, of embodiment 5, wherein the transcribable DNA sequence is at least 80%, at least 85%, at least 90%, at least 95%, at least 96%, at least 97%, at least 98%, at least 99%, or 100% complementary to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 1-3, 5-7, 9, and 11-38.
8. The modified corn plant, or plant part thereof, of embodiment 5, wherein the transcribable DNA sequence is at least 80%, at least 85%, at least 90%, at least 95%, at least 96%, at least 97%, at least 98%, at least 99%, or 100% complementary to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 5-7 and 11-18.
9. The modified corn plant, or plant part thereof, of any one of embodiments 1-8, wherein the genome modification further deletes at least a portion of the transcription termination sequence of the endogenous GA20 oxidase_5 gene.
10. The modified corn plant, or plant part thereof, of any one of embodiments 1-9, wherein the genome modification comprises a deletion of one or both of the transcription termination sequences of the endogenous GA20 oxidase_5 and SAMT genes.
11. The modified corn plant, or plant part thereof, of any one of embodiments 1-10, wherein the genome modification comprises a deletion of at least 25, at least 30, at least 40, at least 50, at least 75, at least 100, at least 150, at least 200, at least 300, at least 400, at least 500, at least 750, at least 1000 consecutive nucleotides of the intergenic region between the endogenous GA20 oxidase_5 and SAMT genes.
12. The modified corn plant, or plant part thereof, of any one of embodiments 1-11, wherein the genome modification comprises a deletion of the entire intergenic region between the endogenous GA20 oxidase_5 and SAMT genes.
13. The modified corn plant, or plant part thereof, of any one of embodiments 1-12, wherein the genome modification comprises a deletion of one or more sequence elements selected from the group consisting of the 5′ UTR, 1^{st} exon, 1^{st} intron, 2^{nd} exon, 2^{nd} intron, 3^{rd} exon, 3′ UTR, and any portion of the foregoing, of the endogenous GA20 oxidase_5 gene.
14. The modified corn plant, or plant part thereof, of any one of embodiments 1-13, wherein the genome modification comprises a deletion of one or more sequence elements selected from the group consisting of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion of the foregoing, of the endogenous Zm.SAMT locus.
15. The modified corn plant, or plant part thereof, of any one of embodiments 1-14, wherein the mutant allele produces a RNA molecule comprising an antisense sequence that is at least 80%, at least 85%, at least 90%, at least 95%, at least 96%, at least 97%, at least 98%, at least 99%, or 100% complementary to a RNA transcript sequence, or a portion thereof, encoded by the endogenous GA20 oxidase_5 gene.
16. The modified corn plant, or plant part thereof, of any one of embodiments 1-15, wherein the RNA transcript sequence comprises a sequence that is at least 90%, at least 95%, at least 96%, at least 97%, at least 98%, at least 99%, or 100% identical to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 1-3, 5-7, 9, and 11-38.
17. The modified corn plant, or plant part thereof, of any one of embodiments 1-16, wherein the RNA transcript sequence comprises a sequence that is at least 90%, at least 95%, at least 96%, at least 97%, at least 98%, at least 99%, or 100% identical to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 5-7 and 11-18.
18. The modified corn plant, or plant part thereof, of any one of embodiments 1-17, wherein the antisense sequence of the RNA molecule is at least 80%, at least 85%, at least 90%, at least 95%, at least 96%, at least 97%, at least 98%, at least 99%, or 100% complementary to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 1-3, 5-7, 9, and 11-38.
19. The modified corn plant, or plant part thereof, of any one of embodiments 1-18, wherein the antisense sequence of the RNA molecule is at least 80%, at least 85%, at least 90%, at least 95%, at least 96%, at least 97%, at least 98%, at least 99%, or 100% complementary to at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, or at least 3000 consecutive nucleotides of one or more of SEQ ID NOs: 5-7 and 11-18.
20. The modified corn plant, or plant part thereof, of any one of embodiments 1-19, wherein the genome modification results in the production of an RNA molecule comprising an antisense sequence from a genomic segment of selected from the group consisting of an exon, a portion of an exon, an intron, a portion of an intron, a 5′ or 3′ untranslated region (UTR), a portion of an UTR, and any combination of the foregoing, of the endogenous GA20 oxidase_5 locus.
21. The modified corn plant, or plant part thereof, of any one of embodiments 1-20, wherein the antisense sequence can hybridize with an RNA transcript encoded by a wild-type allele of one or both of the endogenous GA20 oxidase_3 gene and the endogenous GA20 oxidase_5 gene.
22. The modified corn plant, or plant part thereof, of any one of embodiments 1-21, wherein the antisense sequence can hybridize with a sense RNA transcript encoded by an endogenous GA20 oxidase_5 gene.
23. The modified corn plant, or plant part thereof, of any one of embodiments 1-21, wherein the antisense sequence can hybridize with a sense RNA transcript encoded by the mutant allele of the endogenous GA20 oxidase_5 gene.
24. The modified corn plant, or plant part thereof, of embodiment 22 or 23, wherein the sense RNA transcript encoded by the mutant allele of the endogenous GA20 oxidase_5 gene is shortened or truncated relative to a wild-type allele of the endogenous GA20 oxidase_5 gene.
25. The modified corn plant, or plant part thereof, of any one of embodiments 21-25, wherein the hybridization can cause suppression of a wild-type or mutant allele of the endogenous GA20 oxidase_3 gene, a wild-type or mutant allele of the endogenous GA20 oxidase_5 gene, or a wild-type or mutant allele of both genes.
26. The modified corn plant, or plant part thereof, of any one of embodiments 1-25, wherein the genome modification comprises two or more, three or more, four or more, five or more, or six or more non-contiguous deletions.
27. A modified corn plant, or plant part thereof, comprising a mutant allele of the endogenous GA20 oxidase_5 locus, wherein the mutant allele comprises a genome modification which results in the transcription of an antisense strand of at least an exon, an intron, or an untranslated region (UTR) of the endogenous GA20 oxidase_5 gene, or any portion thereof
28. A modified corn plant, or plant part thereof, comprising a mutant allele of the endogenous GA20 oxidase_5 locus, wherein the mutant allele comprises the Zm.SAMT gene promoter, or a functional part thereof, operably linked to at least one transcribable antisense sequence of at least an exon, intron or untranslated region (UTR) of the endogenous GA20 oxidase_5 gene, or any portion thereof
29. A modified corn plant, or plant part thereof, comprising a mutant allele of the endogenous GA20 oxidase_5 locus, wherein the mutant allele comprises a sequence selected from the group consisting of SEQ ID NOs: 87-105.
30. A modified corn plant, or plant part thereof, comprising a mutant allele of the endogenous GA20 oxidase_5 locus, wherein the mutant allele comprises a first sequence and a second sequence; wherein the first sequence comprises one or more of the 5′ UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene; wherein the first sequence and the second sequence are contiguous or separated only by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.
31. A modified corn plant, or plant part thereof, comprising a mutant allele of the endogenous GA20 oxidase_5 locus, wherein the mutant allele comprises a genomic deletion relative to a wild type allele of the endogenous GA20 oxidase_5 locus, wherein the genomic deletion is flanked by a first sequence and a second sequence; wherein the first sequence comprises one or more of the 5′ UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene.
32. A modified corn plant, or plant part thereof, comprising a mutant allele of the endogenous GA20 oxidase_5 locus, wherein the mutant allele comprises a genomic sequence comprising a first sequence and a second sequence; wherein the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 11-18 and 59-66; wherein the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 18-38 and 39-59; and wherein the genomic sequence is at least 50, at least 75, at least 100, at least 150, at least 200, at least 300, at least 400, at least 500, at least 750, at least 1000, at least 1500, at least 2000, at least 2500, at least 3000, at least 3500, at least 4000, at least 4500, or at least 5000, at least 5500, at least 6000, at least 6500, at least 7000, at least 7500, or at least 8000 consecutive nucleotides in length, and/or fewer than 9000, fewer than 8500, fewer than 8000, fewer than 7500, fewer than 7000, fewer than 6500, fewer than 6000, fewer than 5500, fewer than 5000, fewer than 4500, fewer than 4000, fewer than 3500, fewer than 3000, fewer than 2500, fewer than 2000, fewer than 1500, fewer than 1000, fewer than 750, fewer than 500, fewer than 250, fewer than 200, fewer than 150, fewer than 100, or fewer than 50 consecutive nucleotides in length.
33. The modified corn plant, or plant part thereof, of any one of embodiments 30, 31 or 32, wherein the first sequence comprises one or more of SEQ ID NOs: 11-18 and 59-66, or any portion thereof, and wherein the second sequence comprises one or more of SEQ ID NOs: 18-38 and 39-59, or any portion thereof
34. The modified corn plant, or plant part thereof, of any one of embodiments 30, 31 or 32, wherein the first sequence comprises one or more of SEQ ID NOs: 9-18 and 59-66, or any portion thereof, and wherein the second sequence comprises one or more of SEQ ID NOs: 9, 10, 18-38 and 39-59, or any portion thereof
35. The modified corn plant, or plant part thereof, of any one of embodiments 30-34, wherein the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 9-18 and 59-66, and wherein the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 9, 10, 18-38 and 39-59.
36. The modified corn plant, or plant part thereof, of any one of embodiments 31-35, wherein the genomic deletion comprises a deletion of the intergenic region between the endogenous Zm.GA20 oxidase_5 and Zm.SAMT genes.
37. The modified corn plant, or plant part thereof, of any one of embodiments 31-36, wherein the genomic deletion has a length of at least 250, at least 500, at least 750, at least 1000, at least 1250, at least 1500, at least 2000, at least 3000, at least 4000, at least 5000, at least 6000, at least 7000, or at least 7500 nucleotides.
38. The modified corn plant, or plant part thereof, of any one of embodiments 31-37, wherein the genomic deletion has a length of at most 1000, at most 1250, at most 1500, at most 2000, at most 3000, at most 4000, at most 5000, at most 6000, at most 7000, or at most 7500 nucleotides.
39. The modified corn plant, or plant part thereof, of any one of embodiments 31-38, wherein the genomic deletion corresponds to a deletion of one or more genomic regions comprising a sequence selected from the group consisting of SEQ ID NOs. 11-66.
40. The modified corn plant, or plant part thereof, of any one of embodiments 31-39, wherein the genome deletion results in the production of an RNA transcript comprising an antisense sequence from a genomic segment of the endogenous GA20 oxidase_5 locus selected from the group consisting of an exon, portion of an exon, an intron, portion of an intron, an untranslated region (UTR), portion of an UTR, and any combination of the foregoing.
41. The modified corn plant, or plant part thereof, of any one of embodiments 27-40, wherein the mutant allele can suppress the expression of a wild-type allele of the endogenous GA20 oxidase_3 locus, a wild-type allele of the endogenous GA20 oxidase_5 locus, or both.
42. The modified corn plant, or plant part thereof, of any of embodiments 1 to 41, wherein the corn plant is homozygous for the mutant allele at the endogenous GA20 oxidase_5 locus.
43. The modified corn plant, or plant part thereof, of any of embodiments 1 to 41, wherein the corn plant is heterozygous for the mutant allele at the endogenous GA20 oxidase_5 locus.
44. The modified corn plant, or plant part thereof, of any one of embodiments 1 to 43, wherein the modified corn plant has a shorter plant height and/or improved lodging resistance relative to an unmodified control plant.
45. The modified corn plant, or plant part thereof, of any one of embodiments 1 to 44, wherein the modified corn plant exhibits an at least 2.5%, at least 5%, at least 7.5%, at least 10%, at least 15%, at least 20%, at least 25%, at least 30%, at least 35%, or at least 40% reduction in plant height at maturity relative to an unmodified control plant.
46. The modified corn plant, or plant part thereof, of any one of embodiments 1-45, wherein the plant height reduction is between 5% and 40%, between 10% and 40%, between 15% and 40%, between 20% and 40%, between 30% and 40%, between 10% and 30%, between 15% and 30%, between 20% and 30%, between 5% and 30%, between 7.5% and 25%, between 10 and 20%, 5% and 7.5%, between 7.5% and 10%, between 10 and 15%, or between 15% to 20%.
47. The modified corn plant, or plant part thereof, of any one of embodiments 1 to 46, wherein the stalk or stem diameter of the modified corn plant at one or more stem internodes is at least 5%, at least 10%, at least 15%, at least 20%, at least 25%, at least 30%, at least 35%, or at least 40% greater than the stalk or stem diameter at the same one or more internodes of an unmodified control plant.
48. The modified corn plant, or plant part thereof, of any one of embodiments 1 to 47, wherein the stalk or stem diameter of the modified corn plant at one or more of the first, second, third, and/or fourth internode below the ear is at least 5%, at least 10%, at least 15%, at least 20%, at least 25%, at least 30%, at least 35%, or at least 40% greater than the same internode of an unmodified control plant.
49. The modified corn plant, or plant part thereof, of any one of embodiments 1 to 48, wherein the level of one or more active GAs in at least one internode tissue of the stem or stalk of the modified corn plant is at least 5%, at least 10%, at least 15%, at least 20%, at least 25%, at least 30%, at least 35%, or at least 40% lower than the same internode tissue of an unmodified control plant.
50. The modified corn plant, or plant part thereof, of any one of embodiments 1 to 49, wherein the level of one or more active GAs in at least one internode tissue of the stem or stalk of the modified corn plant is lower than the same internode tissue of an unmodified control plant.
51. The modified corn plant, or plant part thereof, of any one of embodiments 1 to 50, wherein the modified corn plant does not have any significant off-types in at least one female organ or ear.
52. The modified corn plant, or plant part thereof, of any one of embodiments 1 to 51, wherein the modified corn plant exhibits essentially no reproductive abnormality.
53. A method for producing a modified corn plant comprising a mutant allele of the endogenous GA20 oxidase_5 locus, the method comprising:
   a. generating two double-stranded breaks (DSB) in or near the endogenous GA20 oxidase_5 locus in a corn cell using a targeted editing technique;
   b. developing or regenerating from the corn cell a corn plant, or plant part thereof, comprising a mutant allele of the endogenous GA20 oxidase_5 locus.
54. A method for producing a modified corn plant comprising a mutant allele of the endogenous GA20 oxidase_5 locus, the method comprising:
   a. generating a first and a second double-stranded breaks (DSB) in a corn cell using a targeted editing technique, wherein the first DSB is in a region selected from the group consisting of 5′ UTR, 1^{st} exon, 1^{st} intron, 2^{nd} exon, 2^{nd} intron, 3^{rd} exon, 3′ UTR, and any portion of the foregoing, of the endogenous GA20 oxidase_5 locus, and the intergenic region between the endogenous Zm.GA20 oxidase_5 gene and the endogenous Zm.SAMT gene; wherein the second DSB is in a region selected from the group consisting of 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion of the foregoing, of the endogenous Zm.SAMT locus, and the intergenic region between the endogenous Zm.GA20 oxidase_5 gene and the endogenous Zm.SAMT gene;
   b. developing or regenerating from the corn cell a corn plant, or plant part thereof, comprising a genomic deletion, wherein the genomic deletion is flanked by the first DSB and the second DSB.
55. The method of embodiment 53 or 54, wherein the mutant allele comprises a genome modification deleting or disrupting the transcription termination sequence of the endogenous Zm.SAMT locus, and/or deleting at least a portion of the intergenic region between the endogenous Zm.GA20 oxidase_5 and Zm.SAMT genes.
56. The method of embodiment 53 or 54, wherein the targeted editing technique comprises the use of at least one site-specific nuclease.
57. The method of embodiment 56, wherein the at least one site-specific nuclease is selected from the group consisting of a zinc-finger nuclease, a meganuclease, an RNA-guided nuclease, a TALE-nuclease, a recombinase, a transposase, and any combination thereof
58. The method of embodiment 56 or 57, wherein the at least one site-specific nuclease is a RNA-guided nuclease selected from the group consisting of a Cas9 nuclease or a variant thereof, and a Cpf1 nuclease or a variant thereof
59. The method of embodiment 53 or 54, wherein the method further comprises selecting a corn plant, or plant part thereof, comprising the genomic deletion.
60. A method for generating a corn plant comprising:
   (a) fertilizing at least one female corn plant with pollen from a male corn plant, where the at least one female corn plant and/or the male corn plant comprise(s) a mutant allele of the endogenous GA20 oxidase_5 locus, wherein the mutant allele comprises a genome modification comprising:
      (i) a deletion of at least a portion of the transcription termination sequence of the endogenous Zm.SAMT gene, and where the mutant allele produces a RNA molecule comprising an antisense sequence complementary to all or part of the sense strand of the endogenous GA20 oxidase_5 gene;
      (ii) (ii) a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene; or
      (iii) (iii) a deletion of at least a portion of one or more of the following: 5′ UTR, 1^{st} exon, 1^{st} intron, 2^{nd} exon, 2^{nd} intron, 3^{rd} exon, 3′ UTR, and any portion thereof, and the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion thereof, of the endogenous Zm.SAMT gene; and
   (b) obtaining at least one seed produced by said fertilizing of step (a).
61. The embodiment of claim 60, wherein said method further comprises (c) growing said at least one seed obtained in step (b) to generate at least one progeny corn plant comprising said mutant allele.
62. The embodiment of claim 60, wherein said at least one seed from step (b) is heterozygous for said mutant allele.
63. The embodiment of claim 60, wherein said at least one seed is homozygous for said mutant allele.
64. The method of any one of embodiments 60-63, wherein said female corn plant is homozygous for said mutant allele.
65. The method of any one of embodiments 60-63, wherein said female corn plant is heterozygous for said mutant allele.
66. The method of any one of embodiments 60-62, 6, or 65 wherein said male corn plant lacks said mutant allele.
67. The method of any one of embodiments 60-65, wherein said male corn plant is heterozygous for said mutant allele.
68. The method of any one of embodiments 60-66, wherein said male corn plant is homozygous for said mutant allele.
69. The method of any one of embodiments 61-68, wherein said at least one progeny corn plant has a shorter plant height and/or improved lodging resistance relative to an control plant lacking said mutant allele.
70. The method of any one of embodiments 61-68, wherein said at least one progeny corn plant has a shorter plant height and/or improved lodging resistance relative to said male corn plant.
71. The method of any one of embodiments 61-70, wherein said female corn plant is an inbred corn plant.
72. The method of any one of embodiments 61-70, wherein said female corn plant is a hybrid corn plant.
73. The method of any one of embodiments 61-70, wherein said male corn plant is an inbred corn plant.
74. The method of any one of embodiments 61-73, wherein said male corn plant is a hybrid corn plant.
75. The method of any one of embodiments 61-74, wherein said female corn plant is an elite corn plant line.
76. The method of any one of embodiments 61-75, wherein said male corn plant is an elite corn plant line.
77. The method of any one of embodiments 61-71, 73, 75, or 76, wherein said female corn plant is of a first inbred corn line or variety, and wherein said male corn plant is of a different, second inbred corn line or variety.
78. The method of any one of embodiments 61-77, wherein said female corn plant and said male corn plant are grown in a greenhouse or growth chamber.
79. The method of any one of embodiments 61-77, wherein said female corn plant and said male corn plant are grown outdoors.
80. The method of any one of embodiments 61-79, wherein said female corn plant has been detasseled.
81. The method of any one of embodiments 61-79, wherein said female corn plant is a cytoplasmically male sterile corn plant.
82. A modified corn plant part, corn cell, or corn tissue, comprising a mutant allele of the endogenous GA20 oxidase_5 locus, wherein the mutant allele comprises a genome modification comprising a deletion of at least a portion of the transcription termination sequence of the endogenous Zm.SAMT gene, and wherein the mutant allele produces a RNA molecule comprising an antisense sequence complementary to all or part of the sense strand of the endogenous GA20 oxidase_5 gene.
83. A modified corn plant part, corn cell, or corn tissue, comprising a mutant allele of the endogenous GA20 oxidase_5 locus, wherein the mutant allele comprises a genome modification comprising a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene.
84. A modified corn plant part, corn cell, or corn tissue, comprising a mutant allele of the endogenous GA20 oxidase_5 locus, wherein the mutant allele comprises a genome modification comprising a deletion of at least a portion of one or more of the following: 5′UTR, 1^{st} exon, 1^{st} intron, 2^{nd} exon, 2^{nd} intron, 3^{rd} exon, 3′ UTR, and any portion thereof, and the 5′UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion thereof, of the endogenous Zm.SAMT gene.
85. A modified corn plant part, corn cell, or corn tissue, comprising a mutant allele of the endogenous GA20 oxidase_5 locus, wherein the mutant allele comprises a genome modification which results in the transcription of an antisense strand of at least an exon, an intron, or an untranslated region (UTR) of the endogenous GA20 oxidase_5 gene, or any portion thereof
86. A modified corn plant part, corn cell, or corn tissue, comprising a mutant allele of the endogenous GA20 oxidase_5 locus, wherein the mutant allele comprises the Zm.SAMT gene promoter, or a functional part thereof, operably linked to at least one transcribable antisense sequence of at least an exon, intron or untranslated region (UTR) of the endogenous GA20 oxidase_5 gene, or any portion thereof
87. A modified corn plant part, corn cell, or corn tissue, comprising a mutant allele of the endogenous GA20 oxidase_5 locus, wherein the mutant allele comprises a sequence selected from the group consisting of SEQ ID NOs: 87-105.
88. A modified corn plant part, corn cell, or corn tissue, comprising a mutant allele of the endogenous GA20 oxidase_5 locus, wherein the mutant allele comprises a first sequence and a second sequence; wherein the first sequence comprises one or more of the 5′UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene; wherein the first sequence and the second sequence are contiguous or separated only by an intervening sequence of fewer than 555, fewer than 525, fewer than 500, fewer than 450, fewer than 400, fewer than 350, fewer than 300, fewer than 250, fewer than 200, fewer than 150, fewer than 100, fewer than 50, fewer than 25, fewer than 20, fewer than 15, fewer than 10, fewer than 5, or fewer than 2 nucleotides.
89. A modified corn plant part, corn cell, or corn tissue, comprising a mutant allele of the endogenous GA20 oxidase_5 locus, wherein the mutant allele comprises a genomic deletion relative to a wild type allele of the endogenous GA20 oxidase_5 locus, wherein the genomic deletion is flanked by a first sequence and a second sequence; wherein the first sequence comprises one or more of the 5′UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene.
90. A modified corn plant part, corn cell, or corn tissue, comprising a mutant allele of the endogenous GA20 oxidase_5 locus, wherein the mutant allele comprises a genomic sequence comprising a first sequence and a second sequence; wherein the first sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 11-18 and 59-66; wherein the second sequence comprises at least 15, at least 20, at least 25, at least 30, at least 40, at least 50, at least 75, at least 100, at least 150, at least 200, at least 300, at least 400, at least 500, at least 750, at least 1000, at least 1500, at least 2000, at least 2500, at least 3000, or at least 3500 consecutive nucleotides of one or more of SEQ ID NOs: 18-38 and 39-59; and wherein the genomic sequence is at least 50, at least 75, at least 100, at least 150, at least 200, at least 300, at least 400, at least 500, at least 750, at least 1000, at least 1500, at least 2000, at least 2500, at least 3000, at least 3500, at least 4000, at least 4500, or at least 5000, at least 5500, at least 6000, at least 6500, at least 7000, at least 7500, or at least 8000 consecutive nucleotides in length, and/or fewer than 9000, fewer than 8500, fewer than 8000, fewer than 7500, fewer than 7000, fewer than 6500, fewer than 6000, fewer than 5500, fewer than 5000, fewer than 4500, fewer than 4000, fewer than 3500, fewer than 3000, fewer than 2500, fewer than 2000, fewer than 1500, fewer than 1000, fewer than 750, fewer than 500, fewer than 250, fewer than 200, fewer than 150, fewer than 100, or fewer than 50 consecutive nucleotides in length.

Having described the present disclosure in detail, it will be apparent that modifications, variations, and equivalent aspects are possible without departing from the spirit and scope of the present disclosure as described herein and in the appended claims. Furthermore, it should be appreciated that all examples in the present disclosure are provided as non-limiting examples.

### EXAMPLES

### Example 1. Constructs for Creation of Dominant Negative Deletion Mutant Alleles

The endogenous Zm.GA20ox5 gene is separated from an endogenous Zm.SAMT gene in the maize genome by an intergenic region of about 550 bp, or by 1170 bp if measured between stop codons, with the Zm.SAMT gene positioned downstream and oriented in the opposite orientation relative to the Zm.GA20ox5 gene. The sequence of the genomic locus or region encompassing the Zm.GA20ox5 and Zm.SAMT genes is provided in SEQ ID NOs. 9 and 10. SEQ ID NO. 9 represents a sequence of the GA20ox5-SAMT genomic locus corresponding to the sense strand of the Zm.GA20ox5 gene and encompassing both Zm.GA20ox5 and Zm.SAMT genes (the “GA20ox5_SAMT genomic sequence” in Table 2). SEQ ID NO. 10 represents a sequence of the GA20ox5-SAMT genomic locus corresponding to the sense strand of the Zm.SAMT gene (i.e., the antisense strand of the Zm.GA20ox5 gene) and encompassing both Zm.GA20ox5 and Zm.SAMT genes (the “SAMT_GA20ox5 genomic sequence” in Table 2). The elements or regions of the genomic sequences encompassing both Zm.GA20ox5 and Zm.SAMT genes are annotated in Table 2 below by reference to the nucleotide coordinates of those elements or regions in each of SEQ ID NOs. 9 and 10. As proposed herein, if a genomic region between the neighboring Zm.GA20ox5 and Zm.SAMT genes (including possibly all or part of those genes) were deleted, then the endogenous Zm.SAMT gene promoter may drive expression of an antisense RNA transcript through all or part of the Zm.GA20ox5 gene that can hybridize to a separate RNA transcript expressed form one or both of the copies or alleles of the Zm.GA20ox5 and/or Zm.GA20ox3 gene(s). Since the Zm.GA20ox3 and Zm.GA20ox5 genes share a high level of nucleotide sequence similarity in their respective exon coding regions, the antisense RNA transcript expressed from the oppositely oriented Zm.SAMT gene promoter may hybridize to transcripts of both GA20 oxidase genes and cause the suppression or silencing of one or both of the Zm.GA20ox3 and/or Zm.GA20ox5 gene(s). Thus, a mutant allele having a deletion between the Zm.GA20ox5 and Zm.SAMT genes may behave as a dominant or semi-dominant negative mutation or allele by causing suppression or silencing of one or both (wild-type and/or mutant) copies or alleles of the endogenous Zm.GA20ox5 gene, in addition to possible further suppression or silencing of one or both copies or alleles of the endogenous Zm.GA20ox3 gene.

TABLE 2
Annotation of genomic sequence elements of Zm.GA20ox5 and Zm.SAMT genomic region

| | | Location in the | | Location in the | |
|---|---|---|---|---|---|
| | | GA20ox5_SAMT | | SAMT_GA20ox5 | |
| Gene Name | | genomic sequence | | genomic sequence | |
| or Region | Element/Feature | (SEQ ID NO: 9) | SEQ ID NO | (SEQ ID NO: 10) | SEQ ID NO |
| GA20ox5 | Promoter and | 1 . . . 398 | 11 | 8670 . . . 9067 | 66 |
| | 5′ UTR | | | | |
| GA20ox5 | Exon 1 | 399 . . . 1189 | 12 | 7879 . . . 8669 | 65 |
| GA20ox5 | Intron 1 | 1190 . . . 1304 | 13 | 7764 . . . 7878 | 64 |
| GA20ox5 | Exon 2 | 1305 . . . 1629 | 14 | 7439 . . . 7763 | 63 |
| GA20ox5 | Intron 2 | 1630 . . . 2595 | 15 | 6473 . . . 7438 | 62 |
| GA20ox5 | Exon 3 | 2596 . . . 2871 | 16 | 6197 . . . 6472 | 61 |
| GA20ox5 | 3′ UTR | 2872 . . . 3180 | 17 | 5888 . . . 6196 | 60 |
| Intergenic Region | | 3181 . . . 3736 | 18 | 5332 . . . 5887 | 59 |
| SAMT | 3′ UTR | 3737 . . . 4141 | 19 | 4927 . . . 5331 | 58 |
| SAMT | Exon 8 | 4042 . . . 4258 | 20 | 4810 . . . 5026 | 57 |
| SAMT | Intron 8 | 4259 . . . 4512 | 21 | 4556 . . . 4809 | 56 |
| SAMT | Exon 7 | 4513 . . . 4707 | 22 | 4361 . . . 4555 | 55 |
| SAMT | Intron 7 | 4708 . . . 4989 | 23 | 4079 . . . 4360 | 54 |
| SAMT | Exon 6 | 4990 . . . 5262 | 24 | 3806 . . . 4078 | 53 |
| SAMT | Intron 6 | 5263 . . . 5348 | 25 | 3720 . . . 3805 | 52 |
| SAMT | Exon 5 | 5349 . . . 5523 | 26 | 3545 . . . 3719 | 51 |
| SAMT | Intron 5 | 5524 . . . 6037 | 27 | 3031 . . . 3544 | 50 |
| SAMT | Exon 4 | 6038 . . . 6148 | 28 | 2920 . . . 3030 | 49 |
| SAMT | Intron 4 | 6129 . . . 6239 | 29 | 2829 . . . 2939 | 48 |
| SAMT | Exon 3 | 6240 . . . 6510 | 30 | 2558 . . . 2828 | 47 |
| SAMT | Intron 3 | 6511 . . . 6894 | 31 | 2174 . . . 2557 | 46 |
| SAMT | Exon 2 | 6895 . . . 7044 | 32 | 2024 . . . 2173 | 45 |
| SAMT | Intron 2 | 7045 . . . 7139 | 33 | 1929 . . . 2023 | 44 |
| SAMT | Exon 1 | 7140 . . . 8126 | 34 | 942 . . . 1928 | 43 |
| SAMT | 5′ UTR 2 | 8127 . . . 8268 | 35 | 800 . . . 941 | 42 |
| SAMT | Intron 1 | 8269 . . . 8771 | 36 | 297 . . . 799 | 41 |
| SAMT | 5′ UTR 1 | 8772 . . . 8942 | 37 | 126 . . . 296 | 40 |
| SAMT | Promoter | 8943 . . . 9067 | 38 | 1 . . . 125 | 39 |

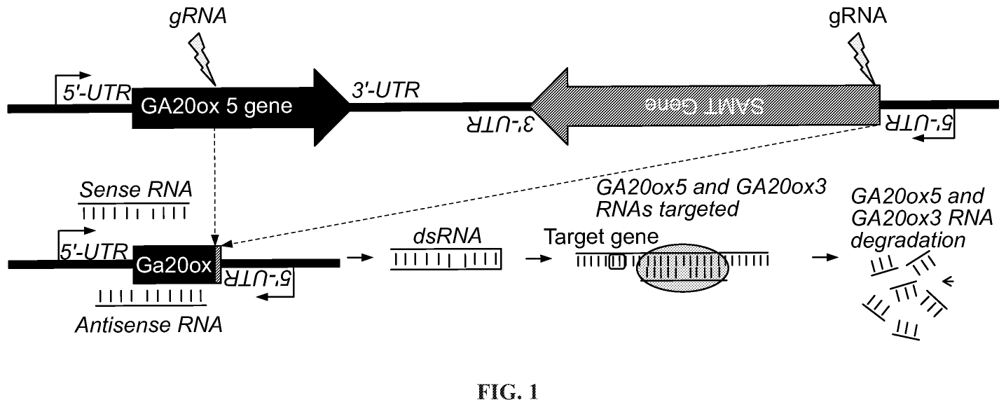

FIG. 1 illustrates the concept for creating an antisense RNA molecule that targets the Zm.GA20ox5 gene by deleting a genomic region between the Zm.GA20ox5 and its neighboring Zm.SAMT gene oriented in the opposite direction, through genome editing. The deletion can be generated using two or more guide RNAs that create double stranded breaks in the genome at the two ends of the intended deletion. The antisense RNA molecule generated from the oppositely oriented Zm.SAMT gene promoter can then hybridize to a sense Zm.GA20ox5 RNA transcript and trigger suppression or silencing of one or both copies or alleles (wild-type or mutant) of the endogenous Zm.GA20ox5 gene. FIG. 1 provides an embodiment where small RNAs may be generated through RNA interference. However, it is envisioned that suppression or silencing of the Zm.GA20ox5 gene may occur through other mechanisms as provided herein, alternatively or in addition to any RNAi or PTGS forms of suppression. Given that the Zm.GA20ox3 and Zm.GA20ox5 genes share a high level of nucleotide sequence similarity in their respective coding regions, the antisense RNA transcript may also hybridize to RNA transcripts of the Zm.GA20ox3 gene and cause the suppression or silencing of one or both of the Zm.GA20ox3 and/or Zm.GA20ox5 gene(s). Thus, a deletion between the Zm.GA20ox5 and Zm.SAMT genes may act as a dominant or semi-dominant negative mutation or allele for one or both of the Zm.GA20ox3 and/or Zm.GA20ox5 gene(s).

In the illustrative example provided in FIG. 1, a pair of guide RNAs are used including one guide RNA having a targeting or spacer sequence designed to target a site in the GA20ox5 gene, and another guide RNA having a targeting or spacer sequence designed to target a site in the Zm.SAMT gene. The size of the deletion and the location of the two breakpoints at the ends of the deletions may be determined by selecting which guide RNAs are used with a RNA-guided endonuclease to create the genome breaks. By creating a double strand break at both target sites, a deletion of the intervening region can be generated that will condense the genomic locus and bring the oppositely oriented Zm.SAMT gene promoter into closer proximity to the GA20ox5 gene, such that the Zm.SAMT gene promoter can drive the expression of an antisense RNA transcript that reads through at least a portion of the GA20ox5 gene. Even though a 3′ portion of the GA20ox5 gene may be deleted, the remaining 5′ portion of the GA20ox5 gene can be sufficient for an antisense RNA transcript or molecule to be generated under the control of the Zm.SAMT gene promoter that causes suppression or silencing of the Zm.GA20ox3 and/or GA20ox5 gene(s). Thus, the presence of a single copy or allele of the deletion mutant may act in a dominant or semi-dominant negative manner to cause a corn plant to have a short stature, lodging resistant phenotype.

Deletions in the Zm.GA20ox5/Zm.SAMT genomic region were generated using three different plasmid vector constructs for transformation. Each vector construct comprises a functional cassette for the expression of Cpf1 (or Cas12a), and further comprises one or two functional cassettes for the expression of guide RNAs, in addition to a selectable marker gene and plasmid maintenance elements. For the pMON419316 and pMON416796 constructs, the Cpf1 (or Cas12a) expression cassette comprises a maize ubiquitin promoter (SEQ ID NO: 67) operably linked to a sequence encoding a wild-type Lachnospiraceae bacterium Cpf1 RNA-guided endonuclease enzyme (SEQ ID NO: 68) fused to two nuclear localization signals (SEQ ID NOs: 70 and 71). The wild-type Cpf1 expression cassette further contains a synthetic sequence (atggcg) which provides a start codon. For the pMON419318 construct, the Cpf1 (or Cas12a) expression cassette comprises a maize ubiquitin promoter (SEQ ID NO: 67) operably linked to a sequence encoding a Lachnospiraceae bacterium G532R/K595R mutant Cpf1 RNA-guided endonuclease enzyme (SEQ ID NO: 69) fused to two nuclear localization signals (SEQ ID NOs: 72 and 73). See, e.g., Gao, L. et al., Nature Biotechnol. 35(8): 789-792 (2017), the entire contents and disclosure of which are incorporated herein by reference.

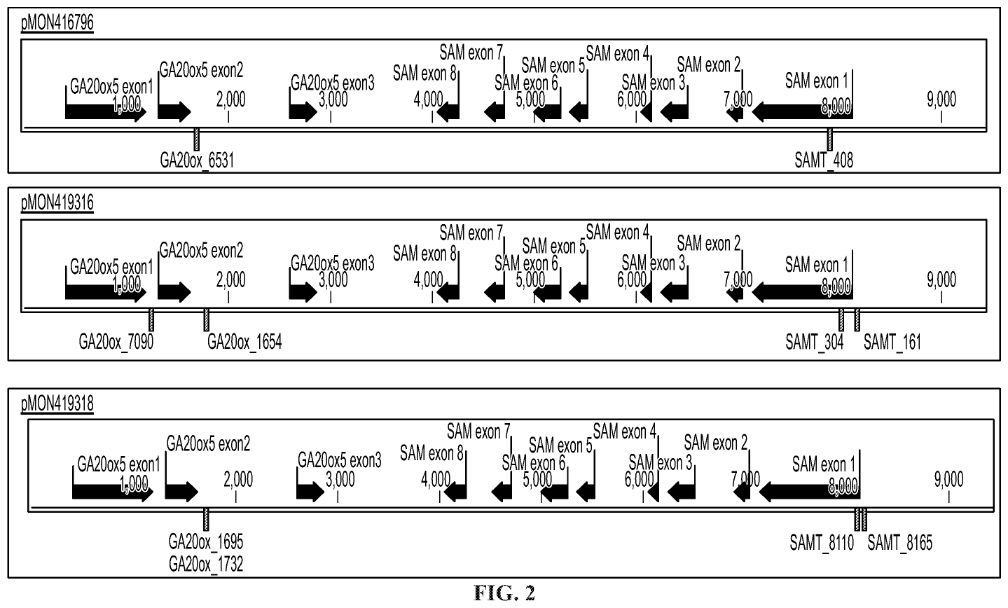

Table 3 below provides the target site, spacer and targeting/spacer sequence for each guide RNA encoded by the guide RNA cassette(s) in each vector construct. Each guide RNA unit within the guide RNA cassettes comprises a guide RNA scaffold sequence compatible with the LbCpf1 enzyme along with the unique spacer or targeting sequence complementary to its intended target site. For the pMON416796 construct, the guide RNA expression cassette comprises a maize RNA polymerase III (Pol3) promoter (SEQ ID NO: 74) operably linked to a sequence encoding two guide RNAs having targeting/spacer sequences encoded by the SP1b and SP1fDNA sequences in Table 3 below, with one guide RNA (SP1b) targeting a site in the first exon of the Zm.SAMT gene, and the other guide RNA (SP10 targeting a site in the first intron of the Zm.GA20ox5 gene (see also FIG. 2 (top panel) showing the placement of the two guide RNA target sites for SP1b and SP1f (SAMT_408 and GA20ox5_6531) relative to the genomic region encompassing the endogenous Zm.GA20ox5 and Zm.SAMT genes).

The pMON419316 construct has two guide RNA expression cassettes. One guide RNA expression cassette of the pMON419316 construct comprises a maize Pol3 promoter (SEQ ID NO: 74) operably linked to a sequence encoding two guide RNAs having targeting/spacer sequences encoded by the SP2f1 and SP2f2 DNA sequences in Table 3 below, with one guide RNA (SP2f1) targeting a site in the first exon of the Zm.GA20ox5 gene, and the other guide RNA (SP2f2) targeting a site in the second exon of the Zm.GA20ox5 gene. The other guide RNA expression cassette of the pMON419316 construct comprises a synthetic promoter operably linked to a sequence encoding two guide RNAs having targeting/spacer sequences encoded by the SP2b1 and SP2b2 DNA sequences in Table 3 below, with each guide RNA (SP2b1 and SP2b2) targeting different sites in the first exon of the Zm.SAMT gene. For the pMON419316 construct, see also the middle panel of FIG. 2 showing the placement of the four guide RNA target sites for SP2f1, SP2f1, SP2b1 and SP2b2 (GA20ox5_7090, GA20ox5_1654, SAMT_304 and SAMT_161) relative to the genomic region encompassing the endogenous Zm.GA20ox5 and Zm.SAMT genes.

The pMON419318 construct has two guide RNA expression cassettes. One guide RNA expression cassette of the pMON419318 construct comprises a maize Pol3 promoter (SEQ ID NO: 74) operably linked to a sequence encoding two guide RNAs having targeting/spacer sequences encoded by the SP3f1 and SP3f2 DNA sequences in Table 3 below, with each guide RNA (SP3f1 and SP3f2) targeting different sites in the second intron of the Zm.GA20ox5 gene. The other guide RNA expression cassette of the pMON419316 construct comprises a synthetic promoter operably linked to a sequence encoding two guide RNAs having targeting/spacer sequences encoded by the SP3b1 and SP3b2 DNA sequences in Table 3 below, with one guide RNA (SP3b1) targeting a site in the first exon of the Zm.SAMT gene, and another guide RNA (SP3b2) targeting a site in the 5′ UTR of the Zm.SAMT gene. For the pMON419318 construct, see also the lower panel of FIG. 2 showing the placement of the four guide RNA target sites for SP3f1, SP3f1, SP3b1 and SP3b2 (GA20ox5_1695_TYC, GA20ox5_1732_TYC, SAMT_8110_TYC and SAMT_8165_TYC) relative to the genomic region encompassing the endogenous Zm.GA20ox5 and Zm.SAMT genes.

TABLE 3
Transgenic constructs and their respective target sites and guide RNA
spacers

| | | | | |
|---|---|---|---|---|
| | guide | | | |
| | RNA | | | |
| Vector | Spacer | | | SEQ ID |
| Construct | ID | Target Site | Spacer Sequence | NO |

| | | | | |
|---|---|---|---|---|
| pMON416796 | SP1b | SAMT_408 | AGGACACCGACAACAATGATGCC | 75 |
| | SP1f | GA20ox5_6531 | GGTCCACTAGGATTCGGGAAATA | 76 |
| pMON419316 | SP2f1 | GA20ox5_7090 | GAGCCAATGGGGTAAGTAAGGTA | 77 |
| | SP2f2 | GA20ox5_1654 | GTTACCATGAAGGTGTCGCCGAT | 78 |
| | SP2b1 | SAMT_304 | GTCCAATAAGAAGCCGGTGGTGA | 79 |
| | SP2b2 | SAMT_161 | CACCTCGGCCAAATGCCATCAGT | 80 |
| pMON419318 | SP3f2 | GA20ox5_1695_TYC | GTTGAGCTCTCTCTGTGCTGTTA | 81 |
| | SP3f1 | GA20ox5_1732_TYC | CTAGGATTCGGGAAATAACAGCA | 82 |
| | SP3b1 | SAMT_8110_TYC | CCTCGGCCAAATGCCATCAGTGC | 83 |
| | SP3b2 | SAMT_8165_TYC | CGTGGTTTATCTCCACCAACAAC | 84 |

### Example 2. Characterization of Deletion Mutant Alleles of GA20Ox5 Gene

An inbred corn plant line was transformed via Agrobacterium-mediated transformation with a transformation vector having one of the constructs as described above in Example 1. The transformed plant tissue was grown to mature R0 plants. R0 plants having one or more unique genome edit(s) were selfed to produce R1 plants. To characterize the edits and recover plants with a deletion between the GA20Ox5 and SAMT genes, a PCR-based assay was performed using a pair of PCR primers flanking the intended deletion region. The same pair of primers (SEQ ID NOs: 85 and 86) were used for all three vectors in Table 3. If a deletion is present between the GA20Ox5 and SAMT genes, the PCR assay would result in an amplicon that could be sequenced. However, due to the large size of the intended deletion, the PCR assay would not produce a PCR product in the absence of a larger deletion. For each PCR assay, a 15 μL PCR reaction volume was used containing the Phusion PCR master mix from Thermo Fisher Scientific, 3 μL of genomic DNA template, and two PCR primers. After PCR amplification, a 3 μL PCR mixture was added to 21 μL of Tris-EDTA buffer and then analyzed on a ZAG instrument for the presence or absence of PCR products that indicate a GA20Ox5-SAMT deletion. The PCR products were sequenced to determine the junction sequence generated in each deletion around the GA20ox5-SAMT genomic locus (see Table 4).

R0 plants with a deletion between the GA20ox5 and SAMT genes were selected and selfed to produce R1 plants. The R1 plants were subject to a quantitative PCR assay to determine the zygosity of the GA20ox5-SAMT genomic locus (see Table 5). Each R1 plant was sequenced to determine all of the deletion edits around the GA20Ox5-SAMT genomic locus. Due to multiple gRNAs with a given construct, multiple deletions may occur on the same chromosome of a R0 plant and thus be present in a R1 plant, which may be homozygous or heterozygous for a mutant allele comprising the genomic deletion(s) (see Table 5). In Table 5, “homo” means homozygous for the mutant allele, and “hetero” means heterozygous for the mutant allele.

TABLE 4
Individual deletion junction sequences for edits made using the vectors in Table 3.

| SEQ | Deletion | | |
|---|---|---|---|
| ID | Junction | Junction Sequence | |
| NO. | Number | (with deletion size shown in the parentheses) | Junction Sequence Description |
| 87 | 1001 | GCGGCCGTCCATCTTTCCACCTCGGCCAAA-(-8)- | 8 nt deletion at SAMT_161 |
| | | GTGCCTGGCGAACATGTACCAGAGCACCAG | |
| 88 | 1002 | GGCCGTCCATCTTTCCACCTCGGCCAAATG-(-3)- | 3 nt deletion at SAMT_161 |
| | | TCAGTGCCTGGCGAACATGTACCAGAGCAC | |
| 89 | 1003 | GGCCGTCCATCTTTCCACCTCGGCCAAATG-(-6)- | 6 nt deletion at SAMT_161 |
| | | GTGCCTGGCGAACATGTACCAGAGCACCAG | |
| 90 | 1004 | GAGTGGCGCCCCGTCCGGCCCGTCCCGGGC-(-6357)- | 6357 nt deletion between |
| | | TTCTTATTGGACGAAATCTCCAGCGGGAAG | GA20ox5_1654 and SAMT_304 |
| 91 | 1005 | CCGGCCCGTCCCGGGCGCCATGGTCATCAA-(-6518)- | 6518 nt deletion between |
| | | GTGCCTGGCGAACATGTACCAGAGCACCAG | GA20ox5_1654 and SAMT_161 |
| 92 | 1006 | GTCCGGCCCGTCCCGGGCGCCATGGTCATC-(-6342)- | 6342 nt deletion between |
| | | GGCTTCTTATTGGACGAAATCTCCAGCGGG | GA20ox5_1654 and SAMT_304 |
| 93 | 1007 | GTCCGGCCCGTCCCGGGCGCCATGGTCATC-(-6348)- | 6348 nt deletion between |
| | | TTATTGGACGAAATCTCCAGCGGGAAGACA | GA20ox5_1654 and SAMT_304 |
| 94 | 1008 | CGTCCGGCCCGTCCCGGGCGCCATGGTCAT-(-6344)- | 6344 nt deletion between |
| | | GCTTCTTATTGGACGAAATCTCCAGCGGGA | GA20ox5_1654 and SAMT_304 |
| 95 | 1009 | CTGTGTGTATATTCAGTTGAGCTCTCTCTG-(-6478)- | 6478 nt deletion between |
| | | CACGGCTGGACCAACAGCCCCCCCAAAATC | GA20ox5_1695 and |
| | | | SAMT_8165 |
| 96 | 1010 | CTTGGCCGCTCTTGTCCTGTGTGTATATTC-(-6160)- | 6160 nt deletion between |
| | | GGTGTCCTCAAATTTCTCGGACCCTTCACC | GA20ox5_6531 and SAMT_408 |
| 97 | 1011 | TGTATATTCAGTTGAGCTCTCTCTGTGCTG-(-6133)- | 6133 nt deletion between |
| | | GTTGTCGGTGTCCTCAAATTTCTCGGACCC | GA20ox5_6531 and SAMT_408 |
| 98 | 1012 | TATATTCAGTTGAGCTCTCTCTGTGCTGTT-(-6130)- | 6130 nt deletion between |
| | | TGTTGTCGGTGTCCTCAAATTTCTCGGACC | GA20ox5_6531 and SAMT_408 |
| 99 | 1013 | ATATTCAGTTGAGCTCTCTCTGTGCTGTTA-(-6130)- | 6130 nt deletion between |
| | | GTTGTCGGTGTCCTCAAATTTCTCGGACCC | GA20ox5_6531 and SAMT_408 |
| 100 | 1014 | ATTCAGTTGAGCTCTCTCTGTGCTGTTATT-(-6131)- | 6131 nt deletion between |
| | | GTCGGTGTCCTCAAATTTCTCGGACCCTTC | GA20ox5_6531 and SAMT_408 |
| 101 | 1015 | CTCGGCCAGGATTTCGAGCCAATGGGGTAA-(-6759)- | 6759 nt deletion between |
| | | CTTCTTATTGGACGAAATCTCCAGCGGGAA | GA20ox5_7090 and SAMT_304 |
| 102 | 1016 | CGGCCAGGATTTCGAGCCAATGGGGTAAGT-(-6753)- | 6753 nt deletion between |
| | | CCGGCTTCTTATTGGACGAAATCTCCAGCG | GA20ox5_7090 and SAMT_304 |
| 103 | 1017 | TCGGCCAGGATTTCGAGCCAATGGGGTAAG-(-12)- | 12 nt deletion at GA20ox5_7090 |
| | | AAGGAGCGCCGGTTTACATTTACCGCACGT | |
| 104 | 1018 | TCGGCCAGGATTTCGAGCCAATGGGGTAAG-(-4)- | 4 nt deletion at GA20ox5_7090 |
| | | GTAGTAAGAAGGAGCGCCGGTTTACATTTA | |
| 105 | 1019 | GGACTACTTCGTCGGCACCCTCGGCCAGGA-(-39)- | 39 nt deletion at GA20ox5_7090 |
| | | GCCGGTTTACATTTACCGCACGTCGGCGTG | |

TABLE 5
Deletion edits and genotype of R0 ad R1 plants.

| | | | R1 zygosity | Deletion |
|---|---|---|---|---|
| | | | call for | Junction |
| | | | deletion | Number(s) |
| R0 Edit ID | R1 Plant ID | Editing Deletion Type | mutant | (Table 4) |
| E221089 | P43596818 | 6759 nt deletion between GA20ox5_7090 | Homozygous | 1015; 1003 |
| | | and SAMT_304; 6 nt deletion at T161 | | |
| E221089 | P43596820 | 6759 nt deletion between GA20ox5_7090 | Homozygous | 1015; 1003 |
| | | and SAMT_304; 6 nt deletion at T161 | | |
| E221089 | P43596823 | 6759 nt deletion between GA20ox5_7090 | Homozygous | 1015; 1003 |
| | | and SAMT_304; 6 nt deletion at T161 | | |
| E221089 | P43596801 | 6759 nt deletion between GA20ox5_7090 | Homozygous | 1015; 1003 |
| | | and SAMT_304; 6 nt deletion at T161 | | |
| E221089 | P43596831 | 6759 nt deletion between GA20ox5_7090 | Homozygous | 1015; 1003 |
| | | and SAMT_304; 6 nt deletion at T161 | | |
| E220938 | P43596469 | 6753 nt deletion between GA20ox5_7090 | Homozygous | 1016; 1001 |
| | | and SAMT_304; 8 nt deletion at T161 | | |
| E220938 | P43596438 | 6753 nt deletion between GA20ox5_7090 | Homozygous | 1016; 1001 |
| | | and SAMT_304; 8 nt deletion at T161 | | |
| E220938 | P43596489 | 6753 nt deletion between GA20ox5_7090 | Homozygous | 1016; 1001 |
| | | and SAMT_304; 8 nt deletion at T161 | | |
| E220242 | P95046375 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046377 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046392 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046378 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046370 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046369 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046368 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046395 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220242 | P95046396 | 6344 nt deletion between GA20ox5_1654 | Homozygous | 1008; |
| | | and SAMT_304; 12 nt deletion at | | 1017; 1002 |
| | | GA20ox5_7090; 3 nt deletion at SAMT_161 | | |
| E220698 | P43596662 | 6518 nt deletion between GA20ox5_1654 | Homozygous | 1005; 1018 |
| | | and SAMT_161; 4 nt deletion at T7090 | | |
| E220698 | P43596671 | 6518 nt deletion between GA20ox5_1654 | Heterozygous | 1005; 1018 |
| | | and SAMT_161; 4 nt deletion at T7090 | | |
| E220698 | P43596694 | 6518 nt deletion between GA20ox5_1654 | Homozygous | 1005; 1018 |
| | | and SAMT_161; 4 nt deletion at T7090 | | |
| E220698 | P43596679 | 6518 nt deletion between GA20ox5_1654 | Homozygous | 1005; 1018 |
| | | and SAMT_161; 4 nt deletion at T7090 | | |
| E220698 | P43596701 | 6518 nt deletion between GA20ox5_1654 | Heterozygous | 1005;1018 |
| | | and SAMT_161; 4 nt deletion at T7090 | | |
| E220698 | P43596654 | 6518 nt deletion between GA20ox5_1654 | Homozygous | 1005; 1018 |
| | | and SAMT_161; 4 nt deletion at T7090 | | |
| E220698 | P43596690 | 6518 nt deletion between GA20ox5_1654 | Heterozygous | 1005; 1018 |
| | | and SAMT_161; 4 nt deletion at T7090 | | |
| E220698 | P43596703 | 6518 nt deletion between GA20ox5_1654 | Homozygous | 1005;1018 |
| | | and SAMT_161; 4 nt deletion at T7090 | | |
| E220698 | P43596711 | 6518 nt deletion between GA20ox5_1654 | Homozygous | 1005; 1018 |
| | | and SAMT_161; 4 nt deletion at T7090 | | |
| E220055 | P95046321 | 6348 nt deletion between GA20ox5_1654 | Homozygous | 1007; 1019 |
| | | and SAMT_304; 39 nt deletion at T7090 | | |
| E220055 | P95046342 | 6348 nt deletion between GA20ox5_1654 | Homozygous | 1007; 1019 |
| | | and SAMT_304; 39 nt deletion at T7090 | | |
| E220055 | P95046314 | 6348 nt deletion between GA20ox5_1654 | Homozygous | 1007; 1019 |
| | | and SAMT_304; 39 nt deletion at T7090 | | |
| E220055 | P95046297 | 6348 nt deletion between GA20ox5_1654 | Homozygous | 1007; 1019 |
| | | and SAMT_304; 39 nt deletion at T7090 | | |
| E220228 | P43596770 | 6357 nt deletion between GA20ox5_1654 | Homozygous | 1004 |
| | | and SAMT_304 | | |
| E220141 | P43596991 | 6342 nt deletion between GA20ox5_1654 | Homozygous | 1006 |
| | | and SAMT_304 | | |
| E220141 | P43597019 | 6342 nt deletion between GA20ox5_1654 | Homozygous | 1006 |
| | | and SAMT_304 | | |
| E220141 | P43596954 | 6342 nt deletion between GA20ox5_1654 | Homozygous | 1006 |
| | | and SAMT_304 | | |
| E220141 | P43596970 | 6342 nt deletion between GA20ox5_1654 | Homozygous | 1006 |
| | | and SAMT_304 | | |
| E220141 | P43596980 | 6342 nt deletion between GA20ox5_1654 | Homozygous | 1006 |
| | | and SAMT_304 | | |
| E187994 | P43597077 | 6160 nt deletion between GA20ox5_6531 | Homozygous | 1010 |
| | | and SAMT_408 | | |
| E187994 | P43597052 | 6160 nt deletion between GA20ox5_6531 | Heterozygous | 1010 |
| | | and SAMT_408 | | |
| E187994 | P43597049 | 6160 nt deletion between GA20ox5_6531 | Heterozygous | 1010 |
| | | and SAMT_408 | | |
| E187994 | P43597037 | 6160 nt deletion between GA20ox5_6531 | Homozygous | 1010 |
| | | and SAMT_408 | | |
| E188579 | P43596586 | 6133 nt deletion between GA20ox5_6531 | Heterozygous | 1011 |
| | | and SAMT_408 | | |
| E188579 | P43596582 | 6133 nt deletion between GA20ox5_6531 | Heterozygous | 1011 |
| | | and SAMT_408 | | |
| E188579 | P43596603 | 6133 nt deletion between GA20ox5_6531 | Heterozygous | 1011 |
| | | and SAMT_408 | | |
| E188579 | P43596594 | 6133 nt deletion between GA20ox5_6531 | Homozygous | 1011 |
| | | and SAMT_408 | | |
| E188790 | P09617231 | 6130 nt deletion between GA20ox5_6531 | Homozygous | 1012 |
| | | and SAMT_408 | | |
| E188790 | P09617182 | 6130 nt deletion between GA20ox5_6531 | Heterozygous | 1012 |
| | | and SAMT_408 | | |
| E188790 | P09617144 | 6130 nt deletion between GA20ox5_6531 | Heterozygous | 1012 |
| | | and SAMT_408 | | |
| E188790 | P09617191 | 6130 nt deletion between GA20ox5_6531 | Heterozygous | 1012 |
| | | and SAMT_408 | | |
| E188790 | P09617225 | 6130 nt deletion between GA20ox5_6531 | Homozygous | 1012 |
| | | and SAMT_408 | | |
| E188790 | P09617216 | 6130 nt deletion between GA20ox5_6531 | Homozygous | 1012 |
| | | and SAMT_408 | | |
| E188790 | P09617192 | 6130 nt deletion between GA20ox5_6531 | Homozygous | 1012 |
| | | and SAMT_408 | | |
| E188790 | P09617208 | 6130 nt deletion between GA20ox5_6531 | Homozygous | 1012 |
| | | and SAMT_408 | | |
| E188569 | P43596926 | 6130 nt deletion between GA20ox5_6531 | Homozygous | 1013 |
| | | and SAMT_408 | | |
| E188569 | P43596908 | 6130 nt deletion between GA20ox5_6531 | Homozygous | 1013 |
| | | and SAMT_408 | | |
| E188569 | P43596931 | 6130 nt deletion between GA20ox5_6531 | Homozygous | 1013 |
| | | and SAMT_408 | | |
| E188569 | P43596895 | 6130 nt deletion between GA20ox5_6531 | Heterozygous | 1013 |
| | | and SAMT_408 | | |
| E188569 | P43596896 | 6130 nt deletion between GA20ox5_6531 | Heterozygous | 1013 |
| | | and SAMT_408 | | |
| E188569 | P43596911 | 6130 nt deletion between GA20ox5_6531 | Heterozygous | 1013 |
| | | and SAMT_408 | | |
| E189115 | P43596944 | 6131 nt deletion between GA20ox5_6531 | Homozygous | 1014 |
| | | and SAMT_408 | | |
| E180294 | P43596566 | 6478 nt deletion between GA20ox5_1695 | Homozygous | 1009 |
| | | and SAMT_8165 | | |
| E180294 | P43596550 | 6478 nt deletion between GA20ox5_1695 | Homozygous | 1009 |
| | | and SAMT_8165 | | |
| E180294 | P43596542 | 6478 nt deletion between GA20ox5_1695 | Homozygous | 1009 |
| | | and SAMT_8165 | | |
| E180294 | P43596530 | 6478 nt deletion between GA20ox5_1695 | Homozygous | 1009 |
| | | and SAMT_8165 | | |
| E180294 | P43596524 | 6478 nt deletion between GA20ox5_1695 | Homozygous | 1009 |
| | | and SAMT_8165 | | |
| E180294 | P43596534 | 6478 nt deletion between GA20ox5_1695 | Homozygous | 1009 |
| | | and SAMT_8165 | | |
| E180294 | P43596558 | 6478 nt deletion between GA20ox5_1695 | Homozygous | 1009 |
| | | and SAMT_8165 | | |
| E180294 | P43596538 | 6478 nt deletion between GA20ox5_1695 | Homozygous | 1009 |
| | | and SAMT_8165 | | |

### Example 3. Reduced Plant Height of Corn Plants with Edited Allele

R1 corn plants homozygous or heterozygous for an edited allele of the GA20 oxidase 5 gene (as identified in Example 2) were grown to maturity to measure their plant heights along with wild type control plants. R1 seeds were planted in soil and grown to maturity in the greenhouse under day/night temperatures of 85°/70° and 16/8 hours of photoperiod using standard nutrient and light conditions for corn plant growth and development. Plant heights (PHT) of R1 plants were measured at R2 growth stage from the soil level to the base of the uppermost fully expanded leaf.

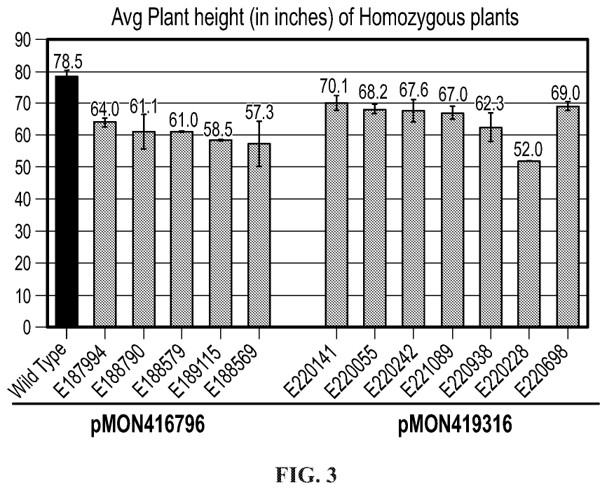

Table 6 provides the plant heights of individual R1 plants homozygous for deletion edits between the GA20ox5 and SAMT genes made using the pMON416796 or pMON419316 construct described in Example 1, along with wild type (WT) control plants. Average plant heights for WT and each homozygous deletion edit are also provided in Table 6 (see also FIG. 3 showing the average plant heights with error bars). These plant heights demonstrate that plants homozygous for an edited GA20 oxidase 5 allele comprising a deletion between the GA20ox5 and SAMT genes have significantly reduced plant heights averaging between 57.3 inches and 70.1 inches for plants having the edited alleles, versus an average plant height of 78.5 inches for the WT control.

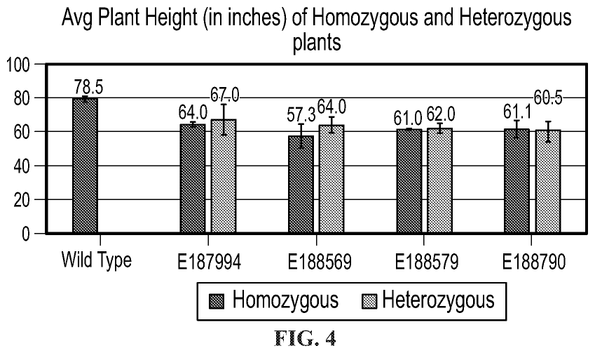

Table 7 provides the plant heights of individual R1 plants homozygous or heterozygous for deletion edits between the GA20ox5 and SAMT genes made using the pMON416796 construct described in Example 1, along with wild type (WT) control plants (see also FIG. 4 showing average plant heights with error bars). The data in Table 7 overlaps with Table 6 since R1 plants homozygous for the deletion edits made using the pMON416796 construct and the wild type control plants are the same as in Table 6. These plant heights demonstrate that plants heterozygous or homozygous for the edited GA20 oxidase 5 alleles comprising a deletion between the GA20ox5 and SAMT genes and made using the pMON416796 construct have significantly reduced plant heights averaging between 57.3 inches and 64 inches for plants homozygous these edited alleles, and between 60.5 inches and 67 inches for plants heterozygous for these edited alleles, relative to an average plant height of 78.5 inches for the WT control plants. The reductions in plant height are similar between plants homozygous and heterozygous for the deletion edit alleles, but plant heights overall for plants comprising the deletion edit alleles regardless of zygosity are significantly lower than those of wild type control plants.

The plant height data described in this example demonstrate that deletion of the region between GA20ox5 and SAMT genes leads to reduced plant heights as compared to wild type control plants, for plants homozygous or heterozygous for the edited deletion alleles, suggesting that these deletion alleles of the GA20 oxidase 5 gene act in a dominant or semi-dominant manner to produce a reduced plant height phenotype (i.e., semi-dwarf or short stature corn plants), especially since edited loss-of-function alleles of the GA20 oxidase 3 or GA20 oxidase 5 genes alone without an antisense or inversion sequence have been shown to not produce short stature corn plants. See, e.g., Published PCT Application Nos. WO/2019/161149, WO/2019/161147 and WO/2019/161144, the entire contents and disclosures of which are incorporated herein by reference. Further plant height measurements will be made in subsequent generations to confirm the shorter plant height phenotype.

TABLE 6
Plant Heights of homozygous R1 plants
using pMON416796 and pMON419316.

| Editing | | | Plant height |
|---|---|---|---|
| Construct ID | Edit ID | R1 Plant ID | (inches) |
| pMON416796 | E187994 | P43597037 | 65 |
| pMON416796 | E187994 | P43597077 | 63 |
| | E187994 | | 64.0 |
| | Average | | |
| pMON416796 | E188569 | P43596931 | 65 |
| pMON416796 | E188569 | P43596908 | 55.75 |
| pMON416796 | E188569 | P43596926 | 51 |
| | E188569 | | 57.3 |
| | Average | | |
| pMON416796 | E188579 | P43596594 | 61 |
| pMON416796 | E188790 | P09617225 | 70 |
| pMON416796 | E188790 | P09617231 | 60.75 |
| pMON416796 | E188790 | P09617208 | 60.25 |
| pMON416796 | E188790 | P09617216 | 59.5 |
| pMON416796 | E188790 | P09617192 | 55 |
| | E188790 | | 61.1 |
| | Average | | |
| pMON416796 | E189115 | P43596944 | 58.5 |
| pMON419316 | E220055 | P95046314 | 69.5 |
| pMON419316 | E220055 | P95046342 | 69 |
| pMON419316 | E220055 | P95046321 | 68 |
| pMON419316 | E220055 | P95046297 | 66.25 |
| | E220055 | | 68.2 |
| | Average | | |
| pMON419316 | E220141 | P43596991 | 72 |
| pMON419316 | E220141 | P43596954 | 72 |
| pMON419316 | E220141 | P43596970 | 71.5 |
| pMON419316 | E220141 | P43596980 | 68 |
| pMON419316 | E220141 | P43597019 | 67 |
| | E220141 | | 70.1 |
| | Average | | |
| pMON419316 | E220228 | P43596770 | 52 |
| pMON419316 | E220242 | P95046370 | 74 |
| pMON419316 | E220242 | P95046369 | 71.5 |
| pMON419316 | E220242 | P95046392 | 69.5 |
| pMON419316 | E220242 | P95046395 | 69 |
| pMON419316 | E220242 | P95046378 | 69 |
| pMON419316 | E220242 | P95046368 | 66 |
| pMON419316 | E220242 | P95046396 | 65 |
| pMON419316 | E220242 | P95046377 | 64.75 |
| pMON419316 | E220242 | P95046399 | 64 |
| pMON419316 | E220242 | P95046375 | 63.5 |
| | E220242 | | 67.6 |
| | Average | | |
| pMON419316 | E220698 | P43596694 | 70 |
| pMON419316 | E220698 | P43596662 | 68 |
| | E220698 | | 69.0 |
| | Average | | |
| pMON419316 | E220938 | P43596438 | 68.5 |
| pMON419316 | E220938 | P43596469 | 60.5 |
| pMON419316 | E220938 | P43596489 | 58 |
| | E220938 | | 62.3 |
| | Average | | |
| pMON419316 | E221089 | P43596831 | 69 |
| pMON419316 | E221089 | P43596820 | 67 |
| pMON419316 | E221089 | P43596823 | 65 |
| | E221089 | | 67 |
| | Average | | |
| Wild type | WT1 | | 80 |
| Wild type | WT2 | | 79.5 |
| Wild type | WT3 | | 79 |
| Wild type | WT4 | | 79 |
| Wild type | WT5 | | 75 |
| | Wild type | | 78.5 |
| | Average | | |

TABLE 7
Plant Heights of homozygous and heterozygous
R1 plants using pMON416796.

| | | R1 zygosity for | Plant height |
|---|---|---|---|
| Event ID | R1 Plant ID | deletion mutant | (inches) |
| E187994 | P43597052 | Heterozygous | 60.5 |
| E187994 | P43597049 | Heterozygous | 73.5 |
| | E187994 | | 67.0 |
| | Heterozygous | | |
| | Average | | |
| E187994 | P43597037 | Homozygous | 65 |
| E187994 | P43597077 | Homozygous | 63 |
| | E187994 | | 64.0 |
| | Homozygous | | |
| | Average | | |
| E188569 | P43596895 | Heterozygous | 63.5 |
| E188569 | P43596911 | Heterozygous | 59.5 |
| E188569 | P43596896 | Heterozygous | 69 |
| | E188569 | | 64.0 |
| | Heterozygous | | |
| | Average | | |
| E188569 | P43596908 | Homozygous | 55.75 |
| E188569 | P43596926 | Homozygous | 51 |
| E188569 | P43596931 | Homozygous | 65 |
| | E188569 | | 57.3 |
| | Homozygous | | |
| | Average | | |
| E188579 | P43596582 | Heterozygous | 62 |
| E188579 | P43596603 | Heterozygous | 59 |
| E188579 | P43596586 | Heterozygous | 65 |
| | E188579 | | 62.0 |
| | Heterozygous | | |
| | Average | | |
| E188579 | P43596594 | Homozygous | 61 |
| E188790 | P09617182 | Heterozygous | 65.75 |
| E188790 | P09617238 | Heterozygous | 65 |
| E188790 | P09617144 | Heterozygous | 61 |
| E188790 | P09617191 | Heterozygous | 50.25 |
| | E188790 | | 60.5 |
| | Heterozygous | | |
| | Average | | |
| E188790 | P09617192 | Homozygous | 55 |
| E188790 | P09617208 | Homozygous | 60.25 |
| E188790 | P09617225 | Homozygous | 70 |
| E188790 | P09617231 | Homozygous | 60.75 |
| E188790 | P09617216 | Homozygous | 59.5 |
| | E188790 | | 61.1 |
| | Homozygous | | |
| | Average | | |
| Wild type | WT1 | | 80 |
| Wild type | WT2 | | 79.5 |
| Wild type | WT3 | | 79 |
| Wild type | WT4 | | 79 |
| Wild type | WT5 | | 75 |
| | Wild type | | 78.5 |
| | Average | | |

### Example 4. Collection of Samples from R2 Plants for Molecular Assays

For the E220141 and E221089 deletion edits from the pMON419316 construct, R1 plants homozygous for those deletion edits (P43596991 and P43596831, respectively) were selfed to produce homozygous inbred R2 plants. The R2 inbred plants containing one of the E220141 and E221089 edits, and wild type control plants of the same inbred line, were grown under standard conditions in the greenhouse and sampled at V2 growth stage for the molecular assays described below. The plants were cut just above the soil level and the entire above-ground portion of the plants were placed in 50 ml conical tubes and immediately frozen in liquid nitrogen. Each sample contained one or two sibling plants of the same genotype. The number of samples for each assay and genotype are provided in Table 8. The frozen samples were milled and used for the small RNA and GA hormone assays described in Examples 5 and 6 below.

TABLE 8
Description of samples for small RNA and GA hormones assays.

| | | Number of | Number of |
|---|---|---|---|
| Editing | Edit ID | samples for small | samples for GA |
| Construct ID | (R2 Inbreds) | RNA assay | hormone assay |
| Inbred Wild type | | 2 | |
| pMON419316 | E220141 | 2 | 7 |
| pMON419316 | E221089 | 1 | 10 |

### Example 5. Detection of Small RNAs in Plants Having an Edited Deletion Allele

To generate small RNA libraries for sequencing, Illumina's TruSeq small RNA Library Preparation Kit was used according to the manufacturer's protocol (Document #15004197v02) with a modification at the library purification step. Samples of each genotype for this small RNA assay experiment are identified in Example 4 above. After amplification of cDNA, individual libraries were gel purified using a 6% Novex TBE PAGE Gel for size separation. The gel was stained with 1×SYBR Gold for 20 minutes. The final library product was sequenced on Illumina's NextSeq platform with a minimum depth of 3 million reads per sample. After sequencing, reads were processed through the following steps: the sequencing adapters were trimmed; reads matching housekeeping noncoding RNAs were removed and libraries normalized to reads per million. Between 1 and 9 samples per genotype were assayed.

The mutated GA20 oxidase 5 (GA20ox5) gene containing the E220141 and E221089 deletion edits were predicted to produce antisense RNA transcripts spanning all or part of the coding sequence of the GA20ox5 gene under the control of the downstream native SAMT promoter in the reverse orientation that could hybridize to mRNA transcripts expressed from the wild type and/or mutant GA20 oxidase 5 alleles and/or the GA20 oxidase 3 gene or allele(s). Since antisense RNA sequences can trigger RNA interference (RNAi) and suppression of genes encoding identical or homologous RNA sequences, plants containing the deletion edits were assayed for the presence of small RNAs. Processing of the double stranded RNA would be expected to produce small RNAs of about 21, 22 or 24 nucleotides in length corresponding to the coding sequence of the GA20ox5 gene. In this experiment, the edited R2 plants, as well as wild type control plants, did not show a noticeable accumulation of small RNAs corresponding to the GA20ox5 gene in the 21, 22 or 24-nucleotide small RNA range, which was measured to be 0 or 1 read per million total sequencing reads (data not shown). These data indicate that the edited plants either do not produce small RNAs at the V2 growth stage sampled in this example or act through a different dominant negative mechanism. However, the pattern of expression of antisense RNA transcripts complementary to all or part of the coding sequence of the GA20 oxidase 5 gene is also dependent on the SAMT gene promoter, which may not drive expression (or expression at a sufficiently high level) at the V2 growth stage to produce a measurable effect on the levels of small RNAs. Without being bound by theory, it is possible that expression of antisense transcripts from an edited deletion allele of the endogenous GA20ox5 gene may be more robust at later stages of development and thus have a greater or more measurable effect on the level of small RNAs and RNAi suppression at those later stages.

Future experiments will also seek to determine whether the levels of GA20ox3 and/or GA20ox5 mRNA transcripts are reduced in plants homozygous or heterozygous for an edited GA20ox5 allele having a deletion between the GA20ox5 and SAMT genes, relative to controls.

### Example 6. Detection of GA Hormones in Plants Having an Edited Deletion Allele

Reduced expression of GA20 oxidase genes can alter the levels of GA hormones in corn plants, which can in turn affect plant height with lower levels of active GAs potentially reducing plant height. The levels of bioactive GA hormones and their precursors were measured in plants containing the edited GA20ox5 alleles. GA20 oxidase is active in the GA biosynthetic pathway and catalyzes the sequential oxidation of metabolic intermediates GA12 and GA53 into GA9 and GA20, respectively (the “early 13-hydroxylation pathway” and “non 13-hydroxylation pathway”). The primary bioactive forms of GA include GA1, GA3 and GA4, which are further downstream (3′) of GA20 oxidase activity and the GA9 and GA20 intermediates in the biosynthetic pathway. A reduction or suppression of the expression level and/or enzymatic function of GA20 oxidase genes, as may be expected with the GA20ox5 deletion edits, may result in reduction of downstream metabolites (GA20 and GA9) and accumulation of upstream precursors (GA53 and GA12).

For this experiment, samples were collected as provided in Example 4 above. Freshly frozen plant sample tissues were extracted and cleaned using Waters solid phase extraction MAX cartridge plate. GA hormones and 2 internal standards were analyzed using UPLC coupled with an ABSciex 5500 Mass Spectrometry with MRM method. The final GA hormone values were calculated based on the calibration curve with ABSciex software Multi-Quan. Each GA hormone calibration curve was in good linear fit, the R2 linear regression >0.99. The 8 technical controls per 96-well plate for each hormone were also included and evaluated in analytical process for meeting the standard criterion. GA levels were measured in terms of pmol/gram of sample tissue.

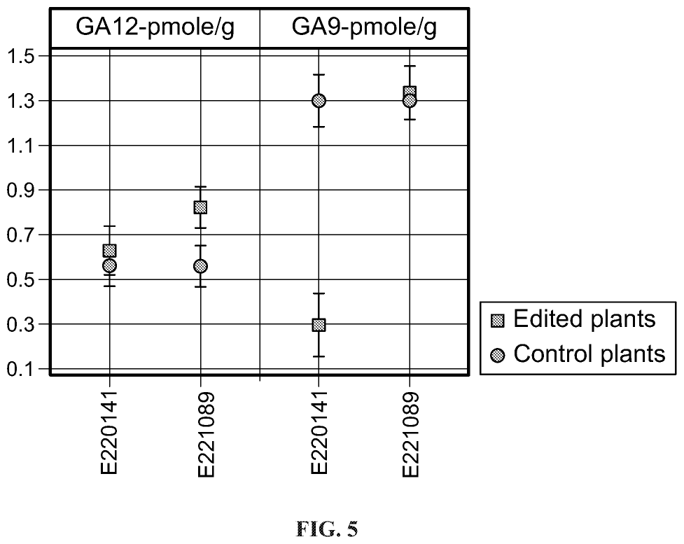

As shown in FIG. 5, the levels of GA12 were increased in inbred plants homozygous for the edited E221089 allele but were statistically neutral or unchanged in inbred plants homozygous for the edited E220141 allele, relative to wild type control plants. As further shown in FIG. 5, the levels of GA9 were decreased in inbred plants homozygous for the edited E220141 allele but neutral in inbred plants homozygous for the edited E221089 allele, relative to wild type control plants.

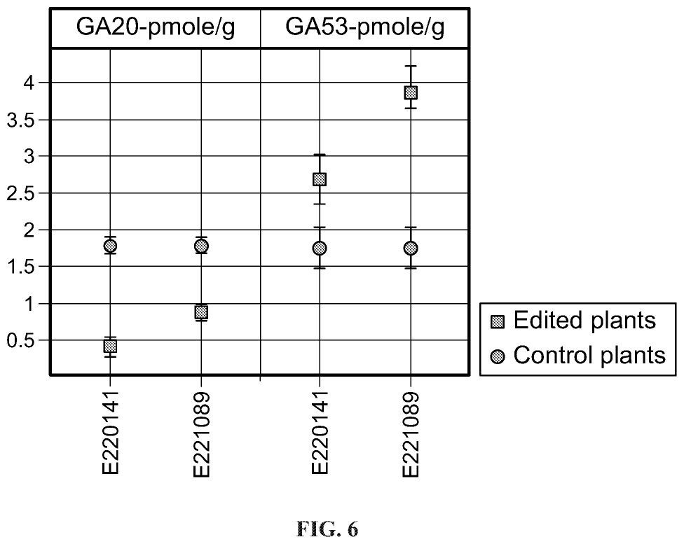

As shown in FIG. 6, the levels of GA20 were decreased in inbred plants homozygous for either of the edited alleles (E221089 or E220141), relative to wild type control plants. As further shown in FIG. 6, the levels of GA53 were increased in inbred plants homozygous for either of the edited alleles (E221089 or E220141), relative to wild type control plants.

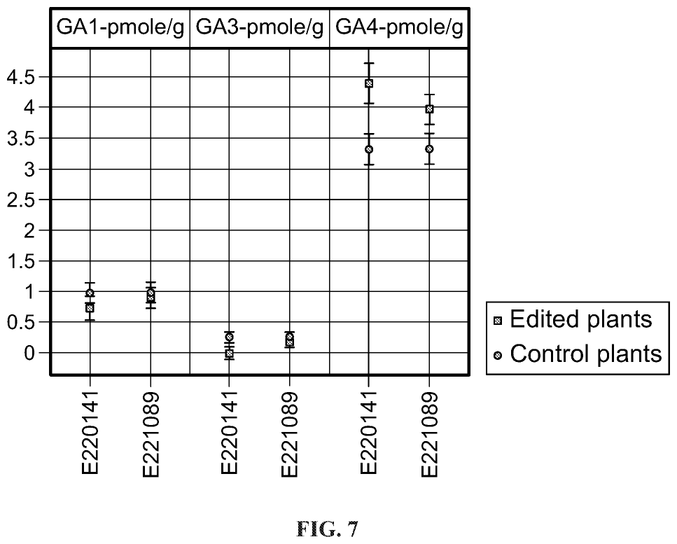

FIG. 7 provides the results for levels of active GAs (GA1, GA3 and GA4) measured in samples collected at V2 growth stage of the edited inbred plants relative to wild type controls. As shown in FIG. 7, the levels of these active GAs were generally not statistically changed in the inbred plants homozygous for the edited alleles (E221089 or E220141), except for an increase in GA4 in inbred plants homozygous for either of the edited alleles (E221089 or E220141).

These data support the theory that an antisense transcript may be expressed from the edited GA20 oxidase 5 gene, allele or locus having a deletion between the neighboring GA20 oxidase 5 and SAMT genes, that may reduce the expression level(s) of the GA20 oxidase 5 and/or GA20 oxidase 3 gene(s) and thus affect the levels of GA hormones in plants containing the edited alleles. The data in this experiment show increased accumulation of the GA12 and GA53 precursors upstream (5′) of GA20 oxidase activity and decreased levels of GA9 and GA20 products of GA20 oxidase activity in plants containing the edited GA20 oxidase 5 allele, although the levels of GA12 and GA9 were unchanged in the edited E220141 and E221089 inbred plants, respectively.

Although the levels of bioactive GAs were not shown to be reduced in this example, this may be due to the early V2 growth stage when the plant tissue samples were collected for this experiment. Indeed, the pattern of expression of an antisense RNA transcript complementary to all or part of the coding sequence of the GA20 oxidase 5 gene is dependent on the SAMT gene promoter, which may not drive expression (or expression at a sufficiently high level) at the early V2 growth stage to produce a measurable effect on the levels of active GAs. Without being bound by theory, it is possible that expression of antisense transcripts from the edited deletion alleles of the endogenous GA20ox5 gene under the control of the endogenous SAMT gene promoter may be more robust at later stages of development and thus have a greater or more measurable effect on the level(s) of active GAs at those later stages. The active GAs are also further downstream and not a direct product of GA20 oxidase enzyme activity. Future experiments will determine if lower active GA levels are observed at later stages of development in plants heterozygous or homozygous for an edited GA20 oxidase 5 locus comprising a deletion between the GA20ox5 and SAMT genes, which is supported by the altered levels of GA precursors observed in this example at the early V2 growth stage.

Having described the present disclosure in detail, it will be apparent that modifications, variations, and equivalent aspects are possible without departing from the spirit and scope of the present disclosure as described herein and in the appended claims. Furthermore, it should be appreciated that all examples in the present disclosure are provided as non-limiting examples.

What is claimed is:

1. A modified corn plant, or plant part thereof, comprising a mutant allele of the endogenous GA20 oxidase_5 locus, wherein the mutant allele comprises a genome modification comprising a deletion of at least a portion of the transcription termination sequence of the endogenous Zm.SAMT gene, and wherein the mutant allele produces a RNA molecule comprising an antisense sequence complementary to all or part of the sense strand of the endogenous GA20 oxidase_5 gene.

2. A modified corn plant, or plant part thereof, comprising a mutant allele of the endogenous GA20 oxidase_5 locus, wherein the mutant allele comprises a genome modification comprising a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene.

3. A modified corn plant, or plant part thereof, comprising a mutant allele of the endogenous GA20 oxidase_5 locus, wherein the mutant allele comprises a genome modification comprising a deletion of at least a portion of one or more of the following: 5′ UTR, 1^{st} exon, 1^{st} intron, 2^{nd} exon, 2^{nd} intron, 3^{rd} exon, 3′ UTR, and any portion thereof, and the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion thereof, of the endogenous Zm.SAMT gene.

4. The modified corn plant, or plant part thereof, of any one of claims 1-3, wherein the mutant allele comprises the endogenous Zm.SAMT gene promoter, or a portion thereof, operably linked to a transcribable DNA sequence encoding a RNA molecule that causes suppression of one or both of the endogenous GA20 oxidase_3 gene and the endogenous GA20 oxidase_5 gene.

5. The modified corn plant, or plant part thereof, of any one of claims 1-3, wherein the mutant allele comprises the endogenous Zm.SAMT gene promoter, or a portion thereof, operably linked to a transcribable DNA sequence encoding a RNA molecule comprising an antisense sequence that is at least 80% complementary to all or part of the endogenous GA20 oxidase_3 or GA20 oxidase_5 gene.

6. The modified corn plant, or plant part thereof, of claim 5, wherein the transcribable DNA sequence is at least 80% complementary to a RNA transcript sequence, or a portion thereof, encoded by the endogenous GA20 oxidase_3 or GA20 oxidase_5 gene.

7. The modified corn plant, or plant part thereof, of claim 5, wherein the transcribable DNA sequence is at least 80% complementary to at least 15 consecutive nucleotides of one or more of SEQ ID NOs: 1-3, 5-7, 9, and 11-38.

8. The modified corn plant, or plant part thereof, of claim 5, wherein the transcribable DNA sequence is at least 80% complementary to at least 15 consecutive nucleotides of one or more of SEQ ID NOs: 5-7 and 11-18.

9. The modified corn plant, or plant part thereof, of any one of claims 1-8, wherein the genome modification further deletes at least a portion of the transcription termination sequence of the endogenous GA20 oxidase_5 gene.

10. The modified corn plant, or plant part thereof, of any one of claim 1-9, wherein the genome modification comprises a deletion of one or both of the transcription termination sequences of the endogenous GA20 oxidase_5 and SAMT genes.

11. The modified corn plant, or plant part thereof, of any one of claims 1-10, wherein the genome modification comprises a deletion of at least 25 consecutive nucleotides of the intergenic region between the endogenous GA20 oxidase_5 and SAMT genes.

12. The modified corn plant, or plant part thereof, of any one of claims 1-11, wherein the genome modification comprises a deletion of the entire intergenic region between the endogenous GA20 oxidase_5 and SAMT genes.

13. The modified corn plant, or plant part thereof, of any one of claims 1-12, wherein the genome modification comprises a deletion of one or more sequence elements selected from the group consisting of the 5′ UTR, 1^{st} exon, 1^{st} intron, 2^{nd} exon, 2^{nd} intron, 3^{rd} exon, 3′ UTR, and any portion of the foregoing, of the endogenous GA20 oxidase_5 gene.

14. The modified corn plant, or plant part thereof, of any one of claims 1-13, wherein the genome modification comprises a deletion of one or more sequence elements selected from the group consisting of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion of the foregoing, of the endogenous Zm.SAMT locus.

15. The modified corn plant, or plant part thereof, of any one of claims 1-14, wherein the mutant allele produces a RNA molecule comprising an antisense sequence that is at least 80% complementary to a RNA transcript sequence, or a portion thereof, encoded by the endogenous GA20 oxidase_5 gene.

16. The modified corn plant, or plant part thereof, of any one of claims 1-15, wherein the RNA transcript sequence comprises a sequence that is at least 90% identical to at least 15 consecutive nucleotides of one or more of SEQ ID NOs: 1-3, 5-7, 9, and 11-38.

17. The modified corn plant, or plant part thereof, of any one of claims 1-16, wherein the RNA transcript sequence comprises a sequence that is at least 90 identical to at least 15 consecutive nucleotides of one or more of SEQ ID NOs: 5-7 and 11-18.

18. The modified corn plant, or plant part thereof, of any one of claims 1-17, wherein the antisense sequence of the RNA molecule is at least 80% complementary to at least 15 consecutive nucleotides of one or more of SEQ ID NOs: 1-3, 5-7, 9, and 11-38.

19. The modified corn plant, or plant part thereof, of any one of claims 1-18, wherein the antisense sequence of the RNA molecule is at least 80% complementary to at least 15 consecutive nucleotides of one or more of SEQ ID NOs: 5-7 and 11-18.

20. The modified corn plant, or plant part thereof, of any one of claims 1-19, wherein the genome modification results in the production of an RNA molecule comprising an antisense sequence from a genomic segment of selected from the group consisting of an exon, a portion of an exon, an intron, a portion of an intron, a 5′ or 3′ untranslated region (UTR), a portion of an UTR, and any combination of the foregoing, of the endogenous GA20 oxidase_5 locus.

21. The modified corn plant, or plant part thereof, of any one of claims 1-20, wherein the antisense sequence can hybridize with an RNA transcript encoded by a wild-type allele of one or both of the endogenous GA20 oxidase_3 gene and the endogenous GA20 oxidase_5 gene.

22. The modified corn plant, or plant part thereof, of any one of claims 1-21, wherein the antisense sequence can hybridize with a sense RNA transcript encoded by an endogenous GA20 oxidase_5 gene.

23. The modified corn plant, or plant part thereof, of any one of claims 1-21, wherein the antisense sequence can hybridize with a sense RNA transcript encoded by the mutant allele of the endogenous GA20 oxidase_5 gene.

24. The modified corn plant, or plant part thereof, of claim 22 or 23, wherein the sense RNA transcript encoded by the mutant allele of the endogenous GA20 oxidase_5 gene is shortened or truncated relative to a wild-type allele of the endogenous GA20 oxidase_5 gene.

25. The modified corn plant, or plant part thereof, of any one of claims 21-25, wherein the hybridization can cause suppression of a wild-type or mutant allele of the endogenous GA20 oxidase_3 gene, a wild-type or mutant allele of the endogenous GA20 oxidase_5 gene, or a wild-type or mutant allele of both genes.

26. The modified corn plant, or plant part thereof, of any one of claims 1-25, wherein the genome modification comprises two or more, three or more, four or more, five or more, or six or more non-contiguous deletions.

27. A modified corn plant, or plant part thereof, comprising a mutant allele of the endogenous GA20 oxidase_5 locus, wherein the mutant allele comprises a genome modification which results in the transcription of an antisense strand of at least an exon, an intron, or an untranslated region (UTR) of the endogenous GA20 oxidase_5 gene, or any portion thereof.

28. A modified corn plant, or plant part thereof, comprising a mutant allele of the endogenous GA20 oxidase_5 locus, wherein the mutant allele comprises the Zm.SAMT gene promoter, or a functional part thereof, operably linked to at least one transcribable antisense sequence of at least an exon, intron or untranslated region (UTR) of the endogenous GA20 oxidase_5 gene, or any portion thereof.

29. A modified corn plant, or plant part thereof, comprising a mutant allele of the endogenous GA20 oxidase_5 locus, wherein the mutant allele comprises a sequence selected from the group consisting of SEQ ID NOs: 87-105.

30. A modified corn plant, or plant part thereof, comprising a mutant allele of the endogenous GA20 oxidase_5 locus, wherein the mutant allele comprises a first sequence and a second sequence; wherein the first sequence comprises one or more of the 5′ UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene; wherein the first sequence and the second sequence are contiguous or separated only by an intervening sequence of fewer than 555 nucleotides.

31. A modified corn plant, or plant part thereof, comprising a mutant allele of the endogenous GA20 oxidase_5 locus, wherein the mutant allele comprises a genomic deletion relative to a wild type allele of the endogenous GA20 oxidase_5 locus, wherein the genomic deletion is flanked by a first sequence and a second sequence; wherein the first sequence comprises one or more of the 5′ UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene.

32. A modified corn plant, or plant part thereof, comprising a mutant allele of the endogenous GA20 oxidase_5 locus, wherein the mutant allele comprises a genomic sequence comprising a first sequence and a second sequence; wherein the first sequence comprises at least 15 consecutive nucleotides of one or more of SEQ ID NOs: 11-18 and 59-66; wherein the second sequence comprises at least 15 consecutive nucleotides of one or more of SEQ ID NOs: 18-38 and 39-59; and wherein the genomic sequence is at least 50 consecutive nucleotides in length, and/or fewer than 9000 consecutive nucleotides in length.

33. The modified corn plant, or plant part thereof, of claim 30, 31 or 32, wherein the first sequence comprises one or more of SEQ ID NOs: 11-18 and 59-66, or any portion thereof, and wherein the second sequence comprises one or more of SEQ ID NOs: 18-38 and 39-59, or any portion thereof.

34. The modified corn plant, or plant part thereof, of claim 30, 31 or 32, wherein the first sequence comprises one or more of SEQ ID NOs: 9-18 and 59-66, or any portion thereof, and wherein the second sequence comprises one or more of SEQ ID NOs: 9, 10, 18-38 and 39-59, or any portion thereof.

35. The modified corn plant, or plant part thereof, of any one of claims 30-34, wherein the first sequence comprises at least 15 consecutive nucleotides of one or more of SEQ ID NOs: 9-18 and 59-66, and wherein the second sequence comprises at least 15 consecutive nucleotides of one or more of SEQ ID NOs: 9, 10, 18-38 and 39-59.

36. The modified corn plant, or plant part thereof, of any one of claims 31-35, wherein the genomic deletion comprises a deletion of the intergenic region between the endogenous Zm.GA20 oxidase_5 and Zm.SAMT genes.

37. The modified corn plant, or plant part thereof, of any one of claims 31-36, wherein the genomic deletion has a length of at least 250 nucleotides.

38. The modified corn plant, or plant part thereof, of any one of claims 31-37, wherein the genomic deletion has a length of at most 7500 nucleotides.

39. The modified corn plant, or plant part thereof, of any one of claims 31-38, wherein the genomic deletion corresponds to a deletion of one or more genomic regions comprising a sequence selected from the group consisting of SEQ ID NOs. 11-66.

40. The modified corn plant, or plant part thereof, of any one of claims 31-39, wherein the genome deletion results in the production of an RNA transcript comprising an antisense sequence from a genomic segment of the endogenous GA20 oxidase_5 locus selected from the group consisting of an exon, portion of an exon, an intron, portion of an intron, an untranslated region (UTR), portion of an UTR, and any combination of the foregoing.

41. The modified corn plant, or plant part thereof, of any one of claims 27-40, wherein the mutant allele can suppress the expression of a wild-type allele of the endogenous GA20 oxidase_3 locus, a wild-type allele of the endogenous GA20 oxidase_5 locus, or both.

42. The modified corn plant, or plant part thereof, of any of claims 1 to 41, wherein the corn plant is homozygous for the mutant allele at the endogenous GA20 oxidase_5 locus.

43. The modified corn plant, or plant part thereof, of any of claims 1 to 41, wherein the corn plant is heterozygous for the mutant allele at the endogenous GA20 oxidase_5 locus.

44. The modified corn plant, or plant part thereof, of any one of claims 1 to 43, wherein the modified corn plant has a shorter plant height and/or improved lodging resistance relative to an unmodified control plant.

45. The modified corn plant, or plant part thereof, of any one of claims 1 to 44, wherein the modified corn plant exhibits an at least 2.5% reduction in plant height at maturity relative to an unmodified control plant.

46. The modified corn plant, or plant part thereof, of any one of claims 1-45, wherein the plant height reduction is between 5% and 40%.

47. The modified corn plant, or plant part thereof, of any one of claims 1 to 46, wherein the stalk or stem diameter of the modified corn plant at one or more stem internodes is at least 5% greater than the stalk or stem diameter at the same one or more internodes of an unmodified control plant.

48. The modified corn plant, or plant part thereof, of any one of claims 1 to 47, wherein the stalk or stem diameter of the modified corn plant at one or more of the first, second, third, and/or fourth internode below the ear is at least 5% greater than the same internode of an unmodified control plant.

49. The modified corn plant, or plant part thereof, of any one of claims 1 to 48, wherein the level of one or more active GAs in at least one internode tissue of the stem or stalk of the modified corn plant is at least 5% lower than the same internode tissue of an unmodified control plant.

50. The modified corn plant, or plant part thereof, of any one of claims 1 to 49, wherein the level of one or more active GAs in at least one internode tissue of the stem or stalk of the modified corn plant is lower than the same internode tissue of an unmodified control plant.

51. The modified corn plant, or plant part thereof, of any one of claims 1 to 50, wherein the modified corn plant does not have any significant off-types in at least one female organ or ear.

52. The modified corn plant, or plant part thereof, of any one of claims 1 to 51, wherein the modified corn plant exhibits essentially no reproductive abnormality.

53. A method for producing a modified corn plant comprising a mutant allele of the endogenous GA20 oxidase_5 locus, the method comprising:
a. generating two double-stranded breaks (DSB) in or near the endogenous GA20 oxidase_5 locus in a corn cell using a targeted editing technique;
b. developing or regenerating from the corn cell a corn plant, or plant part thereof, comprising a mutant allele of the endogenous GA20 oxidase_5 locus.

54. A method for producing a modified corn plant comprising a mutant allele of the endogenous GA20 oxidase_5 locus, the method comprising:
a. generating a first and a second double-stranded breaks (DSB) in a corn cell using a targeted editing technique, wherein the first DSB is in a region selected from the group consisting of 5′ UTR, 1^{st} exon, 1^{st} intron, 2^{nd} exon, 2^{nd} intron, 3^{rd} exon, 3′ UTR, and any portion of the foregoing, of the endogenous GA20 oxidase_5 locus, and the intergenic region between the endogenous Zm.GA20 oxidase_5 gene and the endogenous Zm.SAMT gene; wherein the second DSB is in a region selected from the group consisting of 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion of the foregoing, of the endogenous Zm.SAMT locus, and the intergenic region between the endogenous Zm.GA20 oxidase_5 gene and the endogenous Zm.SAMT gene;
b. developing or regenerating from the corn cell a corn plant, or plant part thereof, comprising a genomic deletion, wherein the genomic deletion is flanked by the first DSB and the second DSB.

55. The method of claim 53 or 54, wherein the mutant allele comprises a genome modification deleting or disrupting the transcription termination sequence of the endogenous Zm.SAMT locus, and/or deleting at least a portion of the intergenic region between the endogenous Zm.GA20 oxidase_5 and Zm.SAMT genes.

56. The method of claim 53 or 54, wherein the targeted editing technique comprises the use of at least one site-specific nuclease.

57. The method of claim 56, wherein the at least one site-specific nuclease is selected from the group consisting of a zinc-finger nuclease, a meganuclease, an RNA-guided nuclease, a TALE-nuclease, a recombinase, a transposase, and any combination thereof.

58. The method of claim 56 or 57, wherein the at least one site-specific nuclease is a RNA-guided nuclease selected from the group consisting of a Cas9 nuclease or a variant thereof, and a Cpf1 nuclease or a variant thereof.

59. The method of claim 53 or 54, wherein the method further comprises selecting a corn plant, or plant part thereof, comprising the genomic deletion.

60. A method for generating a corn plant comprising:
(a) fertilizing at least one female corn plant with pollen from a male corn plant, where the at least one female corn plant and/or the male corn plant comprise(s) a mutant allele of the endogenous GA20 oxidase_5 locus, wherein the mutant allele comprises a genome modification comprising:
(i) a deletion of at least a portion of the transcription termination sequence of the endogenous Zm.SAMT gene, and where the mutant allele produces a RNA molecule comprising an antisense sequence complementary to all or part of the sense strand of the endogenous GA20 oxidase_5 gene;
(ii) a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene; or
(iii) a deletion of at least a portion of one or more of the following: 5′ UTR, 1^{st} exon, 1^{st} intron, 2^{nd} exon, 2^{nd} intron, 3^{rd} exon, 3′ UTR, and any portion thereof, and the 5′ UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion thereof, of the endogenous Zm.SAMT gene; and
(b) obtaining at least one seed produced by said fertilizing of step (a).

61. The method of claim 60, wherein said method further comprises (c) growing said at least one seed obtained in step (b) to generate at least one progeny corn plant comprising said mutant allele.

62. The method of claim 61, wherein said at least one seed from step (b) is heterozygous for said mutant allele.

63. The method of claim 62, wherein said at least one seed from step (b) is homozygous for said mutant allele.

64. The method of any one of claims 60-63, wherein said female corn plant is homozygous for said mutant allele.

65. The method of any one of claims 60-63, wherein said female corn plant is heterozygous for said mutant allele.

66. The method of any one of claims 60-62, 64, or 65, wherein said male corn plant lacks said mutant allele.

67. The method of any one of claims 60-65, wherein said male corn plant is heterozygous for said mutant allele.

68. The method of any one of claims 60-65, wherein said male corn plant is homozygous for said mutant allele.

69. The method of any one of claims 61-68, wherein said at least one progeny corn plant has a shorter plant height and/or improved lodging resistance relative to a control plant lacking said mutant allele.

70. The method of any one of claims 61-68, wherein said at least one progeny corn plant has a shorter plant height and/or improved lodging resistance relative to said male corn plant.

71. The method of any one of claims 61-70, wherein said female corn plant is an inbred corn plant.

72. The method of any one of claims 61-70, wherein said female corn plant is a hybrid corn plant.

73. The method of any one of claims 61-70, wherein said male corn plant is an inbred corn plant.

74. The method of any one of claims 61-73, wherein said male corn plant is a hybrid corn plant.

75. The method of any one of claims 61-74, wherein said female corn plant is an elite corn plant line.

76. The method of any one of claims 61-75, wherein said male corn plant is an elite corn plant line.

77. The method of any one of claim 61-71, 73, 75, or 76, wherein said female corn plant is of a first inbred corn line or variety, and wherein said male corn plant is of a different, second inbred corn line or variety.

78. The method of any one of claims 61-77, wherein said female corn plant and said male corn plant are grown in a greenhouse or growth chamber.

79. The method of any one of claims 61-77, wherein said female corn plant and said male corn plant are grown outdoors.

80. The method of any one of claims 61-79, wherein said female corn plant has been detasseled.

81. The method of any one of claims 61-79, wherein said female corn plant is a cytoplasmically male sterile corn plant.

82. A modified corn plant part, corn cell, or corn tissue comprising a mutant allele of the endogenous GA20 oxidase_5 locus, wherein the mutant allele comprises a genome modification comprising a deletion of at least a portion of the transcription termination sequence of the endogenous Zm.SAMT gene, and wherein the mutant allele produces a RNA molecule comprising an antisense sequence complementary to all or part of the sense strand of the endogenous GA20 oxidase_5 gene.

83. A modified corn plant part, corn cell, or corn tissue comprising a mutant allele of the endogenous GA20 oxidase_5 locus, wherein the mutant allele comprises a genome modification comprising a deletion of at least a portion of the intergenic region between the endogenous GA20 oxidase_5 and Zm.SAMT genes, and wherein the mutant allele produces a RNA molecule comprising an antisense sequence complementary to all or part of the sense strand of the endogenous GA20 oxidase_5 gene.

84. A modified corn plant part, corn cell, or corn tissue comprising a mutant allele of the endogenous GA20 oxidase_5 locus, wherein the mutant allele comprises a genome modification comprising a deletion of at least a portion of one or more of the following: 5′UTR, 1^{st} exon, 1^{st} intron, 2^{nd} exon, 2^{nd} intron, 3^{rd} exon, 3′ UTR, and any portion thereof, and the 5′UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any portion thereof, of the endogenous Zm.SAMT gene.

85. A modified corn plant part, corn cell, or corn tissue comprising a mutant allele of the endogenous GA20 oxidase_5 locus, wherein the mutant allele comprises a genome modification which results in the transcription of an antisense strand of at least an exon, an intron, or an untranslated region (UTR) of the endogenous GA20 oxidase_5 gene, or any portion thereof.

86. A modified corn plant part, corn cell, or corn tissue comprising a mutant allele of the endogenous GA20 oxidase_5 locus, wherein the mutant allele comprises the Zm.SAMT gene promoter, or a functional part thereof, operably linked to at least one transcribable antisense sequence of at least an exon, intron or untranslated region (UTR) of the endogenous GA20 oxidase_5 gene, or any portion thereof.

87. A modified corn plant part, corn cell, or corn tissue comprising a mutant allele of the endogenous GA20 oxidase_5 locus, wherein the mutant allele comprises a sequence selected from the group consisting of SEQ ID NOs: 87-105.

88. A modified corn plant part, corn cell, or corn tissue comprising a mutant allele of the endogenous GA20 oxidase_5 locus, wherein the mutant allele comprises a first sequence and a second sequence; wherein the first sequence comprises one or more of the 5′UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene; wherein the first sequence and the second sequence are contiguous or separated only by an intervening sequence of fewer than 555 nucleotides.

89. A modified corn plant part, corn cell, or corn tissue comprising a mutant allele of the endogenous GA20 oxidase_5 locus, wherein the mutant allele comprises a genomic deletion relative to a wild type allele of the endogenous GA20 oxidase_5 locus, wherein the genomic deletion is flanked by a first sequence and a second sequence; wherein the first sequence comprises one or more of the 5′UTR, 1^{st} exon, 1^{st} intron, 2^{nd} exon, 2^{nd} intron, 3^{rd} exon, 3′ UTR, and any complementary sequence thereof, and any portion of the foregoing, of the endogenous Zm.GA20 oxidase_5 gene; and wherein the second sequence comprises one or more of the 5′UTR, 1^{st} exon, 1^{st} intron, 2^{nd} exon, 2^{nd} intron, 3^{rd} exon, 3^{rd} intron, 4^{th} exon, 4^{th} intron, 5^{th} exon, 5^{th} intron, 6^{th} exon, 6^{th} intron, 7^{th} exon, 7^{th} intron, 8^{th} exon, 3′ UTR, and any complementary sequence thereof, and any portion of the foregoing, of the endogenous Zm.SAMT gene.

90. A modified corn plant part, corn cell, or corn tissue comprising a mutant allele of the endogenous GA20 oxidase_5 locus, wherein the mutant allele comprises a genomic sequence comprising a first sequence and a second sequence; wherein the first sequence comprises at least 15 consecutive nucleotides of one or more of SEQ ID NOs: 11-18 and 59-66; wherein the second sequence comprises at least 15 consecutive nucleotides of one or more of SEQ ID NOs: 18-38 and 39-59; and wherein the genomic sequence is at least 50 consecutive nucleotides in length, and/or fewer than 9000 consecutive nucleotides in length.


SEQUENCE LISTING (105 sequences)

SEQ ID NO: 1 - DNA, 8800 bp, zea mays
taaatttgtgatccttgtgaagttgttatatcatgaattgtgaacttgttgcatttgtgatcttttgtcaactttgttgtattgtgaagtttgatatgtttaccgatcgtattttagatttcgatcgttaccggtgtattttccgcaccaaacttttgtttccgatgttttcgaaataccgatatcgtttccgtttctatagttacccttttcaattttatttccgattaaaaatatgaaaacggtaatggttttagtgtttatcgaccgttttcatctctaatcatccctgccggtgaagtttaatttttcccttggctaaagagatgcaagctgctgtaaaatacgttaaaacaggcaaggcagccccagcagccagcatcgcgtgcccgtctatgtacatcagtggatacgtagcatctctagtgagtaatataacgattgcatttggctggaggacgtatgttatataagtatgtcatttaccagttgcattagtatcttccctaactcctataataactctcttcgtggaatggacgtagacgtatgctatataagtattaaaaaatagttttttaagctggtgtcctcaattttgctattgttctcgtttttatctttagttgtgtcacaaatttaatccgtacaacaaatcaaaaataccatacccttcttatattaattttctaacataacatttgtttagatattttcagtcgtgaaaatacaattctaattctaacgtcgtagtatcaaatcaaaccatccagaatttgaccaagcttaattataaaaaatataaaatttatgatactgaatagatagcattagatttgttatataatatatttttataaaataccatttttatggtataaatattggtactcctttactttaaactatagatagttttgactaaggatgcaactagaattgcatcctcttttcactgcaccttcattagttttaatatttatttagatgggcccttgcaaactgtagatatcatctcttgcaacattctttctatagcaccacgaaaatgtattgcggctttgaaattataattgaattagttgtatcatttctttcaccgatgcgttaaattcaaaattaagtgttatatttcttcataatttgttaaatatatagaccctataatccaccattatttactataatagcatacattaacattggttttagcctacactacgacactcgaggcattgaattttcctctatcaaagaattatatgtgtagtagtattgttcttgacaaaaagggggattaaaattaaactaccaatattgatacttatcttatcacatccatgaatacaatcaacactcttacaaaagataagatacaagattaaaaagtaccatgataatacattaagattattagcaatgcattaaattaaataaatgtgcaagtgaatcatgattttagttttatctattttacttttaaaatatgatattctctgactacttctaagcataaatgtgattctaagtcatgaccgatcgtgcttattcagaaaaatgaaggagacacagatttctataaaaaaaggttgtcatgggactattgggtcaaccatcttattcatttgggaaaataagtttagaacacatcaacccattttagatgttgagtttggccctaatggtccattgaccttacttttgtgggttgacatagaccatctatcccaagttattgttgtgtcacattccctgatatcatgaatctatattttagctttccgttttcatatttttagtcgttacatattttttatccgcgtactagattaaaactctagttgttgcaatacattttgttcatttttttctatttcttctttactaacaacatattctagttcctagctacattcttaagtaccatagtgctataaacattttttatcctacattattccacttaagaaattgaattttctgcataaaaaaattatatgtccagtagtgttgtcttataaaagcataaagtgattaaaattaaaaccattattgatatcttatttttcaaaaaaaaatataagcttatagaaagtgaattaatttcatggtaaattaatatagtttaaattgaattattagtgttattactatgtttattatcaatgaaacatttttcatggttgatataacttagtgttacttattttagtattttttatataattctagttaacttttagtttttgatttaaaaaaacgagaattgtgtccttttgtggagtgagtataaagaaagtaatatctgttcatcataatttggttttttaaggtacgtgaaacttgctttatatttggactcaagctatgtctaaatacatagtaaaaaagcaatatttctagaaaagacaaaacatcttataatttagaatcaaggaaatatatagattttatgtgcagtgagaagccatttacaatggaacgttcaacgttgggccaatagatattttgcgatatgatgatgggcatatttttgcatggttgtccctccactagctatagtttgatgatacgatacgctgcacacaccattgggttgtaccatgttagtgtagcaacagtagaaacccaattgtggccgtgaaccatgataatactaggtagagtgctagctagaggtttcaggctattgatgcgtgaattaaactttctgttgtgttgcgaggaaacgagtattgtgaaatatttgaaacggttttttttgtgaaagatttgaaacggtatttttgttgtgaaataaagatcaaggctaaataaattcaaactaataaaacatattaattgacggcctgaagcccccgcccccatggccccatgccatagcatcaggtcccacatgacatgaggccgcgcctccctctatgttggctccctgccttcgccgttgtcgtcgctcccgaactccctctcctcccctgttacaaatacccccacccgcccggacagcttccctgcacactcgcagctcgcacatctcatggtgtcctaagaacggcaagagccagctctgcctagcagcagcgcacagccacatccatggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaacgagccggccgtctctttcgccggggcccgcgcggggttcgcccacgtggtgatcaggtggcagacatgtggcccacgggccccgcgccgccttccccatttttggacgaccctactgctactactactagtgtacatatgcaaaaaaatacatatatatataggtactttctctaatatttttatatataagcaaggcggcctggtgttcttttctttgttttgtcgacaactgtttgatcccatcctatggacgatggatagttcaatgtttgtacgtacgtaactactctctatagactagaatgggctcatgaaactggaccgatcgacacggacgtcacgtgcgtctggtaccggtagtgcaacgggtgccgaatgtttgctgggcccggacgagaatcgcttctcctcgtcctcggtcctcaccctgaacgaacgaataaggaaaatgctgcaccgaaagctccagacgtttccgaattccaaattccaaaaccccaaatcttcttgcttcacatcagtcttacccggttcatctgtgacaaaaaaaaaatagtgctagtttaggaactcaggtcgagattgaaggcaattgtggaggaatttaccctataatccttatgagaatttgagttcccaaactaactgagttggagcattcaacatttccctaaattttgtgcacatgtttctttgctatttatctttggacatgggacgatgggagacgcagatttaggggacccttcaattcagaacttcaggtgcacaaaccgaggttggcttgcctgcattcttgtttcggacatgcccaactaggccactactcactaccttcatctgagataccaattgctgacctaaatgacaagtatacacttacatttcagtgatagctgcaacaaaaaaaaaaatcttaccgcattttatctctgcattctgcatgccgcatcctgaacattacgtatctttcccggtgctctgttgcgttctcacgcagttgatggcatgcagtcttgcgccaccgaatccagtgtactggtcgtggtgacttgtcgcacagacagcagcccggcagcaccaagcgtgtcactgtaaactgttgggcgttaaacaacaacttgcacaacagctcaaatatggcatatgctatccgacaaactgaacaaggtgcccaattgatctgaatgtacctgtgatttccagcactatcgtacagcaacgttgtcaaaacaagtggggtggggttggggacagattttttcgataaagaagcttttataaaaaataacaatgatacaaatcctgggttatagatgtacagaaagcacgaagcacgaaagtccagtccaaagcacgtttttttgcctggtactagcccgatccggccggcacgaataagcgggtcgagctcggacgggaagctaagcacgacggactagcccgacacgacccgtttacctctaatcccgttaaacccgcttttttgcactaaaccgtgcttaccgaaccgtttagcccgttttttggcctgatttttcgtgcttaacgggccaggctcggacaaggaaacaagcccgcgggcttagacgatccggcctggttttttaaccgtgcctagcgggtcgagcccaaaataggtcgggcttcactgggcccgagccgggcgacccgtttggccatctctaacctgagtacaacttccgacttctgcaaaatacatacagccaaataaaagaaaagtaaaaagatctaaaaaatctcagctagaaacaatcaatccgaagactagatcgcctcccataagtctggaaaaaagagaccatggccacttgcaccaagatgtgtgcattattctacggttacaccaaagatttttgttctttaagggctagtttgggaaccataattttcaaagggatttctattttcctaaggaaaattagttcatttttccataagaaattagaaatccattggaaaattgtggttctcaaactagccctaagcgtcgaaaagaaccatatgcatatctagagacaaaattcctctaatttctattcaggcttcagcacatatacttcacgtgcttgcgtcaagttccttgggccgccacatggacttatggacttctcgacgcagcgaaagccgtcgttgcccttggtgtagctaggtcatccgcacctcccactggccagtggccactgcagggacttggccatgggcttgtttggttcagcttttttctgaccagcttttctgaaaatctggctgtgtgaagaatctggctgtgagagaatctgagtatcattacgattacgtgtggatgaagataaagttgttcatagggctcaggatctagaaagtgacggattcctactattacaatgactcaaccgattatgtgtttatgttgattttggatgatttttgccccaacaaattttatagaagctggctgaaaagctgagcgtttggcagtccacaacagtttttggtggccagaagctgccagaagccgatacaaacagggtccatgctttccatttcgtttaccgtgtacgcggtgtccctcacaatcaatcagtttaccttgtggctccaacacacatcaacctcggcacaacaacactgtgaatcattttcggcggtccatattattttcgacggttcatccctggccgccgaaaattgtctgttattttcggcggcttgacctagccgtcgaaaataggctgctattttcggcggccaaatccgagccgccgaaaataaggcttttaaaaaccgtcggctccttcttcttctctgttctttctctcctctcccaaagcccgccgccgctcacccgccgctcgctcgccgggctgcccgccactccgccgtcgtcgtcgagccaccgcatcgagaggtaaattttttttgcgtgttttattccttattttcggcggttgatatttaggcgccgccaaaattagtgtatatttgtaattgtgtttgtttaattactatttgtaattagtattattgtttaattcgatttcattaatgtattagtggtatatgtgatttagggattaggggcatattgtatttaggcattaatttcatattaatatgtggtattattatattattggttttaatagtacattatattggtacgtagaatagttgcattattagtgtttgtggtacttagtttgacttgat

SEQ ID NO: 2 - DNA, 1522 bp, Zea mays
gcacactcgcagctcgcacatctcatggtgtcctaagaacggcaagagccagctctgcctagcagcagcgcacagccacatccatggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaacgagccggccgtctctttcgccggggcccgcgcggggttcgcccacgtggtgatcaggtggcagacatgtggcccacgggccccgcgccgccttccccatttttggacgaccctactgctactactactagtgtacatatgcaaaaaaatacatatatatataggtactttctctaatatttttatatataagcaaggcggcctggtgttcttttctttgttttgtcgacaactgtttgatcccatcctatggacgatggatagttcaatgtttgtac

SEQ ID NO: 3 - DNA, 1161 bp, Zea mays
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 4 - protein, 386 aa, Zea mays
MDASPTPPLPLRAPTPSIDLPAGKDRADAAANKAAAVFDLRREPKIPEPFLWPHEEARPTSAAELEVPVVDVGVLRNGDGAGLRRAAAQVAAACATHGFFQVCGHGVDAALGRAALDGASDFFRLPLAEKQRARRVPGTVSGYTSAHADRFASKLPWKETLSFGFHDGAAAPVVVDYFTGTLGQDFEPVGRVYQRYCEEMKELSLTIMELLELSLGVERGYYREFFEDSRSIMRCNYYPPCPVPERTLGTGPHCDPTALTILLQDDVGGLEVLVDGEWRPVRPVPGAMVINIGDTFMALSNGRYKSCLHRAVVNRRQERQSLAFFLCPREDRVVRPPASAAPRQYPDFTWADLMRFTQRHYRADTRTLDAFTRWLSHGPAAAAPCT

SEQ ID NO: 5 - DNA, 8859 bp, Zea mays
cctattttgtgtctaatactcttcttatattaattgtttggtcaaactttagataaatttgactaatgatgcaattaaaactgcatcacctttactaaggtactgctttatatgtttcgacaaaattttcaattattctctatgtgttttaatctttgcgctacacctccattgatttaaatactcatttattttaaaccataacttaaattatatcggatctttgcatcctttctatggcaccatacatgaatcgatattttggctgcaaatttttaatcatgttagttttagcattttttcatatccatgtgttaagtttgaatcatgtgttgtttttatataatttattgaaaatatagatcctaaacttcactaatacttacaacaatagcatcatcatgtgttttaatccacgccacaacactcaaggcattgaattttcttctaccaaagagttgtatgtgtgtattgttctttaaaaaatagagtgattataattaaactaccagtattcatatgtaaaatgtatagacatctaaaataaaatttgcaaaaaacattgttgcagactttcaatataattaagaatgggttttagggtcatgatatatggtttgttaaagaaacttgtttttttttgcaattgataaactataaaatacattttcactattgtgtgcatatgtacttggtatacatagtggcatatatcatttttgtttactttgaggtttgaattatctatgttaaaattggataacatagatacattggtgtgcgtcctttggcccatttacttgactgaggagcaatactataaagtaaaacatatttggatattttatcttaaactcctagcataatattgatttaattatgaacaaatatatgtttaggtgatagtttcatgggtggtaaactatataagaaggcttaccatgatctttgcaaactctaggctatgaaagagttccatgatttgtcttagaagcatagacaaaacagtgataatgatctaaatcacacttatggcactgatgaccatatatgcaaagctaaatgcatgttaagttgtattatatcatatgtttacaatgactatcgcatataacgaggaatacattgtctatatagatagctattactgtagtagtgccaaatgttggacaacatgaatcataatcttcaaacctagagaaattgtagtcagtcgtacacatatcgtctagtaagttgtctatactttttatttattgtatcaaattttattgttatcttgcttgcttgtttgtttgtaccatagacacaatatggtcaaaaagtggtcaatcgattcgaagaagattgcaattgacgagtgctaacagttgatccttttgttgtgcacgctagcggagtagcatgaaaagagtaaaatatgaaattagcgttctaaactgtttgtgctataggtacttcgtatttaatggagtgactaactataggaaggtgagagctcagaagtcagcaccctcacacagagttctagagttagtggtcatcgaaccacgacaaactacatgatgagcagaagaggcaacatcaagactatgatcaatagtttcgggtcaatgaatgacatcgtgatgagtatttatctaactatatagaacaacaacacatgatgttttaagtaagttcaactgatcttctattgctatctttaagtatttaacgtagcgaataatgttttatctatttcattcataaataatgttgtgacaaaaggggataaccatcacttttaccatgttctagataccacaaccatctccaccatcataatgggttcttcattggtgcttggacctcaaataatcatatctatagccaacttagctcaattctaataaaattaggcaacttggcttcattgtagcaaaaatagccaacttagctcaattttatctaaacttagctaatctagcacaacttagatcaatattaggaaaaactaatcaatctaatctagctcaactatagcgaaagatagatattgtagcataacttagtagatctatctcaaattttagcaaaaactaatcaatttagataaactctataaaattttaatcattatgacttatttccaactaattgtaacttgcatgatttttatgttccttctttataattagcaacacctaaagacacgaatgatgaggggtctaacgcattcattaaccagttgttaaataatactctaggtagatgataagaactctaattattctatgaatctaagctaaaagatgtttaatatttaagtattggtgtttattatgttatttagaacgattcatgttacttaaagatttgttatgatttttaaatatgattatgataatttatgtggtgtggattaacttgtgaacatatgtgatgtagatgaatatgtatgttgtggatggaaccatatgaatatatatacacactcatatactattcgttggtgtaggtaaagcttcatccatcggtaattactaaatggtcttcagtcattaccactaggtgaagcttcacacgaccgataattattgaagaacgctcattaatttccggtaatggcttattggccttcactagtcggtgaaaattagctatttttataccaataaaaattagctaatatatgtaaaccaggtctaatttttatgggcctcttaccgaccaaaattgattagattattgttacaatagttttagtcaaaagctagctatgctataaaaattttgaattaaagtgagtttcgtaataaaaattgcatacttttaaaataaaataattaaaaaacagtttttagaaatacaatcaaacaccttatgctataaaaaaattgtaatgtacctacaaatatataatactttactttaaaataggcctgtgccttctcggctctatatgggctgcctccaacgaagcgccatggccatgggctccactgtgtcgggtcccacatgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaactgatttctgtgaaatttctttgttttaacaagaaaacagaaaaattacatgaggaaaacgctccatttatttcaacaagaaaaaaatacatgaaacagaaggagaaaaaacgtgttcgttctatcattttcacacgagaaaaaaaaacatagaaaacagaaaaactccccgcgttcagatgagctcaagaaaatggaacgacacggacgtcacccgcgtcttgtagcagtgggcgcacgggtgccgaatgtttgctgggcccccaagagaatcgcttctcctcacgctgaatgaatgaatcaacgagggaaacgctgcaccctgagttccagacgtttccgaattccaaacgtttttgtggcgtgcgtccatggggcgcccccaaacttcggacgtttccggcgctccaacaaatcttctcgcttcacacgtcaccgtcgtcccggattcatttgcctcgtcgctccaccattcgctgctctcctctccacgtactcttaccctgacctttgggaaagaactgaacattcgagatgcacaacagttcaaatataacatatgcagcacaagatcgttcgactgctatccgacaagccaacaacgtgcccagtagaactgaatgtacctgtgatttccagcactaacttacagcaacgttgtgaaaaaacaaaaacgaaaacaaacggcagaaaaaacagatgtattgttctacagttacaccaaatattttctggtcctttcagcaccaacaagagccatacgcatatctagaagacaaaattcctctaatttcacccctacgtggtagcagttcctcctcaacacagttcacgtgctagcgtcgagttctttgggccgccacatcgacttctcgacgcagagcaggccctcgctgcccttggtgtaggtcatccgcacctcccactgcacggacttggccatgctctccagctcatttatcgtgtccgcggtgtccctcacgatcagcttgccctgtggcctcagtacacggtcgacctcggcgaaaactgcagccagtttgcatctgtaaacaggcaacacagatttttagtatctaaaacactgcaggcaaacgccacaggttttagtcgcaagaagcaataaaagcatgcaaacaatgctacgtgtacgtatcaaaggaacatgtcaaaactcgttgcatgaacgatcattgatgtttccttgctgaactagtcacatcagtctgcttcaacttctgggtttcactagtagatataccagaagggtagaataatgtgaagagcaagaaatacagacctctttctgagctttgagaacagatggtccgcgtgcagaaggtcatacgttcttgggtaagtgctgaaagactcgcaccagtcatggtacatgccaaacaaaccgcgctcgtagatgatgggcagcgtgtctggtgaatcgatcggcacgatattcatgacccagaccttttggtccctcagagctgcagcaaaactgccatgcaacaatgtaaagcattagtcaagaagaaggtgtacagtgcatttctccttgtcaacagtcttcagtaacaaaaaaaaagtgttatgcttgactgaatctttcaaagaaatatgcttgatgacttatggtggacaagttgcctgttatagtgttatgttttaattaactatgtgccagcttgggtaactagtagttatgtagtgtgatctgaattaccaaaatataaataaataaataaacatgcccaagaaactacgaaaaccatttacttaccctccatagacagctctcatgtccatgacatttctcactttggaccagtcaattcccatgccattcacatacgatttacttacaacccgtttccagtgggcattatctgcctcaaaatcttcatttgcaggctttccatagacaccaaccttggaaccatcaatccagaaaggggtcttctcaagcctttgcggccataactctggccattttgatcctcggacttttgagccaccaggcagtttgtgcatgcatgcttccaacggtacattcctgcaaatcaaaaggctgtgtaagcaaagcagagaagcacttttctccattgaaaatatactcttctcaaagaaccgaaaccataccaagcagcatctgcatcatcagattccttgcacaatggcgggctgttttcagatcttttctcatagcaaatattgtccattggtttctgatatatgaccataccaacttggtttaacttatccttagtcttgttgaccatcttccagcacatggactttgtcaaagtagacatggctgaaaagggtatgtggccacatgttatgttagaaataaaattcaattttgaacagttggtccatagcatgtattttgaacaaatgcaatccttctccatccatgaaagaagttgacccttcatacttaggattattcagtactttcactcatgtctgctgaatttgttctcttggtagttgctatacaagaaagggggaagtacagagtagctaaacttatacaagctatagtctgatatttgtatgaaacataaattttggtatggatgtcttattaaaatgggaggttgtataatatttttctagcctacctcaacttgcttgagactaaaaggctttgttgttgttgttgaggctgtatggtgctttgactttacaaatcaagttatcagctaccctacttatggatatacacctctcataaaatgatggtaagaagtttcgatatgtcacattaacataagaacttcattcagttagggtacaacgaagttaagtagttacggaaataccattccaaatctcaacatcctctgggagcttttggtaaacaggagtggcagaccagacaaagtaaccaccagggcgtaacaagcggttcaattccagcaaaagcatgccacctaaaagtagcgagccagcaataagattcagttctatagcaaatcaataaatgaaaggaggacatgtcaatatgtaaccagcaggacaaaccttcgatgtgccaaggga

SEQ ID NO: 6 - DNA, 1733 bp, Zea mays
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaa

SEQ ID NO: 7 - DNA, 1392 bp, Zea mays
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 8 - protein, 463 aa, Zea mays
MRPRLPPNVPSLPSSLSLLANSLSSPVTNTPTRPDSFPAYLQLAHLMVSQERQEPAVPSSSSSSAKRAATSMDASPAPPLLLRAPTPSPSIDLPAGKDKADAAASKAGAAVFDLRREPKIPAPFLWPQEEARPSSAAELEVPMVDVGVLRNGDRAGLRRAAAQVAAACATHGFFQVCGHGVDAALGRAALDGASDFFRLPLAEKQRARRVPGTVSGYTSAHADRFAAKLPWKETLSFGYHDGAASPVVVDYFVGTLGQDFEPMGWVYQRYCEEMKELSLTIMELLELSLGVELRGYYREFFEDSRSIMRCNYYPPCPEPERTLGTGPHCDPTALTILLQDDVGGLEVLVDGEWRPVRPVPGAMVINIGDTFMALSNGRYKSCLHRAVVNQRRARRSLAFFLCPREDRVVRPPASAAPRRYPDFTWADLMRFTQRHYRADTRTLDAFTRWLSHGPAQAAAPPCT

SEQ ID NO: 9 - DNA, 9067 bp, Zea mays
ggtaatggcttattggccttcactagtcggtgaaaattagctatttttataccaataaaaattagctaatatatgtaaaccaggtctaatttttatgggcctcttaccgaccaaaattgattagattattgttacaatagttttagtcaaaagctagctatgctataaaaattttgaattaaagtgagtttcgtaataaaaattgcatacttttaaaataaaataattaaaaaacagtttttagaaatacaatcaaacaccttatgctataaaaaaattgtaatgtacctacaaatatataatactttactttaaaataggcctgtgccttctcggctctatatgggctgcctccaacgaagcgccatggccatgggctccactgtgtcgggtcccacatgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaactgatttctgtgaaatttctttgttttaacaagaaaacagaaaaattacatgaggaaaacgctccatttatttcaacaagaaaaaaatacatgaaacagaaggagaaaaaacgtgttcgttctatcattttcacacgagaaaaaaaaacatagaaaacagaaaaactccccgcgttcagatgagctcaagaaaatggaacgacacggacgtcacccgcgtcttgtagcagtgggcgcacgggtgccgaatgtttgctgggcccccaagagaatcgcttctcctcacgctgaatgaatgaatcaacgagggaaacgctgcaccctgagttccagacgtttccgaattccaaacgtttttgtggcgtgcgtccatggggcgcccccaaacttcggacgtttccggcgctccaacaaatcttctcgcttcacacgtcaccgtcgtcccggattcatttgcctcgtcgctccaccattcgctgctctcctctccacgtactcttaccctgacctttgggaaagaactgaacattcgagatgcacaacagttcaaatataacatatgcagcacaagatcgttcgactgctatccgacaagccaacaacgtgcccagtagaactgaatgtacctgtgatttccagcactaacttacagcaacgttgtgaaaaaacaaaaacgaaaacaaacggcagaaaaaacagatgtattgttctacagttacaccaaatattttctggtcctttcagcaccaacaagagccatacgcatatctagaagacaaaattcctctaatttcacccctacgtggtagcagttcctcctcaacacagttcacgtgctagcgtcgagttctttgggccgccacatcgacttctcgacgcagagcaggccctcgctgcccttggtgtaggtcatccgcacctcccactgcacggacttggccatgctctccagctcatttatcgtgtccgcggtgtccctcacgatcagcttgccctgtggcctcagtacacggtcgacctcggcgaaaactgcagccagtttgcatctgtaaacaggcaacacagatttttagtatctaaaacactgcaggcaaacgccacaggttttagtcgcaagaagcaataaaagcatgcaaacaatgctacgtgtacgtatcaaaggaacatgtcaaaactcgttgcatgaacgatcattgatgtttccttgctgaactagtcacatcagtctgcttcaacttctgggtttcactagtagatataccagaagggtagaataatgtgaagagcaagaaatacagacctctttctgagctttgagaacagatggtccgcgtgcagaaggtcatacgttcttgggtaagtgctgaaagactcgcaccagtcatggtacatgccaaacaaaccgcgctcgtagatgatgggcagcgtgtctggtgaatcgatcggcacgatattcatgacccagaccttttggtccctcagagctgcagcaaaactgccatgcaacaatgtaaagcattagtcaagaagaaggtgtacagtgcatttctccttgtcaacagtcttcagtaacaaaaaaaaagtgttatgcttgactgaatctttcaaagaaatatgcttgatgacttatggtggacaagttgcctgttatagtgttatgttttaattaactatgtgccagcttgggtaactagtagttatgtagtgtgatctgaattaccaaaatataaataaataaataaacatgcccaagaaactacgaaaaccatttacttaccctccatagacagctctcatgtccatgacatttctcactttggaccagtcaattcccatgccattcacatacgatttacttacaacccgtttccagtgggcattatctgcctcaaaatcttcatttgcaggctttccatagacaccaaccttggaaccatcaatccagaaaggggtcttctcaagcctttgcggccataactctggccattttgatcctcggacttttgagccaccaggcagtttgtgcatgcatgcttccaacggtacattcctgcaaatcaaaaggctgtgtaagcaaagcagagaagcacttttctccattgaaaatatactcttctcaaagaaccgaaaccataccaagcagcatctgcatcatcagattccttgcacaatggcgggctgttttcagatcttttctcatagcaaatattgtccattggtttctgatatatgaccataccaacttggtttaacttatccttagtcttgttgaccatcttccagcacatggactttgtcaaagtagacatggctgaaaagggtatgtggccacatgttatgttagaaataaaattcaattttgaacagttggtccatagcatgtattttgaacaaatgcaatccttctccatccatgaaagaagttgacccttcatacttaggattattcagtactttcactcatgtctgctgaatttgttctcttggtagttgctatacaagaaagggggaagtacagagtagctaaacttatacaagctatagtctgatatttgtatgaaacataaattttggtatggatgtcttattaaaatgggaggttgtataatatttttctagcctacctcaacttgcttgagactaaaaggctttgttgttgttgttgaggctgtatggtgctttgactttacaaatcaagttatcagctaccctacttatggatatacacctctcataaaatgatggtaagaagtttcgatatgtcacattaacataagaacttcattcagttagggtacaacgaagttaagtagttacggaaataccattccaaatctcaacatcctctgggagcttttggtaaacaggagtggcagaccagacaaagtaaccaccagggcgtaacaagcggttcaattccagcaaaagcatgccacctaaaagtagcgagccagcaataagattcagttctatagcaaatcaataaatgaaaggaggacatgtcaatatgtaaccagcaggacaaaccttcgatgtgccaagggaccctgcagcgagcgcaatgaatgacatcaaagactctgctggggtacggaagtctcttggtgcccatcacagctgatattgctggaattcccctttctaatgcaaattgtacttgagcttcatgctcatcttttggagcaaaagacatggtaagcgcatctctatcaaacatgtagcctccaaagctggcaactccacaaccaacatccaatatgacacggcttcgtttgccccatgcaatatcaggtagtgcctgtgaatggcagtttaatcagcatagggtgaaagcaagtgtgataacattaagttcaaagacgcaacatgaaacctcaatatcatggtacagtactcagcttatttgctatattaatgtagggatgagactaaaaaaaaggaaagttttatccgccagaatgagaggctgaaaatacagggatgactaatgttgcttagtctagcacatacaaagttacaaactgtcctcaacacctgcagatttctatatggtgctatttgacaaattatgtttgtggaatagtataacaataaatcaatcactgatgctcaaaagtgtgaggcagacaagtacaagtctaaggagtgactaatatgagatgctgggatgattataatatacctgctgaatagtatcaatatagtggagggcaccattcttgaactgagtcccacccccagggaacaagagatagtcacctgagaccttaacccaattttgatgccccttgtactctgcaagcctagtgtgaggaacattgctgtaccatacctgcaaaaaacagcacagcgtggtaataagcaaacacgcatcttggtcagctaaagatgattcggtgttgtacaattcagaataaacagaatcaccttgtccctgctctttggccactcaattggccgtttatatccttctgggagtggaacaaggcaggtaggaggctcctcagggcaatgcctctcacgatgttcataatgtttggtagttcgaagcttcttgatagccttctcgttgtcaaggcaaggtatgtaatctgttgaggcactactattacataatttccaggtatagctggttgcatcgcctgaagacttcggtaacgcttggacttccttttcattctttgactctgcagcctgtgtggggaatgaaccgttctgggtatttgactccttcagaagctctgattgggccccatcaggaaatacctcgttggagtttgagctctgatccttctctccattttcttccacgttctcttctattttaggttgctcctcctgagcggcatcgccatcaggcttctcttcttgaccgtccttactctcaccatcagttttttcatcaccactctcatttgtgatttcattgtctttcttctccacactcttctccccatcatcattcttcgtttcatctgacagcccttctgattttccatttgcgtcatcaaacatatccttggtctccgctttctctaccggcacttccagctctttctcttcaggcttctcattgaaattctcttgctcagaagcatcctgtttattcggttcctctgggactgtggcatcattgttgtcggtgtcctcaaatttctcggacccttcaccagcaatgccaggtgatgcctcctgcgacgccccgaaattgacaggcgcaggctgttgcttcaccaccggcttcttattggacgaaatctccagcgggaagacagtggacgaggtcatcatccacgcgccgactaggcagagcgccacaaagagcacgaccgtggtggttgtgcagaacgacgacgacgacgacgatgaggacggccggcggccgtccatctttccacctcggccaaatgccatcagtgcctggcgaacatgtaccagagcaccagccttcacgtggtttatctccaccaacaaccacggctggaccaacagcccccccaaaatcgcagctttgtctgccctgtgtatgctgttacgacttacgaccgcgcggcaccgaagcaaaccacaaaaaagaaactaaatcgctgcgggtttaaatcaagctgctggatctagagaaggaaacggagatctactcaagcgacaccgaaaggacggtcccggattggtgctattagcatcttgtttcctactacagcgtctctttgaagaaaagaacgcggagaaatcaccccgtaaggccaagcatggaaagaaattcagtaaagcgcgggcattaaaacccccccgtcctgctccttccgcggagagctacggcaccttccaattgagctactagctctcagctgggcgcagaacccgcactaataaatggcggattccatccagaaaaaagaagaagaagaaacagctaaataatccagcacctcgctcgcctcctcgttcgctagctcatcggcggggaaggacgggaccagctccgctggatccacgccagcaagcgggtgcaaggagagagggaacggagcagcaatgcggaggcggtaggctggtacctcgccggaaccgaccggagcggtcgcggtggccctccgagtggatctcgaggcgaggcgcgtccttgggttctctgcctccccgcactgggctcgccgcgttataaaggcaggcgggcagcgcagtggaggtgggagagtggagtgcaacctgtttgtgttagtgtgcccagagcggaagcggaggagatgggtccgcgttataaagggcctgtttggttcagcttttttctgaccagcttttttaagaatctggctgtgaggagaatctggctgtggggagaaactgagtattat

SEQ ID NO: 10 - DNA, 9067 bp, Zea mays
ataatactcagtttctccccacagccagattctcctcacagccagattcttaaaaaagctggtcagaaaaaagctgaaccaaacaggccctttataacgcggacccatctcctccgcttccgctctgggcacactaacacaaacaggttgcactccactctcccacctccactgcgctgcccgcctgcctttataacgcggcgagcccagtgcggggaggcagagaacccaaggacgcgcctcgcctcgagatccactcggagggccaccgcgaccgctccggtcggttccggcgaggtaccagcctaccgcctccgcattgctgctccgttccctctctccttgcacccgcttgctggcgtggatccagcggagctggtcccgtccttccccgccgatgagctagcgaacgaggaggcgagcgaggtgctggattatttagctgtttcttcttcttcttttttctggatggaatccgccatttattagtgcgggttctgcgcccagctgagagctagtagctcaattggaaggtgccgtagctctccgcggaaggagcaggacgggggggttttaatgcccgcgctttactgaatttctttccatgcttggccttacggggtgatttctccgcgttcttttcttcaaagagacgctgtagtaggaaacaagatgctaatagcaccaatccgggaccgtcctttcggtgtcgcttgagtagatctccgtttccttctctagatccagcagcttgatttaaacccgcagcgatttagtttcttttttgtggtttgcttcggtgccgcgcggtcgtaagtcgtaacagcatacacagggcagacaaagctgcgattttgggggggctgttggtccagccgtggttgttggtggagataaaccacgtgaaggctggtgctctggtacatgttcgccaggcactgatggcatttggccgaggtggaaagatggacggccgccggccgtcctcatcgtcgtcgtcgtcgtcgttctgcacaaccaccacggtcgtgctctttgtggcgctctgcctagtcggcgcgtggatgatgacctcgtccactgtcttcccgctggagatttcgtccaataagaagccggtggtgaagcaacagcctgcgcctgtcaatttcggggcgtcgcaggaggcatcacctggcattgctggtgaagggtccgagaaatttgaggacaccgacaacaatgatgccacagtcccagaggaaccgaataaacaggatgcttctgagcaagagaatttcaatgagaagcctgaagagaaagagctggaagtgccggtagagaaagcggagaccaaggatatgtttgatgacgcaaatggaaaatcagaagggctgtcagatgaaacgaagaatgatgatggggagaagagtgtggagaagaaagacaatgaaatcacaaatgagagtggtgatgaaaaaactgatggtgagagtaaggacggtcaagaagagaagcctgatggcgatgccgctcaggaggagcaacctaaaatagaagagaacgtggaagaaaatggagagaaggatcagagctcaaactccaacgaggtatttcctgatggggcccaatcagagcttctgaaggagtcaaatacccagaacggttcattccccacacaggctgcagagtcaaagaatgaaaaggaagtccaagcgttaccgaagtcttcaggcgatgcaaccagctatacctggaaattatgtaatagtagtgcctcaacagattacataccttgccttgacaacgagaaggctatcaagaagcttcgaactaccaaacattatgaacatcgtgagaggcattgccctgaggagcctcctacctgccttgttccactcccagaaggatataaacggccaattgagtggccaaagagcagggacaaggtgattctgtttattctgaattgtacaacaccgaatcatctttagctgaccaagatgcgtgtttgcttattaccacgctgtgctgttttttgcaggtatggtacagcaatgttcctcacactaggcttgcagagtacaaggggcatcaaaattgggttaaggtctcaggtgactatctcttgttccctgggggtgggactcagttcaagaatggtgccctccactatattgatactattcagcaggtatattataatcatcccagcatctcatattagtcactccttagacttgtacttgtctgcctcacacttttgagcatcagtgattgatttattgttatactattccacaaacataatttgtcaaatagcaccatatagaaatctgcaggtgttgaggacagtttgtaactttgtatgtgctagactaagcaacattagtcatccctgtattttcagcctctcattctggcggataaaactttccttttttttagtctcatccctacattaatatagcaaataagctgagtactgtaccatgatattgaggtttcatgttgcgtctttgaacttaatgttatcacacttgctttcaccctatgctgattaaactgccattcacaggcactacctgatattgcatggggcaaacgaagccgtgtcatattggatgttggttgtggagttgccagctttggaggctacatgtttgatagagatgcgcttaccatgtcttttgctccaaaagatgagcatgaagctcaagtacaatttgcattagaaaggggaattccagcaatatcagctgtgatgggcaccaagagacttccgtaccccagcagagtctttgatgtcattcattgcgctcgctgcagggtcccttggcacatcgaaggtttgtcctgctggttacatattgacatgtcctcctttcatttattgatttgctatagaactgaatcttattgctggctcgctacttttaggtggcatgcttttgctggaattgaaccgcttgttacgccctggtggttactttgtctggtctgccactcctgtttaccaaaagctcccagaggatgttgagatttggaatggtatttccgtaactacttaacttcgttgtaccctaactgaatgaagttcttatgttaatgtgacatatcgaaacttcttaccatcattttatgagaggtgtatatccataagtagggtagctgataacttgatttgtaaagtcaaagcaccatacagcctcaacaacaacaacaaagccttttagtctcaagcaagttgaggtaggctagaaaaatattatacaacctcccattttaataagacatccataccaaaatttatgtttcatacaaatatcagactatagcttgtataagtttagctactctgtacttccccctttcttgtatagcaactaccaagagaacaaattcagcagacatgagtgaaagtactgaataatcctaagtatgaagggtcaacttctttcatggatggagaaggattgcatttgttcaaaatacatgctatggaccaactgttcaaaattgaattttatttctaacataacatgtggccacatacccttttcagccatgtctactttgacaaagtccatgtgctggaagatggtcaacaagactaaggataagttaaaccaagttggtatggtcatatatcagaaaccaatggacaatatttgctatgagaaaagatctgaaaacagcccgccattgtgcaaggaatctgatgatgcagatgctgcttggtatggtttcggttctttgagaagagtatattttcaatggagaaaagtgcttctctgctttgcttacacagccttttgatttgcaggaatgtaccgttggaagcatgcatgcacaaactgcctggtggctcaaaagtccgaggatcaaaatggccagagttatggccgcaaaggcttgagaagacccctttctggattgatggttccaaggttggtgtctatggaaagcctgcaaatgaagattttgaggcagataatgcccactggaaacgggttgtaagtaaatcgtatgtgaatggcatgggaattgactggtccaaagtgagaaatgtcatggacatgagagctgtctatggagggtaagtaaatggttttcgtagtttcttgggcatgtttatttatttatttatattttggtaattcagatcacactacataactactagttacccaagctggcacatagttaattaaaacataacactataacaggcaacttgtccaccataagtcatcaagcatatttctttgaaagattcagtcaagcataacactttttttttgttactgaagactgttgacaaggagaaatgcactgtacaccttcttcttgactaatgctttacattgttgcatggcagttttgctgcagctctgagggaccaaaaggtctgggtcatgaatatcgtgccgatcgattcaccagacacgctgcccatcatctacgagcgcggtttgtttggcatgtaccatgactggtgcgagtctttcagcacttacccaagaacgtatgaccttctgcacgcggaccatctgttctcaaagctcagaaagaggtctgtatttcttgctcttcacattattctacccttctggtatatctactagtgaaacccagaagttgaagcagactgatgtgactagttcagcaaggaaacatcaatgatcgttcatgcaacgagttttgacatgttcctttgatacgtacacgtagcattgtttgcatgcttttattgcttcttgcgactaaaacctgtggcgtttgcctgcagtgttttagatactaaaaatctgtgttgcctgtttacagatgcaaactggctgcagttttcgccgaggtcgaccgtgtactgaggccacagggcaagctgatcgtgagggacaccgcggacacgataaatgagctggagagcatggccaagtccgtgcagtgggaggtgcggatgacctacaccaagggcagcgagggcctgctctgcgtcgagaagtcgatgtggcggcccaaagaactcgacgctagcacgtgaactgtgttgaggaggaactgctaccacgtaggggtgaaattagaggaattttgtcttctagatatgcgtatggctcttgttggtgctgaaaggaccagaaaatatttggtgtaactgtagaacaatacatctgttttttctgccgtttgttttcgtttttgttttttcacaacgttgctgtaagttagtgctggaaatcacaggtacattcagttctactgggcacgttgttggcttgtcggatagcagtcgaacgatcttgtgctgcatatgttatatttgaactgttgtgcatctcgaatgttcagttctttcccaaaggtcagggtaagagtacgtggagaggagagcagcgaatggtggagcgacgaggcaaatgaatccgggacgacggtgacgtgtgaagcgagaagatttgttggagcgccggaaacgtccgaagtttgggggcgccccatggacgcacgccacaaaaacgtttggaattcggaaacgtctggaactcagggtgcagcgtttccctcgttgattcattcattcagcgtgaggagaagcgattctcttgggggcccagcaaacattcggcacccgtgcgcccactgctacaagacgcgggtgacgtccgtgtcgttccattttcttgagctcatctgaacgcggggagtttttctgttttctatgtttttttttctcgtgtgaaaatgatagaacgaacacgttttttctccttctgtttcatgtatttttttcttgttgaaataaatggagcgttttcctcatgtaatttttctgttttcttgttaaaacaaagaaatttcacagaaatcagtttaatttttctcagaaaaaacacagaggaaggaaaagaatcccacgttcacggtcttagtacaaacattgaagtatccatggtccatagaataatgggatcaaacagttgtggacaaaacaagagaagggcaccgggccgccttgcttttatatataatatatagaaaatacgtatatatatatatatatatgtactcttgcatatgtacactagtagcacagcagtagggccagcgtcaaaaaatggcaaggcggcgcggggcccgcgggccgcgtgtctggccacctgcccagctgggcgcgcacgtggggcgaaagagacggccttggcccggctcgctaggtgcagggaggcgccgccgcctgggccgggccgtgggagagccagcgggtgaaggcgtccagcgtgcgggtgtcggcgcggtagtggcgctgcgtgaagcgcatgaggtcggcccaggtgaagtccgggtagcgccgcggcgcagcactggccggcgggcgcaccacccggtcctcgcgcgggcacaggaagaaggccagcgaccgccgcgcccgccgctggttcaccaccgcgcggtgcaggcagctcttgtacctcccgttcgacagcgcctgcatggcatgcgggttagcacagtgcacaagaatcatcgtgtcgggctgtctcattgttgcggtgcgcccggcggcgggccaggccgcgacaaccggacgcgtgcaagtgcctgccacccacacggccaagggcccgagctgtttggagccccggcgtggtgtgggcgccgcgtgccggatcgccagcgaaaccgcgacaaaagtgcaagcggaaacggcgggggcaacagcagccaccacgtaccggtgacccgatccaacccaacccgactccaaccaaccttgtactatatactatacattatactccgcgcgacggccctccgcgcccgtgcggcatcgcatagggccgctcgcgtggcgcggcggatcagacgggatgggcgccgcaccgagcgtcggtctcggtcgtcgacccatttctcggccatggacaccgagagggggaaaccgacaaggccgcgcgcacctggacacggccacccgccgaaagcgttgccctgcccggcacccatcgtatcgtctccccgccacgtccccgtcgacgtccccgccgccgcgcaagcctctccgcaacgctccaacgccacaaaccaacagggaacttgctaccgctagccgaccccctcggtccgttctcttgtcgcaactctcccccggaagcctcgatcggcctgggcgcggccgcggatagactagagagagcgcgccgcggccgcgtgtacctgccgtagcccgccgagataggaaccaactcgaacgcatgtctctattgcatttgcatcggcatcgctatgtatgtactagctacatgacatgcctacgtgtgcgtgctgtaataacctgcccgtttaggtccactaggattcgggaaataacagcacagagagagctcaactgaatatacacacaggacaagagcggccaaggaaaacagaggagcgagcgctttcgtttcgttaccatgaaggtgtcgccgatgttgatgaccatggcgcccgggacgggccggacggggcgccactcaccgtccaccagcacctccagcccgcccacgtcgtcctgcaggaggatggtgagcgccgtggggtcgcagtgcgggcccgtgcccagcgtgcgctccggctccgggcacggcgggtagtagttgcaccgcatgatggaccggctgtcctcgaagaactcccggtagtagccgcgcagctccacgcccaggctcagctccagcagctccatgatcgtcagcgacagctccttcatctcctcgcagtacctctggtacacccacctgccacacatgcatgggacgggacgggggttcatacgtctcccgagtcccgactcgaccgcacgccgacgtgcggtaaatgtaaaccggcgctccttcttactaccttacttaccccattggctcgaaatcctggccgagggtgccgacgaagtagtccacgacgacaggcgacgcggcgccgtcgtggtagccgaacgacagggtctccttccaggggagcttggccgcgaaccggtcggcgtgcgcgctcgtgtacccggacacggtgccggggacgcgccgggcgcgctgcttctcggcgagcggcagccggaagaagtcgctggcgccgtccagcgcggcgcgccccagcgccgcgtccacgccgtgcccgcacacctggaagaacccgtgcgtcgcgcacgccgcggccacctgcgccgcggcgcgccgcagccccgcgcggtcgccattgcgcagcacgcccacgtccaccatcggcacctccagctccgcggccgaggacggccgcgcctcttcctgcggccacaggaatggcgcggggatcttgggctcccggcgcaggtcgaacacggccgcgccggccttgctggccgccgcgtcggccttgtccttgccagcggggaggtcaatgctggggctgggagtgggggcgcggaggaggagcggcggggccgggctggcgtccatggacgtggctgcgcgcttggcgctgctgctgctgctgctaggcacagctggctcttgtcgttcctgcgacaccatgagatgtgcgagctgcaagtatgcagggaagctgtccgggcgggtgggggtatttgtaacaggggaggacagggagtttgcgagcaacgacaaagacgaaggcagggagggaacatttggagggaggcgcggcctcatgtgggacccgacacagtggagcccatggccatggcgcttcgttggaggcagcccatatagagccgagaaggcacaggcctattttaaagtaaagtattatatatttgtaggtacattacaatttttttatagcataaggtgtttgattgtatttctaaaaactgttttttaattattttattttaaaagtatgcaatttttattacgaaactcactttaattcaaaatttttatagcatagctagcttttgactaaaactattgtaacaataatctaatcaattttggtcggtaagaggcccataaaaattagacctggtttacatatattagctaatttttattggtataaaaatagctaattttcaccgactagtgaaggccaataagccattacc

SEQ ID NO: 11 - DNA, 398 bp, Zea mays
ggtaatggcttattggccttcactagtcggtgaaaattagctatttttataccaataaaaattagctaatatatgtaaaccaggtctaatttttatgggcctcttaccgaccaaaattgattagattattgttacaatagttttagtcaaaagctagctatgctataaaaattttgaattaaagtgagtttcgtaataaaaattgcatacttttaaaataaaataattaaaaaacagtttttagaaatacaatcaaacaccttatgctataaaaaaattgtaatgtacctacaaatatataatactttactttaaaataggcctgtgccttctcggctctatatgggctgcctccaacgaagcgccatggccatgggctccactgtgtcgggtcccac

SEQ ID NO: 12 - DNA, 791 bp, Zea mays
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggg

SEQ ID NO: 13 - DNA, 115 bp, Zea mays
gtaagtaaggtagtaagaaggagcgccggtttacatttaccgcacgtcggcgtgcggtcgagtcgggactcgggagacgtatgaacccccgtcccgtcccatgcatgtgtggcag

SEQ ID NO: 14 - DNA, 325 bp, Zea mays
gtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatg

SEQ ID NO: 15 - DNA, 966 bp, Zea mays
gtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcag

SEQ ID NO: 16 - DNA, 276 bp, Zea mays
gcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 17 - DNA, 309 bp, Zea mays
cgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttc

SEQ ID NO: 18 - DNA, 556 bp, Zea mays
cttcctctgtgttttttctgagaaaaattaaactgatttctgtgaaatttctttgttttaacaagaaaacagaaaaattacatgaggaaaacgctccatttatttcaacaagaaaaaaatacatgaaacagaaggagaaaaaacgtgttcgttctatcattttcacacgagaaaaaaaaacatagaaaacagaaaaactccccgcgttcagatgagctcaagaaaatggaacgacacggacgtcacccgcgtcttgtagcagtgggcgcacgggtgccgaatgtttgctgggcccccaagagaatcgcttctcctcacgctgaatgaatgaatcaacgagggaaacgctgcaccctgagttccagacgtttccgaattccaaacgtttttgtggcgtgcgtccatggggcgcccccaaacttcggacgtttccggcgctccaacaaatcttctcgcttcacacgtcaccgtcgtcccggattcatttgcctcgtcgctccaccattcgctgctctcctctccacgtactcttaccctgacctttgggaaagaactg

SEQ ID NO: 19 - DNA, 405 bp, Zea mays
aacattcgagatgcacaacagttcaaatataacatatgcagcacaagatcgttcgactgctatccgacaagccaacaacgtgcccagtagaactgaatgtacctgtgatttccagcactaacttacagcaacgttgtgaaaaaacaaaaacgaaaacaaacggcagaaaaaacagatgtattgttctacagttacaccaaatattttctggtcctttcagcaccaacaagagccatacgcatatctagaagacaaaattcctctaatttcacccctacgtggtagcagttcctcctcaacacagttcacgtgctagcgtcgagttctttgggccgccacatcgacttctcgacgcagagcaggccctcgctgcccttggtgtaggtcatccgcacctcccactgc

SEQ ID NO: 20 - DNA, 217 bp, Zea mays
tcacgtgctagcgtcgagttctttgggccgccacatcgacttctcgacgcagagcaggccctcgctgcccttggtgtaggtcatccgcacctcccactgcacggacttggccatgctctccagctcatttatcgtgtccgcggtgtccctcacgatcagcttgccctgtggcctcagtacacggtcgacctcggcgaaaactgcagccagtttgcat

SEQ ID NO: 21 - DNA, 254 bp, Zea mays
ctgtaaacaggcaacacagatttttagtatctaaaacactgcaggcaaacgccacaggttttagtcgcaagaagcaataaaagcatgcaaacaatgctacgtgtacgtatcaaaggaacatgtcaaaactcgttgcatgaacgatcattgatgtttccttgctgaactagtcacatcagtctgcttcaacttctgggtttcactagtagatataccagaagggtagaataatgtgaagagcaagaaatacagac

SEQ ID NO: 22 - DNA, 195 bp, Zea mays
ctctttctgagctttgagaacagatggtccgcgtgcagaaggtcatacgttcttgggtaagtgctgaaagactcgcaccagtcatggtacatgccaaacaaaccgcgctcgtagatgatgggcagcgtgtctggtgaatcgatcggcacgatattcatgacccagaccttttggtccctcagagctgcagcaaaa

SEQ ID NO: 23 - DNA, 282 bp, Zea mays
ctgccatgcaacaatgtaaagcattagtcaagaagaaggtgtacagtgcatttctccttgtcaacagtcttcagtaacaaaaaaaaagtgttatgcttgactgaatctttcaaagaaatatgcttgatgacttatggtggacaagttgcctgttatagtgttatgttttaattaactatgtgccagcttgggtaactagtagttatgtagtgtgatctgaattaccaaaatataaataaataaataaacatgcccaagaaactacgaaaaccatttacttac

SEQ ID NO: 24 - DNA, 273 bp, Zea mays
cctccatagacagctctcatgtccatgacatttctcactttggaccagtcaattcccatgccattcacatacgatttacttacaacccgtttccagtgggcattatctgcctcaaaatcttcatttgcaggctttccatagacaccaaccttggaaccatcaatccagaaaggggtcttctcaagcctttgcggccataactctggccattttgatcctcggacttttgagccaccaggcagtttgtgcatgcatgcttccaacggtacattc

SEQ ID NO: 25 - DNA, 86 bp, Zea mays
ctgcaaatcaaaaggctgtgtaagcaaagcagagaagcacttttctccattgaaaatatactcttctcaaagaaccgaaaccatac

SEQ ID NO: 26 - DNA, 175 bp, Zea mays
caagcagcatctgcatcatcagattccttgcacaatggcgggctgttttcagatcttttctcatagcaaatattgtccattggtttctgatatatgaccataccaacttggtttaacttatccttagtcttgttgaccatcttccagcacatggactttgtcaaagtagacatgg

SEQ ID NO: 27 - DNA, 514 bp, Zea mays
ctgaaaagggtatgtggccacatgttatgttagaaataaaattcaattttgaacagttggtccatagcatgtattttgaacaaatgcaatccttctccatccatgaaagaagttgacccttcatacttaggattattcagtactttcactcatgtctgctgaatttgttctcttggtagttgctatacaagaaagggggaagtacagagtagctaaacttatacaagctatagtctgatatttgtatgaaacataaattttggtatggatgtcttattaaaatgggaggttgtataatatttttctagcctacctcaacttgcttgagactaaaaggctttgttgttgttgttgaggctgtatggtgctttgactttacaaatcaagttatcagctaccctacttatggatatacacctctcataaaatgatggtaagaagtttcgatatgtcacattaacataagaacttcattcagttagggtacaacgaagttaagtagttacggaaatac

SEQ ID NO: 28 - DNA, 111 bp, Zea mays
cattccaaatctcaacatcctctgggagcttttggtaaacaggagtggcagaccagacaaagtaaccaccagggcgtaacaagcggttcaattccagcaaaagcatgccac

SEQ ID NO: 29 - DNA, 111 bp, Zea mays
ttccagcaaaagcatgccacctaaaagtagcgagccagcaataagattcagttctatagcaaatcaataaatgaaaggaggacatgtcaatatgtaaccagcaggacaaac

SEQ ID NO: 30 - DNA, 271 bp, Zea mays
cttcgatgtgccaagggaccctgcagcgagcgcaatgaatgacatcaaagactctgctggggtacggaagtctcttggtgcccatcacagctgatattgctggaattcccctttctaatgcaaattgtacttgagcttcatgctcatcttttggagcaaaagacatggtaagcgcatctctatcaaacatgtagcctccaaagctggcaactccacaaccaacatccaatatgacacggcttcgtttgccccatgcaatatcaggtagtgc

SEQ ID NO: 31 - DNA, 384 bp, Zea mays
ctgtgaatggcagtttaatcagcatagggtgaaagcaagtgtgataacattaagttcaaagacgcaacatgaaacctcaatatcatggtacagtactcagcttatttgctatattaatgtagggatgagactaaaaaaaaggaaagttttatccgccagaatgagaggctgaaaatacagggatgactaatgttgcttagtctagcacatacaaagttacaaactgtcctcaacacctgcagatttctatatggtgctatttgacaaattatgtttgtggaatagtataacaataaatcaatcactgatgctcaaaagtgtgaggcagacaagtacaagtctaaggagtgactaatatgagatgctgggatgattataatatac

SEQ ID NO: 32 - DNA, 150 bp, Zea mays
ctgctgaatagtatcaatatagtggagggcaccattcttgaactgagtcccacccccagggaacaagagatagtcacctgagaccttaacccaattttgatgccccttgtactctgcaagcctagtgtgaggaacattgctgtaccatac

SEQ ID NO: 33 - DNA, 95 bp, Zea mays
ctgcaaaaaacagcacagcgtggtaataagcaaacacgcatcttggtcagctaaagatgattcggtgttgtacaattcagaataaacagaatcac

SEQ ID NO: 34 - DNA, 987 bp, Zea mays
cttgtccctgctctttggccactcaattggccgtttatatccttctgggagtggaacaaggcaggtaggaggctcctcagggcaatgcctctcacgatgttcataatgtttggtagttcgaagcttcttgatagccttctcgttgtcaaggcaaggtatgtaatctgttgaggcactactattacataatttccaggtatagctggttgcatcgcctgaagacttcggtaacgcttggacttccttttcattctttgactctgcagcctgtgtggggaatgaaccgttctgggtatttgactccttcagaagctctgattgggccccatcaggaaatacctcgttggagtttgagctctgatccttctctccattttcttccacgttctcttctattttaggttgctcctcctgagcggcatcgccatcaggcttctcttcttgaccgtccttactctcaccatcagttttttcatcaccactctcatttgtgatttcattgtctttcttctccacactcttctccccatcatcattcttcgtttcatctgacagcccttctgattttccatttgcgtcatcaaacatatccttggtctccgctttctctaccggcacttccagctctttctcttcaggcttctcattgaaattctcttgctcagaagcatcctgtttattcggttcctctgggactgtggcatcattgttgtcggtgtcctcaaatttctcggacccttcaccagcaatgccaggtgatgcctcctgcgacgccccgaaattgacaggcgcaggctgttgcttcaccaccggcttcttattggacgaaatctccagcgggaagacagtggacgaggtcatcatccacgcgccgactaggcagagcgccacaaagagcacgaccgtggtggttgtgcagaacgacgacgacgacgacgatgaggacggccggcggccgtccatctttccacctcggccaaatgccat

SEQ ID NO: 35 - DNA, 142 bp, Zea mays
cagtgcctggcgaacatgtaccagagcaccagccttcacgtggtttatctccaccaacaaccacggctggaccaacagcccccccaaaatcgcagctttgtctgccctgtgtatgctgttacgacttacgaccgcgcggcac

SEQ ID NO: 36 - DNA, 503 bp, Zea mays
cgaagcaaaccacaaaaaagaaactaaatcgctgcgggtttaaatcaagctgctggatctagagaaggaaacggagatctactcaagcgacaccgaaaggacggtcccggattggtgctattagcatcttgtttcctactacagcgtctctttgaagaaaagaacgcggagaaatcaccccgtaaggccaagcatggaaagaaattcagtaaagcgcgggcattaaaacccccccgtcctgctccttccgcggagagctacggcaccttccaattgagctactagctctcagctgggcgcagaacccgcactaataaatggcggattccatccagaaaaaagaagaagaagaaacagctaaataatccagcacctcgctcgcctcctcgttcgctagctcatcggcggggaaggacgggaccagctccgctggatccacgccagcaagcgggtgcaaggagagagggaacggagcagcaatgcggaggcggtaggctggtacc

SEQ ID NO: 37 - DNA, 171 bp, Zea mays
tcgccggaaccgaccggagcggtcgcggtggccctccgagtggatctcgaggcgaggcgcgtccttgggttctctgcctccccgcactgggctcgccgcgttataaaggcaggcgggcagcgcagtggaggtgggagagtggagtgcaacctgtttgtgttagtgtgccca

SEQ ID NO: 38 - DNA, 125 bp, Zea mays
gagcggaagcggaggagatgggtccgcgttataaagggcctgtttggttcagcttttttctgaccagcttttttaagaatctggctgtgaggagaatctggctgtggggagaaactgagtattat

SEQ ID NO: 39 - DNA, 125 bp, Zea mays
ataatactcagtttctccccacagccagattctcctcacagccagattcttaaaaaagctggtcagaaaaaagctgaaccaaacaggccctttataacgcggacccatctcctccgcttccgctc

SEQ ID NO: 40 - DNA, 171 bp, Zea mays
tgggcacactaacacaaacaggttgcactccactctcccacctccactgcgctgcccgcctgcctttataacgcggcgagcccagtgcggggaggcagagaacccaaggacgcgcctcgcctcgagatccactcggagggccaccgcgaccgctccggtcggttccggcga

SEQ ID NO: 41 - DNA, 503 bp, Zea mays
ggtaccagcctaccgcctccgcattgctgctccgttccctctctccttgcacccgcttgctggcgtggatccagcggagctggtcccgtccttccccgccgatgagctagcgaacgaggaggcgagcgaggtgctggattatttagctgtttcttcttcttcttttttctggatggaatccgccatttattagtgcgggttctgcgcccagctgagagctagtagctcaattggaaggtgccgtagctctccgcggaaggagcaggacgggggggttttaatgcccgcgctttactgaatttctttccatgcttggccttacggggtgatttctccgcgttcttttcttcaaagagacgctgtagtaggaaacaagatgctaatagcaccaatccgggaccgtcctttcggtgtcgcttgagtagatctccgtttccttctctagatccagcagcttgatttaaacccgcagcgatttagtttcttttttgtggtttgcttcg

SEQ ID NO: 42 - DNA, 142 bp, Zea mays
gtgccgcgcggtcgtaagtcgtaacagcatacacagggcagacaaagctgcgattttgggggggctgttggtccagccgtggttgttggtggagataaaccacgtgaaggctggtgctctggtacatgttcgccaggcactg

SEQ ID NO: 43 - DNA, 987 bp, Zea mays
atggcatttggccgaggtggaaagatggacggccgccggccgtcctcatcgtcgtcgtcgtcgtcgttctgcacaaccaccacggtcgtgctctttgtggcgctctgcctagtcggcgcgtggatgatgacctcgtccactgtcttcccgctggagatttcgtccaataagaagccggtggtgaagcaacagcctgcgcctgtcaatttcggggcgtcgcaggaggcatcacctggcattgctggtgaagggtccgagaaatttgaggacaccgacaacaatgatgccacagtcccagaggaaccgaataaacaggatgcttctgagcaagagaatttcaatgagaagcctgaagagaaagagctggaagtgccggtagagaaagcggagaccaaggatatgtttgatgacgcaaatggaaaatcagaagggctgtcagatgaaacgaagaatgatgatggggagaagagtgtggagaagaaagacaatgaaatcacaaatgagagtggtgatgaaaaaactgatggtgagagtaaggacggtcaagaagagaagcctgatggcgatgccgctcaggaggagcaacctaaaatagaagagaacgtggaagaaaatggagagaaggatcagagctcaaactccaacgaggtatttcctgatggggcccaatcagagcttctgaaggagtcaaatacccagaacggttcattccccacacaggctgcagagtcaaagaatgaaaaggaagtccaagcgttaccgaagtcttcaggcgatgcaaccagctatacctggaaattatgtaatagtagtgcctcaacagattacataccttgccttgacaacgagaaggctatcaagaagcttcgaactaccaaacattatgaacatcgtgagaggcattgccctgaggagcctcctacctgccttgttccactcccagaaggatataaacggccaattgagtggccaaagagcagggacaag

SEQ ID NO: 44 - DNA, 95 bp, Zea mays
gtgattctgtttattctgaattgtacaacaccgaatcatctttagctgaccaagatgcgtgtttgcttattaccacgctgtgctgttttttgcag

SEQ ID NO: 45 - DNA, 150 bp, Zea mays
gtatggtacagcaatgttcctcacactaggcttgcagagtacaaggggcatcaaaattgggttaaggtctcaggtgactatctcttgttccctgggggtgggactcagttcaagaatggtgccctccactatattgatactattcagcag

SEQ ID NO: 46 - DNA, 384 bp, Zea mays
gtatattataatcatcccagcatctcatattagtcactccttagacttgtacttgtctgcctcacacttttgagcatcagtgattgatttattgttatactattccacaaacataatttgtcaaatagcaccatatagaaatctgcaggtgttgaggacagtttgtaactttgtatgtgctagactaagcaacattagtcatccctgtattttcagcctctcattctggcggataaaactttccttttttttagtctcatccctacattaatatagcaaataagctgagtactgtaccatgatattgaggtttcatgttgcgtctttgaacttaatgttatcacacttgctttcaccctatgctgattaaactgccattcacag

SEQ ID NO: 47 - DNA, 271 bp, Zea mays
gcactacctgatattgcatggggcaaacgaagccgtgtcatattggatgttggttgtggagttgccagctttggaggctacatgtttgatagagatgcgcttaccatgtcttttgctccaaaagatgagcatgaagctcaagtacaatttgcattagaaaggggaattccagcaatatcagctgtgatgggcaccaagagacttccgtaccccagcagagtctttgatgtcattcattgcgctcgctgcagggtcccttggcacatcgaag

SEQ ID NO: 48 - DNA, 111 bp, Zea mays
gtttgtcctgctggttacatattgacatgtcctcctttcatttattgatttgctatagaactgaatcttattgctggctcgctacttttaggtggcatgcttttgctggaa

SEQ ID NO: 49 - DNA, 111 bp, Zea mays
gtggcatgcttttgctggaattgaaccgcttgttacgccctggtggttactttgtctggtctgccactcctgtttaccaaaagctcccagaggatgttgagatttggaatg

SEQ ID NO: 50 - DNA, 514 bp, Zea mays
gtatttccgtaactacttaacttcgttgtaccctaactgaatgaagttcttatgttaatgtgacatatcgaaacttcttaccatcattttatgagaggtgtatatccataagtagggtagctgataacttgatttgtaaagtcaaagcaccatacagcctcaacaacaacaacaaagccttttagtctcaagcaagttgaggtaggctagaaaaatattatacaacctcccattttaataagacatccataccaaaatttatgtttcatacaaatatcagactatagcttgtataagtttagctactctgtacttccccctttcttgtatagcaactaccaagagaacaaattcagcagacatgagtgaaagtactgaataatcctaagtatgaagggtcaacttctttcatggatggagaaggattgcatttgttcaaaatacatgctatggaccaactgttcaaaattgaattttatttctaacataacatgtggccacatacccttttcag

SEQ ID NO: 51 - DNA, 175 bp, Zea mays
ccatgtctactttgacaaagtccatgtgctggaagatggtcaacaagactaaggataagttaaaccaagttggtatggtcatatatcagaaaccaatggacaatatttgctatgagaaaagatctgaaaacagcccgccattgtgcaaggaatctgatgatgcagatgctgcttg

SEQ ID NO: 52 - DNA, 86 bp, Zea mays
gtatggtttcggttctttgagaagagtatattttcaatggagaaaagtgcttctctgctttgcttacacagccttttgatttgcag

SEQ ID NO: 53 - DNA, 273 bp, Zea mays
gaatgtaccgttggaagcatgcatgcacaaactgcctggtggctcaaaagtccgaggatcaaaatggccagagttatggccgcaaaggcttgagaagacccctttctggattgatggttccaaggttggtgtctatggaaagcctgcaaatgaagattttgaggcagataatgcccactggaaacgggttgtaagtaaatcgtatgtgaatggcatgggaattgactggtccaaagtgagaaatgtcatggacatgagagctgtctatggagg

SEQ ID NO: 54 - DNA, 282 bp, Zea mays
gtaagtaaatggttttcgtagtttcttgggcatgtttatttatttatttatattttggtaattcagatcacactacataactactagttacccaagctggcacatagttaattaaaacataacactataacaggcaacttgtccaccataagtcatcaagcatatttctttgaaagattcagtcaagcataacactttttttttgttactgaagactgttgacaaggagaaatgcactgtacaccttcttcttgactaatgctttacattgttgcatggcag

SEQ ID NO: 55 - DNA, 195 bp, Zea mays
ttttgctgcagctctgagggaccaaaaggtctgggtcatgaatatcgtgccgatcgattcaccagacacgctgcccatcatctacgagcgcggtttgtttggcatgtaccatgactggtgcgagtctttcagcacttacccaagaacgtatgaccttctgcacgcggaccatctgttctcaaagctcagaaagag

SEQ ID NO: 56 - DNA, 254 bp, Zea mays
gtctgtatttcttgctcttcacattattctacccttctggtatatctactagtgaaacccagaagttgaagcagactgatgtgactagttcagcaaggaaacatcaatgatcgttcatgcaacgagttttgacatgttcctttgatacgtacacgtagcattgtttgcatgcttttattgcttcttgcgactaaaacctgtggcgtttgcctgcagtgttttagatactaaaaatctgtgttgcctgtttacag

SEQ ID NO: 57 - DNA, 217 bp, Zea mays
atgcaaactggctgcagttttcgccgaggtcgaccgtgtactgaggccacagggcaagctgatcgtgagggacaccgcggacacgataaatgagctggagagcatggccaagtccgtgcagtgggaggtgcggatgacctacaccaagggcagcgagggcctgctctgcgtcgagaagtcgatgtggcggcccaaagaactcgacgctagcacgtga

SEQ ID NO: 58 - DNA, 405 bp, Zea mays
gcagtgggaggtgcggatgacctacaccaagggcagcgagggcctgctctgcgtcgagaagtcgatgtggcggcccaaagaactcgacgctagcacgtgaactgtgttgaggaggaactgctaccacgtaggggtgaaattagaggaattttgtcttctagatatgcgtatggctcttgttggtgctgaaaggaccagaaaatatttggtgtaactgtagaacaatacatctgttttttctgccgtttgttttcgtttttgttttttcacaacgttgctgtaagttagtgctggaaatcacaggtacattcagttctactgggcacgttgttggcttgtcggatagcagtcgaacgatcttgtgctgcatatgttatatttgaactgttgtgcatctcgaatgtt

SEQ ID NO: 59 - DNA, 556 bp, Zea mays
cagttctttcccaaaggtcagggtaagagtacgtggagaggagagcagcgaatggtggagcgacgaggcaaatgaatccgggacgacggtgacgtgtgaagcgagaagatttgttggagcgccggaaacgtccgaagtttgggggcgccccatggacgcacgccacaaaaacgtttggaattcggaaacgtctggaactcagggtgcagcgtttccctcgttgattcattcattcagcgtgaggagaagcgattctcttgggggcccagcaaacattcggcacccgtgcgcccactgctacaagacgcgggtgacgtccgtgtcgttccattttcttgagctcatctgaacgcggggagtttttctgttttctatgtttttttttctcgtgtgaaaatgatagaacgaacacgttttttctccttctgtttcatgtatttttttcttgttgaaataaatggagcgttttcctcatgtaatttttctgttttcttgttaaaacaaagaaatttcacagaaatcagtttaatttttctcagaaaaaacacagaggaag

SEQ ID NO: 60 - DNA, 309 bp, Zea mays
gaaaagaatcccacgttcacggtcttagtacaaacattgaagtatccatggtccatagaataatgggatcaaacagttgtggacaaaacaagagaagggcaccgggccgccttgcttttatatataatatatagaaaatacgtatatatatatatatatatgtactcttgcatatgtacactagtagcacagcagtagggccagcgtcaaaaaatggcaaggcggcgcggggcccgcgggccgcgtgtctggccacctgcccagctgggcgcgcacgtggggcgaaagagacggccttggcccggctcg

SEQ ID NO: 61 - DNA, 276 bp, Zea mays
ctaggtgcagggaggcgccgccgcctgggccgggccgtgggagagccagcgggtgaaggcgtccagcgtgcgggtgtcggcgcggtagtggcgctgcgtgaagcgcatgaggtcggcccaggtgaagtccgggtagcgccgcggcgcagcactggccggcgggcgcaccacccggtcctcgcgcgggcacaggaagaaggccagcgaccgccgcgcccgccgctggttcaccaccgcgcggtgcaggcagctcttgtacctcccgttcgacagcgc

SEQ ID NO: 62 - DNA, 966 bp, Zea mays
ctgcatggcatgcgggttagcacagtgcacaagaatcatcgtgtcgggctgtctcattgttgcggtgcgcccggcggcgggccaggccgcgacaaccggacgcgtgcaagtgcctgccacccacacggccaagggcccgagctgtttggagccccggcgtggtgtgggcgccgcgtgccggatcgccagcgaaaccgcgacaaaagtgcaagcggaaacggcgggggcaacagcagccaccacgtaccggtgacccgatccaacccaacccgactccaaccaaccttgtactatatactatacattatactccgcgcgacggccctccgcgcccgtgcggcatcgcatagggccgctcgcgtggcgcggcggatcagacgggatgggcgccgcaccgagcgtcggtctcggtcgtcgacccatttctcggccatggacaccgagagggggaaaccgacaaggccgcgcgcacctggacacggccacccgccgaaagcgttgccctgcccggcacccatcgtatcgtctccccgccacgtccccgtcgacgtccccgccgccgcgcaagcctctccgcaacgctccaacgccacaaaccaacagggaacttgctaccgctagccgaccccctcggtccgttctcttgtcgcaactctcccccggaagcctcgatcggcctgggcgcggccgcggatagactagagagagcgcgccgcggccgcgtgtacctgccgtagcccgccgagataggaaccaactcgaacgcatgtctctattgcatttgcatcggcatcgctatgtatgtactagctacatgacatgcctacgtgtgcgtgctgtaataacctgcccgtttaggtccactaggattcgggaaataacagcacagagagagctcaactgaatatacacacaggacaagagcggccaaggaaaacagaggagcgagcgctttcgtttcgttac

SEQ ID NO: 63 - DNA, 325 bp, Zea mays
catgaaggtgtcgccgatgttgatgaccatggcgcccgggacgggccggacggggcgccactcaccgtccaccagcacctccagcccgcccacgtcgtcctgcaggaggatggtgagcgccgtggggtcgcagtgcgggcccgtgcccagcgtgcgctccggctccgggcacggcgggtagtagttgcaccgcatgatggaccggctgtcctcgaagaactcccggtagtagccgcgcagctccacgcccaggctcagctccagcagctccatgatcgtcagcgacagctccttcatctcctcgcagtacctctggtacacccac

SEQ ID NO: 64 - DNA, 115 bp, Zea mays
ctgccacacatgcatgggacgggacgggggttcatacgtctcccgagtcccgactcgaccgcacgccgacgtgcggtaaatgtaaaccggcgctccttcttactaccttacttac

SEQ ID NO: 65 - DNA, 791 bp, Zea mays
cccattggctcgaaatcctggccgagggtgccgacgaagtagtccacgacgacaggcgacgcggcgccgtcgtggtagccgaacgacagggtctccttccaggggagcttggccgcgaaccggtcggcgtgcgcgctcgtgtacccggacacggtgccggggacgcgccgggcgcgctgcttctcggcgagcggcagccggaagaagtcgctggcgccgtccagcgcggcgcgccccagcgccgcgtccacgccgtgcccgcacacctggaagaacccgtgcgtcgcgcacgccgcggccacctgcgccgcggcgcgccgcagccccgcgcggtcgccattgcgcagcacgcccacgtccaccatcggcacctccagctccgcggccgaggacggccgcgcctcttcctgcggccacaggaatggcgcggggatcttgggctcccggcgcaggtcgaacacggccgcgccggccttgctggccgccgcgtcggccttgtccttgccagcggggaggtcaatgctggggctgggagtgggggcgcggaggaggagcggcggggccgggctggcgtccatggacgtggctgcgcgcttggcgctgctgctgctgctgctaggcacagctggctcttgtcgttcctgcgacaccatgagatgtgcgagctgcaagtatgcagggaagctgtccgggcgggtgggggtatttgtaacaggggaggacagggagtttgcgagcaacgacaaagacgaaggcagggagggaacatttggagggaggcgcggcctcat

SEQ ID NO: 66 - DNA, 398 bp, Zea mays
gtgggacccgacacagtggagcccatggccatggcgcttcgttggaggcagcccatatagagccgagaaggcacaggcctattttaaagtaaagtattatatatttgtaggtacattacaatttttttatagcataaggtgtttgattgtatttctaaaaactgttttttaattattttattttaaaagtatgcaatttttattacgaaactcactttaattcaaaatttttatagcatagctagcttttgactaaaactattgtaacaataatctaatcaattttggtcggtaagaggcccataaaaattagacctggtttacatatattagctaatttttattggtataaaaatagctaattttcaccgactagtgaaggccaataagccattacc

SEQ ID NO: 67 - DNA, 877 bp, Zea mays
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttcccc

SEQ ID NO: 68 - protein, 1227 aa, Lachnospiraceae bacterium ND2006
SKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 69 - protein, 1229 aa, Lachnospiraceae bacterium ND2006
ASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 70 - DNA, 30 bp, Solanum lycopersicum
ggatctaagaagagaagaattaaacaagat

SEQ ID NO: 71 - DNA, 30 bp, Solanum lycopersicum; identical to SEQ ID NO: 70

SEQ ID NO: 72 - DNA, 33 bp, Solanum tuberosum
atgggtagcaaaaagaggcgtatcaagcaggac

SEQ ID NO: 73 - DNA, 30 bp, Solanum tuberosum
ggatctaagaagcgtaggatcaagcaagat

SEQ ID NO: 74 - DNA, 200 bp, Zea mays
cggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 75 - DNA, 23 bp, Zea mays
aggacaccgacaacaatgatgcc

SEQ ID NO: 76 - DNA, 23 bp, Zea mays
ggtccactaggattcgggaaata

SEQ ID NO: 77 - DNA, 23 bp, Zea mays
gagccaatggggtaagtaaggta

SEQ ID NO: 78 - DNA, 23 bp, Zea mays
gttaccatgaaggtgtcgccgat

SEQ ID NO: 79 - DNA, 23 bp, Zea mays
gtccaataagaagccggtggtga

SEQ ID NO: 80 - DNA, 23 bp, Zea mays
cacctcggccaaatgccatcagt

SEQ ID NO: 81 - DNA, 23 bp, Zea mays
gttgagctctctctgtgctgtta

SEQ ID NO: 82 - DNA, 23 bp, Zea mays
ctaggattcgggaaataacagca

SEQ ID NO: 83 - DNA, 23 bp, Zea mays
cctcggccaaatgccatcagtgc

SEQ ID NO: 84 - DNA, 23 bp, Zea mays
cgtggtttatctccaccaacaac

SEQ ID NO: 85 - DNA, 19 bp, Artificial Sequence
  PCR primer1
aggtgccgatggtggacgt

SEQ ID NO: 86 - DNA, 19 bp, Artificial Sequence
  PCR primer2
agcctaccgcctccgcatt

SEQ ID NO: 87 - DNA, 60 bp, Zea mays
gcggccgtccatctttccacctcggccaaagtgcctggcgaacatgtaccagagcaccag

SEQ ID NO: 88 - DNA, 60 bp, Zea mays
ggccgtccatctttccacctcggccaaatgtcagtgcctggcgaacatgtaccagagcac

SEQ ID NO: 89 - DNA, 60 bp, Zea mays
ggccgtccatctttccacctcggccaaatggtgcctggcgaacatgtaccagagcaccag

SEQ ID NO: 90 - DNA, 60 bp, Zea mays
gagtggcgccccgtccggcccgtcccgggcttcttattggacgaaatctccagcgggaag

SEQ ID NO: 91 - DNA, 60 bp, Zea mays
ccggcccgtcccgggcgccatggtcatcaagtgcctggcgaacatgtaccagagcaccag

SEQ ID NO: 92 - DNA, 60 bp, Zea mays
gtccggcccgtcccgggcgccatggtcatcggcttcttattggacgaaatctccagcggg

SEQ ID NO: 93 - DNA, 60 bp, Zea mays
gtccggcccgtcccgggcgccatggtcatcttattggacgaaatctccagcgggaagaca

SEQ ID NO: 94 - DNA, 60 bp, Zea mays
cgtccggcccgtcccgggcgccatggtcatgcttcttattggacgaaatctccagcggga

SEQ ID NO: 95 - DNA, 60 bp, Zea mays
ctgtgtgtatattcagttgagctctctctgcacggctggaccaacagcccccccaaaatc

SEQ ID NO: 96 - DNA, 60 bp, Zea mays
cttggccgctcttgtcctgtgtgtatattcggtgtcctcaaatttctcggacccttcacc

SEQ ID NO: 97 - DNA, 60 bp, Zea mays
tgtatattcagttgagctctctctgtgctggttgtcggtgtcctcaaatttctcggaccc

SEQ ID NO: 98 - DNA, 60 bp, Zea mays
tatattcagttgagctctctctgtgctgtttgttgtcggtgtcctcaaatttctcggacc

SEQ ID NO: 99 - DNA, 60 bp, Zea mays
atattcagttgagctctctctgtgctgttagttgtcggtgtcctcaaatttctcggaccc

SEQ ID NO: 100 - DNA, 60 bp, Zea mays
attcagttgagctctctctgtgctgttattgtcggtgtcctcaaatttctcggacccttc

SEQ ID NO: 101 - DNA, 60 bp, Zea mays
ctcggccaggatttcgagccaatggggtaacttcttattggacgaaatctccagcgggaa

SEQ ID NO: 102 - DNA, 60 bp, Zea mays
cggccaggatttcgagccaatggggtaagtccggcttcttattggacgaaatctccagcg

SEQ ID NO: 103 - DNA, 60 bp, Zea mays
tcggccaggatttcgagccaatggggtaagaaggagcgccggtttacatttaccgcacgt

SEQ ID NO: 104 - DNA, 60 bp, Zea mays
tcggccaggatttcgagccaatggggtaaggtagtaagaaggagcgccggtttacattta

SEQ ID NO: 105 - DNA, 60 bp, Zea mays
ggactacttcgtcggcaccctcggccaggagccggtttacatttaccgcacgtcggcgtg